SEQUENCE LISTING (88 sequences)

SEQ ID NO: 1 - DNA, 8 bp, Artificial Sequence
  cAMPRE
tgacgtca

SEQ ID NO: 2 - DNA, 7 bp, Artificial Sequence
  AP1 site consensus sequence
tgastca

SEQ ID NO: 3 - DNA, 7 bp, Artificial Sequence
  AP1(1)
tgagtca

SEQ ID NO: 4 - DNA, 8 bp, Artificial Sequence
  AP1(2)
tgactcag

SEQ ID NO: 5 - DNA, 5 bp, Artificial Sequence
  core consensus sequence for HBS
  misc_feature (1)..(1): n is a, c, g, or t
ncgtg

SEQ ID NO: 6 - DNA, 5 bp, Artificial Sequence
  HBS consensus sequence
rcgtg

SEQ ID NO: 7 - DNA, 9 bp, Artificial Sequence
  HRE1
ctgcacgta

SEQ ID NO: 8 - DNA, 6 bp, Artificial Sequence
  TFBS for HIF
acgtgc

SEQ ID NO: 9 - DNA, 30 bp, Artificial Sequence
  TFBS for HIF
accttgagtacgtgcgtctctgcacgtatg

SEQ ID NO: 10 - DNA, 6 bp, Artificial Sequence
  TFBS for ATF6 consensus sequence
tgacgt

SEQ ID NO: 11 - DNA, 7 bp, Artificial Sequence
  TFBS for ATF6 consensus sequence
tgacgtg

SEQ ID NO: 12 - DNA, 9 bp, Artificial Sequence
  TFBS sequence
tgacgtgct

SEQ ID NO: 13 - DNA, 61 bp, Artificial Sequence
  CRE comprising 5x cAMPRE and 3x AP1 TFBS
  misc_feature (8)..(9): optional spacer from 2 to 100 nucleotides
  misc_feature (16)..(17): optional spacer from 2 to 100 nucleotides
  misc_feature (24)..(25): optional spacer from 2 to 100 nucleotides
  misc_feature (32)..(33): optional spacer from 2 to 100 nucleotides
  misc_feature (40)..(41): optional spacer from 2 to 100 nucleotides
  misc_feature (47)..(48): optional spacer from 2 to 100 nucleotides
  misc_feature (54)..(55): optional spacer from 2 to 100 nucleotides
tgacgtcatgacgtcatgacgtcatgacgtcatgacgtcatgastcatgastcatgastca

SEQ ID NO: 14 - DNA, 68 bp, Artificial Sequence
  CRE comprising 5x cAMPRE and 4x AP1 TFBS
  misc_feature (8)..(9): optional spacer from 2 to 100 nucleotides
  misc_feature (16)..(17): optional spacer from 2 to 100 nucleotides
  misc_feature (24)..(25): optional spacer from 2 to 100 nucleotides
  misc_feature (32)..(33): optional spacer from 2 to 100 nucleotides
  misc_feature (40)..(41): optional spacer from 2 to 100 nucleotides
  misc_feature (47)..(48): optional spacer from 2 to 100 nucleotides
  misc_feature (54)..(55): optional spacer from 2 to 100 nucleotides
  misc_feature (61)..(62): optional spacer from 2 to 100 nucleotides
tgacgtcatgacgtcatgacgtcatgacgtcatgacgtcatgastcatgastcatgastcatgastca

SEQ ID NO: 15 - DNA, 56 bp, Artificial Sequence
  CRE comprising 8x AP1 TFBS
  misc_feature (7)..(8): optional spacer from 2 to 100 nucleotides
  misc_feature (14)..(15): optional spacer from 2 to 100 nucleotides
  misc_feature (21)..(22): optional spacer from 2 to 100 nucleotides
  misc_feature (28)..(29): optional spacer from 2 to 100 nucleotides
  misc_feature (35)..(36): optional spacer from 2 to 100 nucleotides
  misc_feature (42)..(43): optional spacer from 2 to 100 nucleotides
  misc_feature (49)..(50): optional spacer from 2 to 100 nucleotides
tgastcatgastcatgastcatgastcatgastcatgastcatgastcatgastca

SEQ ID NO: 16 - DNA, 61 bp, Artificial Sequence
  CRE 3x ATF6, 4x AP1 and 3x HIF TFBS
  misc_feature (6)..(7): optional spacer from 2 to 100 nucleotides
  misc_feature (12)..(13): optional spacer from 2 to 100 nucleotides
  misc_feature (18)..(19): optional spacer from 2 to 100 nucleotides
  misc_feature (25)..(26): optional spacer from 2 to 100 nucleotides
  misc_feature (32)..(33): optional spacer from 2 to 100 nucleotides
  misc_feature (39)..(40): optional spacer from 2 to 100 nucleotides
  misc_feature (46)..(47): optional spacer from 2 to 100 nucleotides
  misc_feature (51)..(52): optional spacer from 2 to 100 nucleotides
  misc_feature (56)..(57): optional spacer from 2 to 100 nucleotides
tgacgttgacgttgacgttgastcatgastcatgastcatgastcarcgtgrcgtgrcgtg

SEQ ID NO: 17 - DNA, 241 bp, Artificial Sequence
  Synp-FORCYB1
ccattgacgtcacgattaccattgacgtcacgattaccattgacgtcacgattaccattgacgtcacgattaccattgacgtcagcgattaagatgactcagcgattaagatgactcagcgattaagatgactcagcgattaagatgactcagcgattaatccatatgctctagagggtatataatgggggccactagtctactaccagaaagcttggtaccgagctcggatccagccacc

SEQ ID NO: 18 - DNA, 64 bp, Artificial Sequence
  CRE from Synp-FORCSV-10, comprising 5x cAMPRE and 3x AP1(2) TFBS
  misc_feature (8)..(9): optional spacer from 2 to 100 nucleotides
  misc_feature (16)..(17): optional spacer from 2 to 100 nucleotides
  misc_feature (24)..(25): optional spacer from 2 to 100 nucleotides
  misc_feature (32)..(33): optional spacer from 2 to 100 nucleotides
  misc_feature (40)..(41): optional spacer from 2 to 100 nucleotides
  misc_feature (48)..(49): optional spacer from 2 to 100 nucleotides
  misc_feature (56)..(57): optional spacer from 2 to 100 nucleotides
tgacgtcatgacgtcatgacgtcatgacgtcatgacgtcatgactcagtgactcagtgactcag

SEQ ID NO: 19 - DNA, 72 bp, Artificial Sequence
  CRE from Synp-FORCMV-09 comprising 5x cAMPRE and 4x AP1(2) TFBS
  misc_feature (8)..(9): optional spacer from 2 to 100 nucleotides
  misc_feature (16)..(17): optional spacer from 2 to 100 nucleotides
  misc_feature (24)..(25): optional spacer from 2 to 100 nucleotides
  misc_feature (32)..(33): optional spacer from 2 to 100 nucleotides
  misc_feature (40)..(41): optional spacer from 2 to 100 nucleotides
  misc_feature (48)..(49): optional spacer from 2 to 100 nucleotides
  misc_feature (56)..(57): optional spacer from 2 to 100 nucleotides
  misc_feature (64)..(65): optional spacer from 2 to 100 nucleotides
tgacgtcatgacgtcatgacgtcatgacgtcatgacgtcatgactcagtgactcagtgactcagtgactcag

SEQ ID NO: 20 - DNA, 56 bp, Artificial Sequence
  CRE from Synp-FMP-02, Synp-FLP-01 and Synp-RTV-017 comprising 8x AP1(1) TFBS
  misc_feature (7)..(8): optional spacer from 2 to 100 nucleotides
  misc_feature (14)..(15): optional spacer from 2 to 100 nucleotides
  misc_feature (21)..(22): optional spacer from 2 to 100 nucleotides
  misc_feature (28)..(29): optional spacer from 2 to 100 nucleotides
  misc_feature (35)..(36): optional spacer from 2 to 100 nucleotides
  misc_feature (42)..(43): optional spacer from 2 to 100 nucleotides
  misc_feature (49)..(50): optional spacer from 2 to 100 nucleotides
tgagtcatgagtcatgagtcatgagtcatgagtcatgagtcatgagtcatgagtca

SEQ ID NO: 21 - DNA, 82 bp, Artificial Sequence
  CRE from Synp-RTV-019 comprising 3x ATF6, 4x AP1(1) and 3x HRE1 TFBS
  misc_feature (9)..(10): optional spacer from 2 to 100 nucleotides
  misc_feature (18)..(19): optional spacer from 2 to 100 nucleotides
  misc_feature (27)..(28): optional spacer from 2 to 100 nucleotides
  misc_feature (34)..(35): optional spacer from 2 to 100 nucleotides
  misc_feature (41)..(42): optional spacer from 2 to 100 nucleotides
  misc_feature (48)..(49): optional spacer from 2 to 100 nucleotides
  misc_feature (55)..(56): optional spacer from 2 to 100 nucleotides
  misc_feature (64)..(65): optional spacer from 2 to 100 nucleotides
  misc_feature (73)..(74): optional spacer from 2 to 100 nucleotides
tgacgtgcttgacgtgcttgacgtgcttgagtcatgagtcatgagtcatgagtcactgcacgtactgcacgtactgcacgta

SEQ ID NO: 22 - DNA, 68 bp, Artificial Sequence
  CRE from Synp-FORCYB1 comprising 5x cAMPRE and 4x AP1(1) TFBS
  misc_feature (8)..(9): optional spacer from 2 to 100 nucleotides
  misc_feature (16)..(17): optional spacer from 2 to 100 nucleotides
  misc_feature (24)..(25): optional spacer from 2 to 100 nucleotides
  misc_feature (32)..(33): optional spacer from 2 to 100 nucleotides
  misc_feature (40)..(41): optional spacer from 2 to 100 nucleotides
  misc_feature (47)..(48): optional spacer from 2 to 100 nucleotides
  misc_feature (54)..(55): optional spacer from 2 to 100 nucleotides
  misc_feature (61)..(62): optional spacer from 2 to 100 nucleotides
tgacgtcatgacgtcatgacgtcatgacgtcatgacgtcatgactcatgactcatgactcatgactca

SEQ ID NO: 23 - DNA, 132 bp, Artificial Sequence
  CRE from Synp-FORCSV-10, comprising 5x cAMPRE and 3x AP1(2) TFBS
tgacgtcacgattaccattgacgtcacgattaccattgacgtcacgattaccattgacgtcacgattaccattgacgtcagcgattaagatgactcagcgattaagatgactcagcgattaagatgactcag

SEQ ID NO: 24 - DNA, 149 bp, Artificial Sequence
  CRE from Synp-FORCMV-09 comprising 5x cAMPRE and 4x AP1(2) TFBS
tgacgtcacgattaccattgacgtcacgattaccattgacgtcacgattaccattgacgtcacgattaccattgacgtcagcgattaagatgactcagcgattaagatgactcagcgattaagatgactcagcgattaagatgactcag

SEQ ID NO: 25 - DNA, 196 bp, Artificial Sequence
  CRE from Synp-FMP-02, Synp-FLP-01 and Synp-RTV-017 comprising 8x AP1(1) TFBS
tgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtca

SEQ ID NO: 26 - DNA, 262 bp, Artificial Sequence
  CRE from Synp-RTV-019 comprising 3x ATF6, 4x AP1(1) and 3x HRE1 TFBS
tgacgtgctgatgatgcgtagctagtagttgacgtgctgatgatgcgtagctagtagttgacgtgctgatgatgcgtagctagtagttgagtcagatgatgcgtagctagtagttgagtcagatgatgcgtagctagtagttgagtcagatgatgcgtagctagtagttgagtcagatgatgcgtagctagtagtctgcacgtagatgatgcgtagctagtagtctgcacgtagatgatgcgtagctagtagtctgcacgta

SEQ ID NO: 27 - DNA, 148 bp, Artificial Sequence
  CRE from Synp-FORCYB1 comprising 5x cAMPRE and 4x AP1(1) TFBS
tgacgtcacgattaccattgacgtcacgattaccattgacgtcacgattaccattgacgtcacgattaccattgacgtcagcgattaagatgactcagcgattaagatgactcagcgattaagatgactcagcgattaagatgactca

SEQ ID NO: 28 - DNA, 91 bp, Artificial Sequence
  CMV-MP
aggtctatataagcagagctcgtttagtgaaccgtcagatcgcctagatacgccatccacgctgttttgacctccatagaagatcgccacc

SEQ ID NO: 29 - DNA, 89 bp, Artificial Sequence
  YB-TATA
gcgattaatccatatgctctagagggtatataatgggggccactagtctactaccagaaagcttggtaccgagctcggatccagccacc

SEQ ID NO: 30 - DNA, 63 bp, Artificial Sequence
  MinTK MP
ttcgcatattaaggtgacgcgtgtggcctcgaacaccgagcgaccctgcagcgacccgcttaa

SEQ ID NO: 31 - DNA, 197 bp, Artificial Sequence
  SV40-MP
tgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 32 - DNA, 76 bp, Artificial Sequence
  G6PC-MP
gggcatataaaacaggggcaaggcacagactcatagcagagcaatcaccaccaagcctggaataactgcagccacc

SEQ ID NO: 33 - DNA, 258 bp, Artificial Sequence
  CRE comprising 5x cAMPRE and 3x AP1 TFBS and SV40-MP
  misc_feature (8)..(9): optional spacer from 2 to 100 nucleotides
  misc_feature (16)..(17): optional spacer from 2 to 100 nucleotides
  misc_feature (24)..(25): optional spacer from 2 to 100 nucleotides
  misc_feature (32)..(33): optional spacer from 2 to 100 nucleotides
  misc_feature (40)..(41): optional spacer from 2 to 100 nucleotides
  misc_feature (47)..(48): optional spacer from 2 to 100 nucleotides
  misc_feature (54)..(55): optional spacer from 2 to 100 nucleotides
  misc_feature (61)..(62): optional spacer from 2 to 100 nucleotides
tgacgtcatgacgtcatgacgtcatgacgtcatgacgtcatgastcatgastcatgastcatgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 34 - DNA, 159 bp, Artificial Sequence
  CRE comprising 5x cAMPRE and 4x AP1 TFBS and CMV-MP
  misc_feature (8)..(9): optional spacer from 2 to 100 nucleotides
  misc_feature (16)..(17): optional spacer from 2 to 100 nucleotides
  misc_feature (24)..(25): optional spacer from 2 to 100 nucleotides
  misc_feature (32)..(33): optional spacer from 2 to 100 nucleotides
  misc_feature (40)..(41): optional spacer from 2 to 100 nucleotides
  misc_feature (47)..(48): optional spacer from 2 to 100 nucleotides
  misc_feature (54)..(55): optional spacer from 2 to 100 nucleotides
  misc_feature (61)..(62): optional spacer from 2 to 100 nucleotides
  misc_feature (68)..(69): optional spacer from 2 to 100 nucleotides
tgacgtcatgacgtcatgacgtcatgacgtcatgacgtcatgastcatgastcatgastcatgastcaaggtctatataagcagagctcgtttagtgaaccgtcagatcgcctagatacgccatccacgctgttttgacctccatagaagatcgccacc

SEQ ID NO: 35 - DNA, 119 bp, Artificial Sequence
  CRE comprising 8x AP1 TFBS and Min-TK
  misc_feature (7)..(8): optional spacer from 2 to 100 nucleotides
  misc_feature (14)..(15): optional spacer from 2 to 100 nucleotides
  misc_feature (21)..(22): optional spacer from 2 to 100 nucleotides
  misc_feature (28)..(29): optional spacer from 2 to 100 nucleotides
  misc_feature (35)..(36): optional spacer from 2 to 100 nucleotides
  misc_feature (42)..(43): optional spacer from 2 to 100 nucleotides
  misc_feature (49)..(50): optional spacer from 2 to 100 nucleotides
  misc_feature (56)..(57): optional spacer from 2 to 100 nucleotides
tgastcatgastcatgastcatgastcatgastcatgastcatgastcatgastcattcgcatattaaggtgacgcgtgtggcctcgaacaccgagcgaccctgcagcgacccgcttaa

SEQ ID NO: 36 - DNA, 152 bp, Artificial Sequence
  CRE 3x ATF6, 4x AP1 and 3x HIF TFBS and CMV-MP
  misc_feature (6)..(7): optional spacer from 2 to 100 nucleotides
  misc_feature (12)..(13): optional spacer from 2 to 100 nucleotides
  misc_feature (18)..(19): optional spacer from 2 to 100 nucleotides
  misc_feature (25)..(26): optional spacer from 2 to 100 nucleotides
  misc_feature (32)..(33): optional spacer from 2 to 100 nucleotides
  misc_feature (39)..(40): optional spacer from 2 to 100 nucleotides
  misc_feature (46)..(47): optional spacer from 2 to 100 nucleotides
  misc_feature (51)..(52): optional spacer from 2 to 100 nucleotides
  misc_feature (56)..(57): optional spacer from 2 to 100 nucleotides
  misc_feature (61)..(62): optional spacer from 2 to 100 nucleotides
tgacgttgacgttgacgttgastcatgastcatgastcatgastcarcgtgrcgtgrcgtgaggtctatataagcagagctcgtttagtgaaccgtcagatcgcctagatacgccatccacgctgttttgacctccatagaagatcgccacc

SEQ ID NO: 37 - DNA, 157 bp, Artificial Sequence
  CRE comprising 5x cAMPRE and 4x AP1 TFBS and YB-TATA
  misc_feature (8)..(9): optional spacer from 2 to 100 nucleotides
  misc_feature (16)..(17): optional spacer from 2 to 100 nucleotides
  misc_feature (24)..(25): optional spacer from 2 to 100 nucleotides
  misc_feature (32)..(33): optional spacer from 2 to 100 nucleotides
  misc_feature (40)..(41): optional spacer from 2 to 100 nucleotides
  misc_feature (47)..(48): optional spacer from 2 to 100 nucleotides
  misc_feature (54)..(55): optional spacer from 2 to 100 nucleotides
  misc_feature (61)..(62): optional spacer from 2 to 100 nucleotides
  misc_feature (68)..(69): optional spacer from 2 to 100 nucleotides
tgacgtcatgacgtcatgacgtcatgacgtcatgacgtcatgastcatgastcatgastcatgastcagcgattaatccatatgctctagagggtatataatgggggccactagtctactaccagaaagcttggtaccgagctcggatccagccacc

SEQ ID NO: 38 - DNA, 61 bp, Artificial Sequence; identical to SEQ ID NO: 16
  synthetic forskolin-inducible promoter
  misc_feature (6)..(7): optional spacer of 20 nucleotides
  misc_feature (12)..(13): optional spacer of 20 nucleotides
  misc_feature (18)..(19): optional spacer of 20 nucleotides
  misc_feature (25)..(26): optional spacer of 20 nucleotides
  misc_feature (32)..(33): optional spacer of 20 nucleotides
  misc_feature (39)..(40): optional spacer of 20 nucleotides
  misc_feature (46)..(47): optional spacer of 20 nucleotides
  misc_feature (51)..(52): optional spacer of 20 nucleotides
  misc_feature (56)..(57): optional spacer of 20 nucleotides
  misc_feature (61)..(62): optional spacer of 61nucleotides and minimal promoter CMV-MP
  misc_feature (61)..(62): optional spacer of 61 nucleotides and minimal promoter CMV-MP

SEQ ID NO: 39 - DNA, 371 bp, Artificial Sequence
  synthetic forskolin-inducible promoter
tgacgtcacgattaccattgacgtcacgattaccattgacgtcacgattaccattgacgtcacgattaccattgacgtcagcgattaagatgactcagcgattaagatgactcagcgattaagatgactcagcgattaagatgactcactagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 40 - DNA, 256 bp, Artificial Sequence
  synthetic forskolin-inducible promoter
tgacgtcacgattaccattgacgtcacgattaccattgacgtcacgattaccattgacgtcacgattaccattgacgtcagcgattaagatgactcagcgattaagatgactcagcgattaagatgactcagcgattaagatgactcagcgattaatccatatgcaggtctatataagcagagctcgtttagtgaaccgtcagatcgcctagatacgccatccacgctgttttgacctccatagaagatcgccacc

SEQ ID NO: 41 - DNA, 318 bp, Artificial Sequence
  Synp-FMP-02
tgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagtagtcgtatgctgatgcgcagttagcgtagctgaggtaccgtcgacgatatcggatccttcgcatattaaggtgacgcgtgtggcctcgaacaccgagcgaccctgcagcgacccgcttaa

SEQ ID NO: 42 - DNA, 331 bp, Artificial Sequence
  Synp-FLP-01
tgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagtagtcgtatgctgatgcgcagttagcgtagctgaggtaccgtcgacgatatcggatccgggcatataaaacaggggcaaggcacagactcatagcagagcaatcaccaccaagcctggaataactgcagccacc

SEQ ID NO: 43 - DNA, 346 bp, Artificial Sequence
  Synp-RTV-017
tgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagtagtcgtatgctgatgcgcagttagcgtagctgaggtaccgtcgacgatatcggatccaggtctatataagcagagctcgtttagtgaaccgtcagatcgcctagatacgccatccacgctgttttgacctccatagaagatcgccacc

SEQ ID NO: 44 - DNA, 414 bp, Artificial Sequence
  Synp-RTV-019
tgacgtgctgatgatgcgtagctagtagttgacgtgctgatgatgcgtagctagtagttgacgtgctgatgatgcgtagctagtagttgagtcagatgatgcgtagctagtagttgagtcagatgatgcgtagctagtagttgagtcagatgatgcgtagctagtagttgagtcagatgatgcgtagctagtagtctgcacgtagatgatgcgtagctagtagtctgcacgtagatgatgcgtagctagtagtctgcacgtagatgatgcgtagctagtagtgcagttagcgtagctgaggtaccgtcgacgatatcggatccaggtctatataagcagagctcgtttagtgaaccgtcagatcgcctagatacgccatccacgctgttttgacctccatagaagatcgccacc

SEQ ID NO: 45 - DNA, 237 bp, Artificial Sequence
  synthetic forskolin-inducible promoter
tgacgtcacgattaccattgacgtcacgattaccattgacgtcacgattaccattgacgtcacgattaccattgacgtcagcgattaagatgactcagcgattaagatgactcagcgattaagatgactcagcgattaagatgactcagcgattaatccatatgctctagagggtatataatgggggccactagtctactaccagaaagcttggtaccgagctcggatccagccacc

SEQ ID NO: 46 - DNA, 410 bp, Artificial Sequence
  Synp-FORCSV-10
ccattgacgtcacgattaccattgacgtcacgattaccattgacgtcacgattaccattgacgtcacgattaccattgacgtcagcgattaagatgactcagcgattaagatgactcagcgattaagatgactcagcgattaagatgactcactagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccacc

SEQ ID NO: 47 - DNA, 260 bp, Artificial Sequence
  Synp-FORCMV-09
ccattgacgtcacgattaccattgacgtcacgattaccattgacgtcacgattaccattgacgtcacgattaccattgacgtcagcgattaagatgactcagcgattaagatgactcagcgattaagatgactcagcgattaagatgactcagcgattaatccatatgcaggtctatataagcagagctcgtttagtgaaccgtcagatcgcctagatacgccatccacgctgttttgacctccatagaagatcgccacc

SEQ ID NO: 48 - DNA, 4030 bp, Artificial Sequence
  PM-RQ vector
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaagtgtaatgaaagcttggtctctacgagtaatagacgcccagttgaattccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtaacaacaacaattgcattcattttatgtttcaggttcagggggaggtgtgggaggttttttaaagcaagtaaaacctctacaaatgtggtaaaatcgataaggatccgtctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 49 - DNA, 255 bp, Artificial Sequence
  SEAP coding sequence
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagataccc

SEQ ID NO: 50 - DNA, 3412 bp, Artificial Sequence
  pAAV vector
cgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatc

SEQ ID NO: 51 - DNA, 132 bp, Artificial Sequence
  CRE comprising 8x AP1 TFBS and G6PC MP
  misc_feature (7)..(8): optional spacer from 2 to 100 nucleotides
  misc_feature (14)..(15): optional spacer from 2 to 100 nucleotides
  misc_feature (21)..(22): optional spacer from 2 to 100 nucleotides
  misc_feature (28)..(29): optional spacer from 2 to 100 nucleotides
  misc_feature (35)..(36): optional spacer from 2 to 100 nucleotides
  misc_feature (42)..(43): optional spacer from 2 to 100 nucleotides
  misc_feature (49)..(50): optional spacer from 2 to 100 nucleotides
  misc_feature (56)..(57): optional spacer from 2 to 100 nucleotides
tgastcatgastcatgastcatgastcatgastcatgastcatgastcatgastcagggcatataaaacaggggcaaggcacagactcatagcagagcaatcaccaccaagcctggaataactgcagccacc

SEQ ID NO: 52 - DNA, 147 bp, Artificial Sequence
  CRE comprising 8x AP1 TFBS and CMV-MP
  misc_feature (7)..(8): optional spacer from 2 to 100 nucleotides
  misc_feature (14)..(15): optional spacer from 2 to 100 nucleotides
  misc_feature (21)..(22): optional spacer from 2 to 100 nucleotides
  misc_feature (28)..(29): optional spacer from 2 to 100 nucleotides
  misc_feature (35)..(36): optional spacer from 2 to 100 nucleotides
  misc_feature (42)..(43): optional spacer from 2 to 100 nucleotides
  misc_feature (49)..(50): optional spacer from 2 to 100 nucleotides
  misc_feature (56)..(57): optional spacer from 2 to 100 nucleotides
tgastcatgastcatgastcatgastcatgastcatgastcatgastcatgastcaaggtctatataagcagagctcgtttagtgaaccgtcagatcgcctagatacgccatccacgctgttttgacctccatagaagatcgccacc

SEQ ID NO: 53 - DNA, 4483 bp, Artificial Sequence
  pMA-RQ luciferase vector - RTV-016
taatacgactcactatagggcgaattggcggaaggccgtcaaggccgcatgcataataaaatatctttattttcattacatctgtgtgttggttttttgtgtgctgcacgtagatgatgcgtagctagtagtctgcacgtaagatgatgcgtagctagtagtctgcacgtagatgatgcgtagctagtagtctgcacgtagatgatgcgtagctagtagtctgcacgtagatgatgcgtagctagtagtctgcacgtagtagtcgtatgctgatgcgcagttagcgtagctgaggtaccgtcgacgatatcggatccaggtctatataagcagagctcgtttagtgaaccgtcagatcgcctagatacgccatccacgctgttttgacctccatagaagatcgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgaaagcttggtctctacgagtaatagacgcccagttgaattccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtctgggcctcatgggccttccgctcactgcccgctttccagtcgggaaacctgacaacaacaattgcattcattttatgtttcaggttcagggggaggtgtgggaggttttttaaagcaagtaaaacctctacaaatgtggtaaaatcgataaggatccgtctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 54 - DNA, 4605 bp, Artificial Sequence
  pMA-RQ luciferase vector - HYBT
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgcataataaaatatctttattttcattacatctgtgtgttggttttttgtgtggacggatcgggagatctttgtatttaattaagaccttgagtacgtgcgtctctgcacgtatggcgattaagaccttgagtacgtgcgtctctgcacgtatggcgattaagaccttgagtacgtgcgtctctgcacgtatggcgattaagaccttgagtacgtgcgtctctgcacgtatggcgattaatccatatgctctagagggtatataatgggggccactagtctactaccagaaagcttggtaccgagctcggatccagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgaaagcttggtctctacgagtaatagacgcccagttgaattccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 55 - DNA, 4624 bp, Artificial Sequence
  pMA-RQ luciferase vector - HV3C
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgcataataaaatatctttattttcattacatctgtgtgttggttttttgtgtggacggatcgggagatctttgtatttaattaagaccttgagtacgtgcgtctctgcacgtatggcgattaagaccttgagtacgtgcgtctctgcacgtatggcgattaagaccttgagtacgtgcgtctctgcacgtatggcgattaagaccttgagtacgtgcgtctctgcacgtatggcgattaatccatatgcaggtctatataagcagagctcgtttagtgaaccgtcagatcgcctagatacgccatccacgctgttttgacctccatagaagatcgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgaaagcttggtctctacgagtaatagacgcccagttgaattccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 56 - DNA, 4199 bp, Artificial Sequence
  pMA-RQ luciferase vector - Synp-HYP-001
aataaaatatctttattttcattacatctgtgtgttggttttttgtgtgctgcacgtactgcacgtactgcacgtactgcacgtatgggtaccgtcgacgatatcggatccaggtctatataagcagagctcgtttagtgaaccgtcagatcgcctagatacgccatccacgctgttttgacctccatagaagatcgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgaaagcttggtctctacgagtaatagacgcccagttgaattccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaa

SEQ ID NO: 57 - DNA, 25 bp, Artificial Sequence
  sYB-TATA
tctagagggtatataatgggggcca

SEQ ID NO: 58 - DNA, 5 bp, Artificial Sequence
  HBS reverse complement consensus sequence
cacgy

SEQ ID NO: 59 - DNA, 6 bp, Artificial Sequence
  HRE1 reverse complement sequence
gcacgt

SEQ ID NO: 60 - DNA, 9 bp, Artificial Sequence
  HRE2 reverse complement sequence
tacgtgcag

SEQ ID NO: 61 - DNA, 30 bp, Artificial Sequence
  HRE3 reverse complement sequence
catacgtgcagagacgcacgtactcaaggt

SEQ ID NO: 62 - DNA, 14 bp, Artificial Sequence
  functional variant of HRE3
  misc_feature (1)..(1): optional spacer from 8 to 10 nucleotides
  misc_feature (5)..(6): optional spacer from 4 to 6 nucleotides
  misc_feature (14)..(14): optional spacer from 1 to 3 nucleotides
acgtgctgcacgta

SEQ ID NO: 63 - DNA, 30 bp, Artificial Sequence
  functional variant of HRE3
  misc_feature (1)..(9): n is a, c, g, or t
  misc_feature (15)..(19): n is a, c, g, or t
  misc_feature (29)..(30): n is a, c, g, or t
nnnnnnnnnacgtgnnnnnctgcacgtann

SEQ ID NO: 64 - DNA, 12 bp, Artificial Sequence
  HRE
  repeat_unit (1)..(6): repeat unit, from 2 to 9 repeats
  misc_feature (6)..(7): optional spacer from 2 to 100 nucleotides
acgtgcacgtgc

SEQ ID NO: 65 - DNA, 6 bp, Artificial Sequence
  HRE repeat unit
  repeat_unit (1)..(6): repeat unit, from 2 to 9 repeats
  misc_feature (6)..(6): optional spacer from 2 to 100 nucleotides
acgtgc

SEQ ID NO: 66 - DNA, 30 bp, Artificial Sequence
  HRE
  misc_feature (6)..(7): optional spacer from 2 to 100 nucleotides
  misc_feature (12)..(13): optional spacer from 2 to 100 nucleotides
  misc_feature (18)..(19): optional spacer from 2 to 100 nucleotides
  misc_feature (24)..(25): optional spacer from 2 to 100 nucleotides
acgtgcacgtgcacgtgcacgtgcacgtgc

SEQ ID NO: 67 - DNA, 40 bp, Artificial Sequence
  spacer
gatgatgcgtagctagtagtgatgatgcgtagctagtagt

SEQ ID NO: 68 - DNA, 190 bp, Artificial Sequence
  HRE
acgtgcgatgatgcgtagctagtagtgatgatgcgtagctagtagtacgtgcgatgatgcgtagctagtagtgatgatgcgtagctagtagtacgtgcgatgatgcgtagctagtagtgatgatgcgtagctagtagtacgtgcgatgatgcgtagctagtagtgatgatgcgtagctagtagtacgtgc

SEQ ID NO: 69 - DNA, 18 bp, Artificial Sequence
  HRE
  repeat_unit (1)..(9): repeat unit, from 2 to 9 repeats
  misc_feature (9)..(10): optional spacer from 2 to 100 nucleotides
ctgcacgtactgcacgta

SEQ ID NO: 70 - DNA, 9 bp, Artificial Sequence
  HRE repeat unit
  repeat_unit (1)..(9): repeat unit, from 2 to 9 repeats
  misc_feature (9)..(9): optional spacer from 2 to 100 nucleotides
ctgcacgta

SEQ ID NO: 71 - DNA, 54 bp, Artificial Sequence
  HRE
  misc_feature (9)..(10): optional spacer from 2 to 100 nucleotides
  misc_feature (18)..(19): optional spacer from 2 to 100 nucleotides
  misc_feature (27)..(28): optional spacer from 2 to 100 nucleotides
  misc_feature (36)..(37): optional spacer from 2 to 100 nucleotides
  misc_feature (45)..(46): optional spacer from 2 to 100 nucleotides
ctgcacgtactgcacgtactgcacgtactgcacgtactgcacgtactgcacgta

SEQ ID NO: 72 - DNA, 20 bp, Artificial Sequence
  spacer
gatgatgcgtagctagtagt

SEQ ID NO: 73 - DNA, 154 bp, Artificial Sequence
  HRE
ctgcacgtagatgatgcgtagctagtagtctgcacgtagatgatgcgtagctagtagtctgcacgtagatgatgcgtagctagtagtctgcacgtagatgatgcgtagctagtagtctgcacgtagatgatgcgtagctagtagtctgcacgta

SEQ ID NO: 74 - DNA, 36 bp, Artificial Sequence
  HRE
ctgcacgtactgcacgtactgcacgtactgcacgta

SEQ ID NO: 75 - DNA, 60 bp, Artificial Sequence
  HRE
  repeat_unit (1)..(30): repeat unit, from 2 to 5 repeats
  misc_feature (30)..(31): optional spacer from 2 to 100 nucleotides
accttgagtacgtgcgtctctgcacgtatgaccttgagtacgtgcgtctctgcacgtatg

SEQ ID NO: 76 - DNA, 30 bp, Artificial Sequence; identical to SEQ ID NO: 9
  repeat unit
  repeat_unit (1)..(30): repeat unit, from 2 to 5 repeats
  misc_feature (30)..(30): optional spacer from 2 to 100 nucleotides

SEQ ID NO: 77 - DNA, 150 bp, Artificial Sequence
  HRE
  misc_feature (30)..(31): optional spacer from 2 to 100 nucleotides
  misc_feature (60)..(61): optional spacer from 2 to 100 nucleotides
  misc_feature (90)..(91): optional spacer from 2 to 100 nucleotides
  misc_feature (120)..(121): optional spacer from 2 to 100 nucleotides
  misc_feature (150)..(150): optional spacer from 2 to 100 nucleotides
accttgagtacgtgcgtctctgcacgtatgaccttgagtacgtgcgtctctgcacgtatgaccttgagtacgtgcgtctctgcacgtatgaccttgagtacgtgcgtctctgcacgtatgaccttgagtacgtgcgtctctgcacgtatg

SEQ ID NO: 78 - DNA, 9 bp, Artificial Sequence
  spacer
gcgattaag

SEQ ID NO: 79 - DNA, 147 bp, Artificial Sequence
  HRE
accttgagtacgtgcgtctctgcacgtatggcgattaagaccttgagtacgtgcgtctctgcacgtatggcgattaagaccttgagtacgtgcgtctctgcacgtatggcgattaagaccttgagtacgtgcgtctctgcacgtatg

SEQ ID NO: 80 - DNA, 131 bp, Artificial Sequence
  MP1
ttggtaccatccgggccggccgcttaagcgacgcctataaaaaataggttgcatgctaggcctagcgctgccagtccatcttcgctagcctgtgctgcgtcagtccagcgctgcgctgcgtaacggccgcc

SEQ ID NO: 81 - DNA, 321 bp, Artificial Sequence
  Synp-RTV-015
acgtgcgatgatgcgtagctagtagtgatgatgcgtagctagtagtacgtgcgatgatgcgtagctagtagtgatgatgcgtagctagtagtacgtgcgatgatgcgtagctagtagtgatgatgcgtagctagtagtacgtgcgatgatgcgtagctagtagtgatgatgcgtagctagtagtacgtgcttggtaccatccgggccggccgcttaagcgacgcctataaaaaataggttgcatgctaggcctagcgctgccagtccatcttcgctagcctgtgctgcgtcagtccagcgctgcgctgcgtaacggccgcc

SEQ ID NO: 82 - DNA, 304 bp, Artificial Sequence
  Synp-RTV-016
ctgcacgtagatgatgcgtagctagtagtctgcacgtagatgatgcgtagctagtagtctgcacgtagatgatgcgtagctagtagtctgcacgtagatgatgcgtagctagtagtctgcacgtagatgatgcgtagctagtagtctgcacgtagtagtcgtatgctgatgcgcagttagcgtagctgaggtaccgtcgacgatatcggatccaggtctatataagcagagctcgtttagtgaaccgtcagatcgcctagatacgccatccacgctgttttgacctccatagaagatcgccacc

SEQ ID NO: 83 - DNA, 153 bp, Artificial Sequence
  Synp-HYP-001
ctgcacgtactgcacgtactgcacgtactgcacgtatgggtaccgtcgacgatatcggatccaggtctatataagcagagctcgtttagtgaaccgtcagatcgcctagatacgccatccacgctgttttgacctccatagaagatcgccacc

SEQ ID NO: 84 - DNA, 189 bp, Artificial Sequence
  part of Synp-HYBT
accttgagtacgtgcgtctctgcacgtatggcgattaagaccttgagtacgtgcgtctctgcacgtatggcgattaagaccttgagtacgtgcgtctctgcacgtatggcgattaagaccttgagtacgtgcgtctctgcacgtatggcgattaatccatatgctctagagggtatataatgggggcca

SEQ ID NO: 85 - DNA, 255 bp, Artificial Sequence
  part of Synp-HV3C
accttgagtacgtgcgtctctgcacgtatggcgattaagaccttgagtacgtgcgtctctgcacgtatggcgattaagaccttgagtacgtgcgtctctgcacgtatggcgattaagaccttgagtacgtgcgtctctgcacgtatggcgattaatccatatgcaggtctatataagcagagctcgtttagtgaaccgtcagatcgcctagatacgccatccacgctgttttgacctccatagaagatcgccacc

SEQ ID NO: 86 - DNA, 256 bp, Artificial Sequence
  Synp-HYBT
gatctttgtatttaattaagaccttgagtacgtgcgtctctgcacgtatggcgattaagaccttgagtacgtgcgtctctgcacgtatggcgattaagaccttgagtacgtgcgtctctgcacgtatggcgattaagaccttgagtacgtgcgtctctgcacgtatggcgattaatccatatgctctagagggtatataatgggggccactagtctactaccagaaagcttggtaccgagctcggatccagccacc

SEQ ID NO: 87 - DNA, 275 bp, Artificial Sequence
  Synp-HV3C
gatctttgtatttaattaagaccttgagtacgtgcgtctctgcacgtatggcgattaagaccttgagtacgtgcgtctctgcacgtatggcgattaagaccttgagtacgtgcgtctctgcacgtatggcgattaagaccttgagtacgtgcgtctctgcacgtatggcgattaatccatatgcaggtctatataagcagagctcgtttagtgaaccgtcagatcgcctagatacgccatccacgctgttttgacctccatagaagatcgccacc

SEQ ID NO: 88 - DNA, 5465 bp, Artificial Sequence
  pMA-RQ luciferase vector - RTV-015
acgtgcgatgagctccccgggttaattaacatatgactagtgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggcggccgcgacctgcaggcgcagaactggtaggtatggaagatccctcgagatccattgtgctggcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgccagcacaatggatctcgaggtcgagggatctctagaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagccaagctagatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttggggccaccgctcagagcaccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaattagactttgagtgatcttgagcctttcctagttcatcccaccccgccccagagagatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctctagcttcgtgtcaaggacggtgagg

(19) United States
(12) Patent Application Publication
(10) Pub. No.: US 2023/0167458 A1
(43) Pub. Date: Jun. 1, 2023

(54) FORSKOLIN-INDUCIBLE PROMOTERS AND HYPOXIA-INDUCIBLE PROMOTERS

(71) Applicant: ASKLEPIOS BIOPHARMACEUTICAL, INC., Research Triangle Park, NC (US)

(72) Inventors: Graham WHYTESIDE, Edinburgh, Midlothian (GB); Victoria Fiona Torrance, Edinburgh, Midlothian (GB); Michael L. Roberts, Edinburgh, Midlothian (GB)

(73) Assignee: ASKLEPIOS BIOPHARMACEUTICAL, INC., Research Triangle Park, NC (US)

(21) Appl. No.: 17/918,277

(22) PCT Filed: Apr. 9, 2021
(86) PCT No.: PCT/GB2021/050871

§ 371 Date: Oct. 11, 2022

(30) Foreign Application Priority Data
Apr. 15, 2020 (GB) 2005473.0
Apr. 15, 2020 (GB) 2005475.5

(51) Int. Cl. C12N 15/85; C12P 21/00

(52) U.S. Cl. CPC C12N 15/85; C12P 21/00; C12N 2830/002

(57) ABSTRACT

The present invention relates to forskolin-inducible and hypoxia-inducible cis-regulatory elements, promoters and vectors, and methods of their use.

### CROSS REFERENCE TO RELATED APPLICATIONS

This application is a 35 U.S.C. § 371 National Phase Entry Application of International Application No. PCT/GB2021/050871 filed Apr. 9, 2021, which claims benefit under 35 U.S.C. § 119(b) of GB Application Nos. 2005473.0 filed Apr. 15, 2020 and 2005475.5 filed Apr. 15, 2020, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Apr. 16, 2021, is named pctgb2021050871-seql and is 83 KB in size.

### FIELD OF THE INVENTION

The present invention relates to forskolin-inducible cis-regulatory elements, promoters and vectors, and methods of their use. The present invention also related to hypoxia-inducible promoters and vectors, especially bioprocessing vectors, and methods of their use.

### BACKGROUND OF THE INVENTION

The following discussion is provided to aid the reader in understanding the disclosure and does not constitute any admission as to the contents or relevance of the prior art.

Therapeutic proteins/polypeptides or nucleic acids are increasingly used in the pharmaceutical industry. Therapeutic proteins tend to be produced in large quantity by genetically modified organisms in tightly regulated processes. The genetic modification is performed in order to allow the cells to express the recombinant expression product. Other forms of biological proteins (e.g. enzymes, antibodies and other useful proteins) are produced in similar processes.

Genetical modification generally consists of modifying the cells to include an expression cassette, usually in the form of a vector, which comprises a coding sequence encoding the expression product operably liked to a promoter. The promoter may be constitutively active or inducible.

Inducible promoters allow production of the expression product to be induced at a desired point, which is useful in many ways. An inducible promoter can be used to produce expression products which are toxic to cells or which would inhibit the growth of the cells, as it allows the cells to be grown to a specific density or number before inducing production of the expression product and harvesting. Inducible promoters can also be used to express co-factors which enhance the yield, potency or the stability of the expression product. Due to their usefulness, there is a demand for inducible promoters, preferably with minimal leakiness.

Inducible promoters known in the art include sugar-inducible promoters such as rhamnose promoter (WO2006/061174A2) and carbon source depletion inducible promoters such as pG1 (WO2013/050551A1). These promoters require the inclusion of a substance (such as rhamnose) or the withdrawal of a substance (such as carbon source) which has been present in the culture until this point.

These approaches have potential drawbacks. The substance to be added or withdrawn is potentially required in large quantity for a large-scale culture and this can be costly. Additionally, the presence of the substance might be undesirable in the final pharmaceutical product, for example if it is not safe for human consumption, and therefore there might be a need to ensure that the substance is completely removed. These approaches might also be time consuming as they require uniform mixing of a new substance in a large culture or depletion below a certain threshold of a substance which is already present in the culture. This substance would then have to be taken up in the cell, so it can influence gene expression, or depleted from the cell, so that repression of gene expression can be lifted. It is desirable to provide inducible promoters which overcome some or all of these drawbacks.

Similarly, in gene therapy, it is desirable to provide regulatory nucleic acid sequences that are capable of driving expression of a gene to produce a protein or nucleic acid expression product within the body. There is also a desire to provide inducible systems of gene expression, such that gene expression can be induced as required. Inducibility means that expression of a therapeutic gene expression product can be induced when required. Furthermore, if induction is dose dependent, then expression levels of therapeutic gene expression product can be modulated by adjusting the amount of inducer administered. It is desirable to provide inducible promoters which have some or all of these characteristics.

Thus, there is a need for inducible regulatory sequences to control gene expression in many contexts, not least in therapeutic gene expression in gene therapy and/or production of therapeutic expression products in bioprocessing.

Inducible promoters may be inducible by activators of adenylate cyclase by using the cAMPRE and/or AP1 TFBS. The ATP derivative cyclic adenosine monophosphate (also known as cAMP, cyclic AMP, or 3′,5′-cyclic adenosine monophosphate) is a second messenger important in many biological processes. Its main function is in intracellular signal transduction in many different organisms. The cAMP-dependent pathway has been well-studied, and it is reviewed in (Yan, et al., 2016).

Cyclic AMP is produced by activation of the adenylyl cyclase (also commonly known as adenyl cyclase and adenylate cyclase, abbreviated as AC). Activation of adenylyl cyclase drives a cascade that, via protein kinase A, leads to activation of the transcription factor CREB which binds specific TFBS, called cAMPRE, having the sequence TGACGTCA (SEQ ID NO: 1), to modulate gene expression.

Another indirect effect of activation of adenylyl cyclase is the subsequent activation of the transcription factor AP1. AP1 is a dimer composed of variations of Fos and Jun proteins of which there are many forms. These proteins have a complex regulation pathway involving many protein kinases, but elevated cAMP levels are believed to stabilise the protein c-Fos and upregulate its transcription, leading to activation of AP1. See, for example, (Hess, et al., 2004) and (Sharma & Richards, 2000). AP1 binds to specific TFBS, called AP1 sites and having the consensus sequence TGA[GC]TCA (SEQ ID NO: 2), to modulate gene expression.

The present invention presents novel synthetic CREs inducible by activators of adenylate cyclase by using the cAMPRE and/or AP1 TFBS.

Inducible promoters may be inducible by hypoxia. Cellular response to hypoxic conditions is conserved across all eukaryotes. The response to low oxygen stress is mediated by transcription factors known generally as hypoxia-inducible factors (HIFs) including HIF1 and HIF2. These factors are sensitive to reduced oxygen concentration within the cell. Oxygen sensitivity is achieved by degradation of one of the two subunits of HIF1, HIF1a, in normoxia and its stabilisation during hypoxia. In hypoxia, stabilisation of HIF1a results in dimerization of HIF1α and HIF1β which allows the HIF1 complex to upregulate the transcription of genes to mitigate this stress.

In order to modulate gene expression, HIF1 binds to hypoxia-responsive elements (HRE) which have HIF binding sites (HBS). HIF binding sites tend to have a consensus sequence, NCGTG (SEQ ID NO: 5) (Schödel, et al., 2011). Hypoxia-responsive elements can be used to create synthetic hypoxia-responsive promoters which drive expression of a product of interest in hypoxic condition but not in normoxia. Hypoxia-inducible promoters have been explored in gene therapy, particularly in cancer (Javan & Shanbazi, 2017).

The present invention provides a synthetic hypoxia-inducible bioprocessing promoter which overcome the drawbacks associated with the inducible promoters currently used in bioprocessing applications.

The requirements for an inducer in gene therapy and bioprocessing are somewhat different. In gene therapy, it is of paramount importance for the inducer to be safe and non-toxic for human and to be able to penetrate variety of tissues. In bioprocessing, the inducer must be suitable for distribution in a cell culture and, preferably, easy to wash out or otherwise remove if its presence is undesirable in the final product. It is an object of the present invention to provide for synthetic promoters inducible by AC activation or hypoxia, which can be activated both by inducer suitable for gene therapy and inducer suitable for bioprocessing.

### SUMMARY OF THE INVENTION

According to a first aspect of the invention, there is provided a synthetic forskolin-inducible cis-regulatory element (CRE) that is capable of being bound by CREB and/or AP1.

While the CRE is referred to as forskolin-inducible, it may also be induced by other agents, as discussed in more detail below. The mechanism of induction by forskolin is via the activation of adenylyl cyclase and the resultant increase of intracellular cAMP. Accordingly, the CRE is also inducible by other activators of adenylyl cyclase or factors that increase intracellular cAMP.

Preferably the CRE comprises at least 2, more preferably at least 3, transcription factor binding sites (TFBS) for CREB and/or AP1 (as used herein, the term “TFBS for X” means a TFBS which is capable of being bound by transcription factor X).

Preferably the CRE comprises at least 4 TFBS for CREB and/or AP1. Suitably the CRE comprises 3, 4, 5, 6, 7, 8, 9, 10, 11, 12, 13, 14, or 15 TFBS for CREB and/or AP1.

While there is no specific upper limit for the number TFBS for CREB and/or AP1, in general it is preferred that the CRE comprises 15 or fewer TFBS for CREB and/or AP1, optionally 10 or fewer TFBS for CREB and/or AP1.

In some embodiments the CRE comprises at least 1 TFBS for each of CREB and AP1. In some embodiments the CRE comprises at least 2, 3, 4, or 5 TFBS for each of CREB and AP1.

TFBS for CREB typically comprise or consist of the highly conserved consensus sequence TGACGTCA (SEQ ID NO: 1). This sequence is known as the cAMP Responsive Element (or cAMPRE or CRE; the abbreviation cAMPRE will be used herein to avoid confusion with cis-regulatory element).

TFBS for AP1 typically comprise or consist of the consensus sequence TGA[GC]TCA (SEQ ID NO: 2). In specific examples of the present invention the sequences TGAGTCA (named AP1(1), SEQ ID NO: 3) and TGACTCAG (named AP1(2), SEQ ID NO: 4) were used, and thus AP1(1) and AP1(2) can be viewed as preferred TFBS for AP1. The generic term AP1 in respect of a TFBS refers to a TFBS comprising the above consensus sequence, and it encompasses both AP1(1) and AP1(2).

In some preferred embodiments of the present invention the CRE comprises at least one TFBS for a transcription factor other than CREB and/or AP1. In some preferred embodiments of the present invention the CRE comprises at least one TFBS for ATF6 and/or hypoxia inducible factor (HIF). The CRE may comprise 2, 3, 4, 5, 6, 7, 8, 9, or 10 TFBS for a transcription factor other than CREB and/or AP1, for example for ATF6 and/or HIF. In some embodiments of the invention the CRE comprises at least 1 TFBS for each of ATF6 and HIF, for example 3 TFBS for each of ATF6 and HIF.

TFBS for HIF comprise or consist of the consensus sequence NCGTG (SEQ ID NO: 5), more preferably [AG]CGTG (SEQ ID NO: 6). This sequence is referred to as the HIF binding sequence (HBS). In specific examples of the present invention the HBS sequence CTGCACGTA (named HRE1, SEQ ID NO: 7) was used, and thus HRE1 can be viewed as a preferred TFBS for HIF. However, other TFBS for HIF are known and can be used in the present invention, for example ACGTGC (SEQ ID NO: 8) or ACCTTGAGTACGTGCGTCTCTGCACGTATG (SEQ ID NO: 9).

TFBS for ATF6 comprise or consist of the consensus sequence TGACGT (SEQ ID NO: 10), more preferably TGACGTG (SEQ ID NO: 11). In specific examples of the present invention the TFBS sequence TGACGTGCT (SEQ ID NO: 12) was used and this can be viewed as preferred TFBS for ATF6. However, in general, any sequence comprising the consensus sequence TGACGT (SEQ ID NO: 10), more preferably TGACGTG (SEQ ID NO: 11), can be used.

Each of the TFBS discussed above can be present in either orientation (i.e. they can be functional when present on either strand of the double-stranded DNA). Thus, it will be apparent that any of the TFBS may be represented by the reverse complement consensus sequence in one strand, which indicates the presence of the TFBS sequence on the corresponding complementary strand (in such cases the TFBS can be described as being in the “reverse orientation” or “opposite orientation”). In general, a reference to a TFBS, whether by name or by reciting the sequence of a TFBS, should be considered to refer to the presence of the TFBS in either orientation. When a sequence of a TFBS is recited it should be appreciated that the orientation shown represents a specifically disclosed, and typically a preferred, embodiment.

In some embodiments of the present invention the CRE comprises:
5 TFBS for CREB and 3 TFBS for AP1;
5 TFBS for CREB and 4 TFBS for AP1;
8 TFBS for AP1; or
3 TFBS for ATF6, 4 TFBS for AP1 and 3 TFBS for HIF;
   wherein adjacent TFBS are optionally, but preferably, separated by spacer sequences.

The spacer sequence can be any suitable length. Typically, the spacer is from 2 to 100 nucleotides in length, from 5 to 50 nucleotides in length, from 6 to 40 nucleotides in length, from 7 to 30 nucleotides in length, from 8 to 25 nucleotides in length or from 10 to 20 nucleotides in length. Spacers of 10 and 20 nucleotides in length have been used in some specific embodiments of the invention, and these function well, but other lengths of spacers can be used. In some embodiments it is preferred that the spacer is a multiple of 5 nucleotides in length. The skilled person can readily determine suitable lengths of spacers.

It should be noted that the sequence and length of the of the spacers can vary; that is to say that each spacer in a sequence need not have the same sequence or length as any other. For convenience, some or all of the spacers between TFBS in a CRE often do have the same sequence and length, so, while this may be preferred, it is not required.

The TFBS can suitably be in any order, but in preferred embodiments they are provided in the order in which they are recited, i.e. in the first embodiment in the list above there would be 4 TFBS for cAMPRE and then 3 TFBS for AP1 in an upstream to downstream direction.

In some embodiments of the present invention the CRE consists of:
5 TFBS for CREB and 3 TFBS for AP1;
5 TFBS for CREB and 4 TFBS for AP1;
8 TFBS for AP1; or
3 TFBS for ATF6, 4 TFBS for AP1 and 3 TFBS for HIF;
   wherein adjacent TFBS are optionally, but preferably, separated by spacer sequences. Suitable lengths for the spacers are discussed above.

Again, the TFBS can suitably be in any order, but in preferred embodiments they are provided in the order in which they are recited.

In some embodiments of the present invention the CRE comprises one of the following structures:
cAMPRE-S-cAMPRE-S-cAMPRE-S-cAMPRE-S-cAMPRE-S-AP1-S-AP1-S-AP1 (CRE comprising 5×cAMPRE and 3× AP1 TFBS);
cAMPRE-S-cAMPRE-S-cAMPRE-S-cAMPRE-S-cAMPRE-S-AP1(2)-S-AP1(2)-S-AP1(2) (CRE comprising 5×cAMPRE and 3× AP1(2) TFBS);
cAMPRE-S-cAMPRE-S-cAMPRE-S-cAMPRE-S-cAMPRE-S-AP1-S-AP1-S-AP1-S-AP1 (CRE comprising 5×cAMPRE and 4× AP1 TFBS);
cAMPRE-S-cAMPRE-S-cAMPRE-S-cAMPRE-S-cAMPRE-S-AP1 (2)-S-AP1 (2)-S-AP1 (2)-S-AP1(2) (CRE comprising 5×cAMPRE and 4× AP1(2) TFBS);
AP1-S-AP1-S-AP1-S-AP1-S-AP1-S-AP1-S-AP1-S-AP1 (CRE comprising 8× AP1 TFBS);
AP1(1)-S-AP1(1)-S-AP1(1)-S-AP1(1)-S-AP1(1)-S-AP1(1)-S-AP1(1)-S-AP1(1) (CRE comprising 8× AP1(1) TFBS);
ATF6-S-ATF6-S-ATF6-S-AP1-S-AP1-S-AP1-S-AP1-S-HIF-S-HIF-S-HIF (CRE comprising 3×ATF6, 4× AP1 and 3×HIF TFBS);
ATF6-S-ATF6-S-ATF6-S-AP1(1)-S-AP1 (1)-S-AP1(1)-S-AP1(1)-S-HRE1-S-HRE1-S-HRE1 (CRE comprising 3×ATF6, 4× AP1(1) and 3×HRE1 TFBS);
cAMPRE-S-cAMPRE-S-cAMPRE-S-cAMPRE-S-cAMPRE-S-AP1-S-AP1-S-AP1-S-AP1 (CRE comprising 5×cAMPRE 4× AP1 TFBS); and
cAMPRE-S-cAMPRE-S-cAMPRE-S-cAMPRE-S-cAMPRE-S-AP1(1)-S-AP1(1)-S-AP1(1)-S-AP1(1) (CRE comprising 5×cAMPRE 4× AP1(1) TFBS);
   wherein S represents an optional, but preferable, spacer sequence. Suitable lengths for the spacers are discussed above.

In these structures, reference to a TF represents the presence of the TFBS for that TF. cAMPRE is used to refer to the TFBS for CREB.

In some specific embodiments of the present invention the CRE comprises one of the following structures:
cAMPRE-S₁₀-cAMPRE-S₁₀-cAMPRE-S₁₀-cAMPRE-S₁₀-cAMPRE-S₁₀-AP1-S₁₀-AP1-S₁₀-AP1 (CRE comprising 5×cAMPRE and 3× AP1 TFBS);
cAMPRE-S₁₀-cAMPRE-S₁₀-cAMPRE-S₁₀-cAMPRE-S₁₀-cAMPRE-S₁₀-AP1(2)-S₁₀-AP1 (2)-S₁₀-AP1(2) (CRE comprising 5×cAMPRE and 3× AP1(2) TFBS);
cAMPRE-S₁₀-cAMPRE-S₁₀-cAMPRE-S₁₀-cAMPRE-S₁₀-cAMPRE-S₁₀-AP1-S₁₀-AP1-S₁₀-AP1-S₁₀-AP1 (CRE comprising 5×cAMPRE and 4× AP1 TFBS);
cAMPRE-S₁₀-cAMPRE-S₁₀-cAMPRE-S₁₀-cAMPRE-S₁₀-cAMPRE-S₁₀-AP1(2)-S₁₀-AP1 (2)-S₁₀-AP1(2)-S₁₀-AP1(2) (CRE comprising 5×cAMPRE and 4× AP1(2) TFBS);
AP1-S₂₀-AP1-S₂₀-AP1-S₂₀-AP1-S₂₀-AP1-S₂₀-AP1-S₂₀-AP1-S₂₀-AP1 (CRE comprising 8×AP1 TFBS);
AP1 (1)-S₂₀-AP1 (1)-S₂₀-AP1(1)-S₂₀-AP1(1)-S₂₀-AP1 (1)-S₂₀-AP1 (1)-S₂₀-AP1 (1)-S₂₀-AP1 (1) (CRE comprising 8× AP1(1) TFBS);
ATF6-S₂₀-ATF6-S₂₀-ATF6-S₂₀-AP1-S₂₀-AP1-S₂₀-AP1-S₂₀-AP1-S₂₀-HIF-S₂₀-HIF-S₂₀-HIF (CRE comprising 3×ATF6, 4× AP1 and 3×HIF TFBS); and
ATF6-S₂₀-ATF6-S₂₀-ATF6-S₂₀-AP1(1)-S₂₀-AP1(1)-S₂₀-AP1 (1)-S₂₀-AP1 (1)-S₂₀-HRE1-S₂₀-HRE1-S₂₀-HRE1 (CRE comprising 3×ATF6, 4× AP1(1) and 3×HRE1 TFBS);
cAMPRE-S₁₀-cAMPRE-S₁₀-cAMPRE-S₁₀-cAMPRE-S₁₀-cAMPRE-S₁₀-AP1-S₁₀-AP1-S₁₀-AP1-S₁₀-AP1 (CRE comprising 5×cAMPRE 4× AP1 TFBS); and
cAMPRE-S₁₀-cAMPRE-S₁₀-cAMPRE-S₁₀-cAMPRE-S₁₀-cAMPRE-S₁₀-AP1(1)-S₁₀-AP1 (1)-S₁₀-AP1(1)-S₁₀-AP1(1) (CRE comprising 5×cAMPRE 4× AP1(1) TFBS);
wherein Sₓ represents a spacer sequence of length X nucleotides.

The lengths of spacers specified above have been found to be effective in the specific examples discussed below. While other lengths of spacers are also expected to be functional, these represent preferred spacer lengths; this applies with respect to all aspects and embodiments of the invention comprising these TFBS as set out below.

In some specific embodiments of the present invention the CRE comprises one of the following sequences:

TGACGTCA-S-TGACGTCA-S-TGACGTCA-S-TGACGTCA-S-
TGACGTCA-S-TGA[GC]TCA-S-TGA[GC]TCA-S-TGA[GC]
TCA
(SEQ ID NO: 13, CRE comprising 5× cAMPRE
and 3× AP1 TFBS);
TGACGTCA-S-TGACGTCA-S-TGACGTCA-S-TGACGTCA-S-
TGACGTCA-S-TGA[GC]TCA-S-TGA[GC]TCA-S-TGA[GC]
TCA-S-TGA[GC]TCA
(SEQ ID NO: 14, OREcomprising 5× cAMPRE and
4× AP1 TFBS);
TGA[GC]TCA-S-TGA[GC]TCA-S-TGA[GC]TCA-S-TGA
[GC]TCA-S-TGA[GC]TCA-S-TGA[GC]TCA-S-TGA[GC]
TCA-S-TGA[GC]TCA
(SEQ ID NO: 15, CRE comprising 8× AP1 TFBS);
and
TGACGT-S-TGACGT-S-TGACGT-S-TGA[GC]TCA-S-TGA
[GC]TCA-S-TGA[GC]TCA-S-TGA[GC]TCA-S-[AG]CGT
G-S-[AG]CGTG-S-[AG]CGTG
(SEQ ID NO: 16, CRE 3× ATF6, 4× AP1 and
3× HIFTFBS);

wherein S represents an optional, but preferable, spacer sequence. Suitable lengths for the spacers are discussed above.

In some specific embodiments of the present invention the CRE comprises one of the following sequences:

TGACGTCA-S-TGACGTCA-S-TGACGTCA-S-TGACGTCA-S-
TGACGTCA-S-TGACTCAG-S-TGACTCAG-S-TGACTCAG
(SEQ ID NO: 18, CRE from Synp-FORCSV-10,
comprising 5× cAMPRE and 3× AP1(2) TFBS);
TGACGTCA-S-TGACGTCA-S-TGACGTCA-S-TGACGTCA-S-
TGACGTCA-S-TGACTCAG-S-TGACTCAG-S-TGACTCAG-S-
TGACTCAG
(SEQ ID NO: 19, CRE from Synp-FORCMV-09
comprising 5× cAMPRE and 4×AP1(2) TFBS);
TGAGTCA-S-TGAGTCA-S-TGAGTCA-S-TGAGTCA-S-TGAG
TCA-S-TGAGTCA-S-TGAGTCA-S-TGAGTCA
(SEQ ID NO: 20, CRE from Synp-FMP-02, Synp-
FLP-01 and Synp-RTV-017 comprising 8× AP1(1)
TFBS);
TGACGTGCT-S-TGACGTGCT-S-TGACGTGCT-S-TGAGTCA-
S-TGAGTCA-S-TGAGTCA-S- TGAGTCA-S-CTGCACGTA-S-
CTGCACGTA-S-CTGCACGTA
(SEQ ID NO: 21, CRE from Synp-RTV-019
comprising 3× ATF6, 4× AP1(1) and
3× HRE1 TFBS);
and
TGACGTCA-S-TGACGTCA-S-TGACGTCA-S-TGACGTCA-S-
TGACGTCA-S-TGACTCA-S-TGACTCA-S-TGACTCA-S-TG
ACTCA
(SEQ ID NO: 22, CRE from Synp-FORCYB1
comprising 5× cAMPRE and 4× AP1(1) TFBS);

wherein S represents an optional, but preferable, spacer sequence. Suitable lengths for the spacers are discussed above.

In some specific preferred embodiments of the present invention the CRE comprises one of the following sequences:

TGACGTCACGATTACCATTGACGTCACGATTACCA
TTGACGTCACGATTACCATTGACGTCACGATTACC
ATTGACGTCAGCGATTAAGATGACTCAGCGATTAA
GATGACTCAGCGATTAAGATGACTCAG
(SEQ ID NO: 23, CRE from Synp-FORCSV-10,
comprising 5× cAMPRE and 3×AP1(2) TFBS);
TGACGTCACGATTACCATTGACGTCACGATTACCA
TTGACGTCACGATTACCATTGACGTCACGATTACC
ATTGACGTCAGCGATTAAGATGACTCAGCGATTAA
GATGACTCAGCGATTAAGATGACTCAGCGATTAAG
ATGACTCAG
(SEQ ID NO: 24, CRE from Synp-FORCMV-09
comprising 5×cAMPRE and 4×AP1(2) TFBS);
TGAGTCAGATGATGCGTAGCTAGTAGTTGAGTCAG
ATGATGCGTAGCTAGTAGTTGAGTCAGATGATGCG
TAGCTAGTAGTTGAGTCAGATGATGCGTAGCTAGT
AGTTGAGTCAGATGATGCGTAGCTAGTAGTTGAGT
CAGATGATGCGTAGCTAGTAGTTGAGTCAGATGAT
GCGTAGCTAGTAGTTGAGTCA
(SEQ ID NO: 25, CRE from Synp-FMP-02,
Synp-FLP-01 and Synp-RTV-017 comprising
8× AP1(1) TFBS);
TGACGTGCTGATGATGCGTAGCTAGTAGTTGACGT
GCTGATGATGCGTAGCTAGTAGTTGACGTGCTGAT
GATGCGTAGCTAGTAGTTGAGTCAGATGATGCGTA
GCTAGTAGTTGAGTCAGATGATGCGTAGCTAGTAG
TTGAGTCAGATGATGCGTAGCTAGTAGTTGAGTCA
GATGATGCGTAGCTAGTAGTCTGCACGTAGATGAT
GCGTAGCTAGTAGTCTGCACGTAGATGATGCGTAG
CTAGTAGTCTGCACGTA
(SEQ ID NO: 26, CRE from Synp-RTV-019
comprising 3× ATF6, 4× AP1(1) and
3× HRE1 TFBS);
and
TGACGTCACGATTACCATTGACGTCACGATTACCA
TTGACGTCACGATTACCATTGACGTCACGATTACC
ATTGACGTCAGCGATTAAGATGACTCAGCGATTAA
GATGACTCAGCGATTAAGATGACTCAGCGATTAAG
ATGACTCA
(SEQ ID NO: 27, CRE from Synp-
FORCYB1 comprising 5× cAMPRE and
4× AP1(1) TFBS);

or a functional variant of any of said sequences that comprises a sequence that is at least 80% identical thereto, preferably 85%, 90%, 95% or 99% identical thereto.

Typically, it is preferred that in such functional variants the TFBS sequences present are identical to the reference sequence, and substantially all variation arises in the spacer sequences lying therebetween.

The abovementioned CREs have been shown to provide good levels of inducibility and powerful expression upon induction, and low levels of background expression, when combined with a minimal promoter to for an inducible promoter. Thus, they are all useful for the provision of forskolin inducible promoters. The CREs demonstrate some degree of variation in terms of inducibility and expression levels upon induction, and this allows a promoter to be selected which has desired properties.

A CRE having the following structure cAMPRE-cAMPRE-S-cAMPRE-S-cAMPRE-S-cAMPRE-S-AP1-S-AP1-S-AP1-S-AP1 (and as exemplified by SEQ ID NO: 14, 17, 19, 22, 27 and 34) has been shown to provide excellent properties in terms of inducibility and expression when coupled to a minimal promoter. Accordingly, such a CRE represents a particularly preferred embodiment of the invention.

A CRE having the following structure ATF6-S-ATF6-S-ATF6-S-AP1-S-AP1-S-AP1-S-AP1-S-HIF-S-HIF-S-HIF (and as exemplified by SEQ ID NO: 16, 21 and 26) has been shown to provide exceptional properties in terms of inducibility and expression when coupled to a minimal promoter. Accordingly, such a CRE represents a particularly preferred embodiment of the invention. It is surprising that such a CRE should perform so well, given that it includes several TFBS that are not known or expected to be induced by forskolin, and fewer TFBS that are induced by forskolin than some other CREs that are less inducible and powerful. It seems that an unexpected synergy has arisen in view of the combination of TFBS present in the CRE.

In a second aspect the present invention provides a cis-regulatory module (CRM) comprising a CRE according to the first aspect of the invention. Other CREs in the CRM can be forskolin-inducible CREs, or can have any other function.

In a third aspect the present invention provides a synthetic forskolin-inducible promoter comprising a CRE according to the first aspect of the invention or CRM according to the second aspect of the invention as defined above. Preferably the synthetic inducible promoter comprises the CRE or CRM operably linked to a minimal promoter or a proximal promoter, preferably a minimal promoter.

The minimal promoter can be any suitable minimal promoter. A wide range of minimal promoters are known in the art. Without limitation, suitable minimal promoters include CMV minimal promoter (CMV-MP), YB-TATA minimal promoter (YB-TABA), HSV thymidine kinase minimal promoter (MinTK), SV40 minimal promoter (SV40-MP), or G6PC-MP (which is a liver-derived non-TATA box MP). The minimal promoter can be a synthetic minimal promoter.

The sequence of CMV-MP is:
(SEQ ID NO: 28)
AGGTCTATATAAGCAGAGCTCGTTTAGTGAACCGT
CAGATCGCCTAGATACGCCATCCACGCTGTTTTGA
CCTCCATAGAAGATCGCCACC.
The sequence of YB-TATA is:
(SEQ ID NO: 29)
GCGATTAATCCATATGCTCTAGAGGGTATATAATG
GGGGCCACTAGTCTACTACCAGAAAGCTTGGTACC
GAGCTCGGATCCAGCCACC.

However, a shorter version of the YB-TATA MP is known in the art and this is should provide an effective alternative to the YB-TATA MP sequence recited above. The sequence of this shorter YB-TATA MP (referred to as sYB-TATA) is TCTAGAGGGTATATAATGGGGGCCA (SEQ ID NO: 57). Accordingly, wherever YB-TATA is referred to as a component of an inducible promoter herein, an equivalent sequence with sYB-TATA substituted in place of YB-TATA is also considered to be an alternative embodiment of the invention. In other words, in such an alternative the sequence of sYB-TATA is retained, while the remaining portions of YB-TATA can be replaced with other sequences, typically spacer sequences.

The sequence of the MinTK MP is:
(SEQ ID NO: 30)
TTCGCATATTAAGGTGACGCGTGTGGCCTCGAACA
CCGAGCGACCCTGCAGCGACCCGCTTAA.
The sequence of SV40-MP is:
(SEQ ID NO: 31)
TGCATCTCAATTAGTCAGCAACCATAGTCCCGCCC
CTAACTCCGCCCATCCCGCCCCTAACTCCGCCCAG
TTCCGCCCATTCTCCGCCCCATCGCTGACTAATTT
TTTTTATTTATGCAGAGGCCGAGGCCGCCTCGGCC
TCTGAGCTATTCCAGAAGTAGTGAGGAGGCTTTTT
TGGAGGCCTAGGCTTTTGCAAA.
The sequence of G6PC-MP is:
(SEQ ID NO: 32)
GGGCATATAAAACAGGGGCAAGGCACAGACTCATA
GCAGAGCAATCACCACCAAGCCTGGAATAACTGCA
GCCACC
The sequence of MP1 is:
(SEQ ID NO: 80)
TTGGTACCATCCGGGCCGGCCGCTTAAGCGACGCC
TATAAAAAATAGGTTGCATGCTAGGCCTAGCGCTG
CCAGTCCATCTTCGCTAGCCTGTGCTGCGTCAGTC
CAGCGCTGCGCTGCGTAACGGCCGCC

In preferred embodiments of the present invention, the synthetic forskolin-inducible promoter suitably comprises any one of the CRE sequences set out in the first aspect of the present invention operably linked to a minimal promoter or a proximal promoter, preferably a minimal promoter. The CRE of the first aspect of the invention is preferably coupled to the MP via a spacer, but in some cases, there may be another CRE provided therebetween. The CRE of the first aspect of the invention may also be operably linked to the MP without a spacer.

The spacer sequence between the CRE and the minimal promoter can be of any suitable length. Typically, the spacer is from 10 to 100 nucleotides in length, from 20 to 80 nucleotides in length, or from 30 to 70 nucleotides in length. For example, spacers of 21, 42, 59, 65 and 66 nucleotides in length have been used in specific non-limiting examples of the invention, and these function well. However, other lengths of spacers can be used, and the skilled person can readily determine suitable lengths of spacers.

In some preferred embodiments of the present invention, the synthetic forskolin-inducible promoter suitably comprises one of the following structures:
cAMPRE-S-cAMPRE-S-cAMPRE-S-cAMPRE-S-cAMPRE-S-AP1-S-AP1-S-AP1-S-MP (i.e. a CRE comprising 5×cAMPRE and 3× AP1 TFBS and a MP);
cAMPRE-S-cAMPRE-S-cAMPRE-S-cAMPRE-S-cAMPRE-S-AP1-S-AP1-S-AP1-S-AP1-S-MP (i.e. a CRE comprising 5×cAMPRE and 4× AP1 TFBS and a MP);
AP1-S-AP1-S-AP1-S-AP1-S-AP1-S-AP1-S-AP1-S-AP1-S-MP (i.e. a CRE comprising 8×AP1 TFBS and a MP);
ATF6-S-ATF6-S-ATF6-S-AP1-S-AP1-S-AP1-S-AP1-S-HIF-S-HIF-S-HIF-S-MP (i.e. a CRE from Synp-RTV-019 comprising 3×ATF6, 4× AP1 and 3×HIF TFBS and a MP); and
cAMPRE-S-cAMPRE-S-cAMPRE-S-cAMPRE-S-cAMPRE-S-AP1-S-AP1-S-AP1-S-AP1-S-MP (i.e. a CRE from Synp-FORCYB1 comprising 5×cAMPRE 4× AP1 TFBS and MP); and
wherein S represents an optional, but preferable, spacer sequence and MP represents a minimal promoter. Suitable lengths for the spacers are discussed above.

In a particularly preferred embodiment of the invention, the synthetic forskolin-inducible promoter comprises the following structure ATF6-S-ATF6-S-ATF6-S-AP1-S-AP1-S-AP1-S-AP1-S-HIF-S-HIF-S-HIF-S-MP, wherein S represents an optional, but preferable, spacer sequence and MP represents a minimal promoter. More preferably, the MP is CMV-MP.

In some preferred embodiments of the present invention, the synthetic forskolin-inducible promoter suitably comprises one of the following structures:
cAMPRE-S-cAMPRE-S-cAMPRE-S-cAMPRE-S-cAMPRE-S-AP1-S-AP1-S-AP1-S-SV40-MP (i.e. a CRE comprising 5×cAMPRE and 3× AP1 TFBS and SV40-MP);
cAMPRE-S-cAMPRE-S-cAMPRE-S-cAMPRE-S-cAMPRE-S-AP1-S-AP1-S-AP1-S-AP1-S-CMV-MP (i.e. a CRE comprising 5×cAMPRE and 4× AP1 TFBS and CMV-MP);
AP1-S-AP1-S-AP1-S-AP1-S-AP1-S-AP1-S-AP1-S-AP1-S-(Min-TK or G6PC MP or CMV-MP) (i.e. a CRE comprising 8× AP1 TFBS and Min-TK or G6PC MP or CMV-MP);
ATF6-S-ATF6-S-ATF6-S-AP1-S-AP1-S-AP1-S-AP1-S-HIF-S-HIF-S-HIF-S-CMV-MP (i.e. CRE from Synp-RTV-019 comprising 3×ATF6, 4× AP1 and 3×HIF TFBS and CMV-MP); and
cAMPRE-S-cAMPRE-S-cAMPRE-S-cAMPRE-S-cAMPRE-S-AP1-S-AP1-S-AP1-S-AP1-S-YB-TATA (i.e. a CRE from Synp-FORCYB1 comprising 5×cAMPRE 4× AP1 TFBS and YB-TATA);
wherein S represents an optional, but preferable, spacer sequence. Suitable lengths for the spacers are discussed above.

In preferred embodiments of the present invention, the synthetic forskolin-inducible promoter suitably comprises one of the following sequences:

TGACGTCA-S-TGACGTCA-S-TGACGTCA-S-TG
ACGTCA-S-TGACGTCA-S-TGA[GC]TCA-S-TG
A[GC]TCA-S-TGA[GC]TCA-S-TGCATCTCAA
TTAGTCAGCAACCATAGTCCCGCCCCTAACTCCGC
CCATCCCGCCCCTAACTCCGCCCAGTTCCGCCCAT
TCTCCGCCCCATCGCTGACTAATTTTTTTTATTTA
TGCAGAGGCCGAGGCCGCCTCGGCCTCTGAGCTAT
TCCAGAAGTAGTGAGGAGGCTTTTTTGGAGGCCTA
GGCTTTTGCAAA
(SEQ ID NO: 33, CRE comprising 5×
CAM PRE and 3× AP1 TFBS and SV40-MP);
TGACGTCA-S-TGACGTCA-S-TGACGTCA-S-TG
ACGTCA-S-TGACGTCA-S-TGA[GC]TCA -S-
TGA[GC]TCA-S-TGA[GC]TCA-S-TGA[GC]TC
A-S-AGGTCTATATAAGCAGAGCTCGTTTAGTGA
ACCGTCAGATCGCCTAGATACGCCATCCACGCTGT
TTTGACCTCCATAGAAGATCGCCACC
(SEQ ID NO: 34, CRE comprising 5× cAMPRE
and 4× AP1 TFBS and CMV-MP);
TGA[GC]TCA-S-TGA[GC]TCA-S-TGA[GC]TCA-
S-TGA[GC]TCA-S-TGA[GC]TCA-S-
TGA[GC]TCA-S-TGA[GC]TCA-S-TGA[GC]TCA-S-
(TTCGCATATTAAGGTGACGCGTGTGGCCTCGAACACC
GAGCGACCCTGCAGCGACCCGCTTAA
or
GGGCATATAAAACAGGGGCAAGGCACAGACTCATAGCA
GAGCAATCACCACCAAGCCTGGAATAACTGCAGCCACC
or
AGGTCTATATAAGCAGAGCTCGTTTAGTGAACCGTCAG
ATCGCCTAGATACGCCATCCACGCTGTTTTGACCTCCA
TAGAAGATCGCCACC)
(SEQ ID NO: 35, CRE comprising 8× AP1 TFBS and
Min-TK or G6PC MP or CMV-MP);
TGACGT-S-TGACGT-S-TGACGT-S-TGA[GC]T
CA-S-TGA[GC]TCA-S-TGA[GC]TCA-S-TGA
[GC]TCA-S-[AG]CGTG-S-[AG]CGTG-S-[AG]
CGTG-S-AGGTCTATATAAGCAGAGCTCGTTTA
GTGAACCGTCAGATCGCCTAGATACGCCATCCACG
CTGTTTTGACCTCCATAGAAGATCGCCACC
(SEQ ID NO: 36, CRE 3× ATF6,
4× AP1 and 3× HIF TFBS and CMV-MP);
and
TGACGTCA-S-TGACGTCA-S-TGACGTCA-S-TG
ACGTCA-S-TGACGTCA-S-TGA[GC]TCA-S- T
GA[GC]TCA-S-TGA[GC]TCA-S-TGA[GC]TCA
-S-
GCGATTAATCCATATGCTCTAGAGGGTATAT
AATGGGGGCCACTAGTCTACTACCAGAAAGCTTGG
TACCGAGCTCGGATCCAGCCACC
(SEQ ID NO: 37, CRE comprising 5×
cAMPRE and 4× AP1 TFBS and YB-TATA);

wherein S represents an optional, but preferable, spacer sequence. Suitable lengths for the spacers are discussed above.

In some preferred embodiments of the present invention, the synthetic forskolin-inducible promoter suitably comprises one of the following sequences (the TFBS sequences are underlined and minimal promoter sequences are shown in bold):

(SEQ ID NO: 39)
TGACGTCACGATTACCATTGACGTCACGATTACCATTGACGTCACGATTACCATTGACGTCAC
GATTACCATTGACGTCAGCGATTAAGATGACTCAGCGATTAAGATGACTCAGCGATTAAGA
TGACTCAGCGATTAAGATGACTCACTAGCCCGGGCTCGAGATCTGCGATCTGCATCTCAATTA
GTCAGCAACCATAGTCCCGCCCCTAACTCCGCCCATCCCGCCCCTAACTCCGCCCAGTTC
CGCCCATTCTCCGCCCCATCGCTGACTAATTTTTTTTATTTATGCAGAGGCCGAGGCCGCCT
CGGCCTCTGAGCTATTCCAGAAGTAGTGAGGAGGCTTTTTTGGAGGCCTAGGCTTTTGCAAA;
(SEQ ID NO: 40)
TGACGTCACGATTACCATTGACGTCACGATTACCATTGACGTCACGATTACCATTGACGTCAC
GATTACCATTGACGTCAGCGATTAAGATGACTCAGCGATTAAGATGACTCAGCGATTAAGAT
GACTCAGCGATTAAGATGACTCAGCGATTAATCCATATGCAGGTCTATATAAGCAGAGCTCG
TTTAGTGAACCGTCAGATCGCCTAGATACGCCATCCACGCTGTTTTGACCTCCATAGAAGA
TCGCCACC;
(SEQ ID NO: 41)
TGAGTCAGATGATGCGTAGCTAGTAGTTGAGTCAGATGATGCGTAGCTAGTAGTTGAGTCAG
ATGATGCGTAGCTAGTAGTTGAGTCAGATGATGCGTAGCTAGTAGTTGAGTCAGATGATGCG
TAGCTAGTAGTTGAGTCAGATGATGCGTAGCTAGTAGTTGAGTCAGATGATGCGTAGCTAGT
AGTTGAGTCAGTAGTCGTATGCTGATGCGCAGTTAGCGTAGCTGAGGTACCGTCGACGATAT
CGGATCCTTCGCATATTAAGGTGACGCGTGTGGCCTCGAACACCGAGCGACCCTGCAGCG
ACCCGCTTAA;
(SEQ ID NO: 42)
TGAGTCAGATGATGCGTAGCTAGTAGTTGAGTCAGATGATGCGTAGCTAGTAGTTGAGTCAG
ATGATGCGTAGCTAGTAGTTGAGTCAGATGATGCGTAGCTAGTAGTTGAGTCAGATGATGCG
TAGCTAGTAGTTGAGTCAGATGATGCGTAGCTAGTAGTTGAGTCAGATGATGCGTAGCTAGT
AGTTGAGTCAGTAGTCGTATGCTGATGCGCAGTTAGCGTAGCTGAGGTACCGTCGACGATAT
CGGATCCGGGCATATAAAACAGGGGCAAGGCACAGACTCATAGCAGAGCAATCACCACC
AAGCCTGGAATAACTGCAGCCACC;
(SEQ ID NO: 43)
TGAGTCAGATGATGCGTAGCTAGTAGTTGAGTCAGATGATGCGTAGCTAGTAGTTGAGTCAG
ATGATGCGTAGCTAGTAGTTGAGTCAGATGATGCGTAGCTAGTAGTTGAGTCAGATGATGCG
TAGCTAGTAGTTGAGTCAGATGATGCGTAGCTAGTAGTTGAGTCAGATGATGCGTAGCTAGT
AGTTGAGTCAGTAGTCGTATGCTGATGCGCAGTTAGCGTAGCTGAGGTACCGTCGACGATAT
CGGATCCAGGTCTATATAAGCAGAGCTCGTTTAGTGAACCGTCAGATCGCCTAGATACGCC
ATCCACGCTGTTTTGACCTCCATAGAAGATCGCCACC;
(SEQ ID NO: 44)
TGACGTGCTGATGATGCGTAGCTAGTAGTTGACGTGCTGATGATGCGTAGCTAGTAGTTGAC
GTGCTGATGATGCGTAGCTAGTAGTTGAGTCAGATGATGCGTAGCTAGTAGTTGAGTCAGAT
GATGCGTAGCTAGTAGTTGAGTCAGATGATGCGTAGCTAGTAGTTGAGTCAGATGATGCGTA
GCTAGTAGTCTGCACGTAGATGATGCGTAGCTAGTAGTCTGCACGTAGATGATGCGTAGCTA
GTAGTCTGCACGTAGATGATGCGTAGCTAGTAGTGCAGTTAGCGTAGCTGAGGTACCGTCG
ACGATATCGGATCCAGGTCTATATAAGCAGAGCTCGTTTAGTGAACCGTCAGATCGCCTAG
ATACGCCAICCACGCTGTTTTGACCTCCATAGAAGATCGCCACC;
and
(SEQ ID NO: 45)
TGACGTCACGATTACCATTGACGTCACGATTACCATTGACGTCACGATTACCATTGACGTCAC
GATTACCATTGACGTCAGCGATTAAGATGACTCAGCGATTAAGATGACTCAGCGATTAAGAT
GACTCAGCGATTAAGATGACTCAGCGATTAATCCATATGCTCTAGAGGGTATATAATGGGG
GCCACTAGTCTACTACCAGAAAGCTTGGTACCGAGCTCGGATCCAGCCACC

or a functional variant of any of said sequences that comprises a sequence that is at least 80% identical thereto, preferably 85%, 90%, 95% or 99% identical thereto.

Typically, it is preferred that in such functional variants the TFBS and MP sequences present are identical to the reference sequence, and substantially all sequence variation arises in the spacer sequences lying therebetween.

The abovementioned forskolin-inducible promoters have been shown to provide good levels of inducibility and powerful expression upon induction, and low levels of background expression. The promoters demonstrate a degree of variation in terms of inducibility and expression levels upon induction, and this allows a promoter to be selected which has desired properties.

A synthetic forskolin-inducible promoter comprising the structure ATF6-S-ATF6-S-ATF6-S-AP1-S-AP1-S-AP1-S-AP1-S-HIF-S-HIF-S-HIF-S-MP, preferably wherein the MP is CMV-MP, as specifically exemplified in SEQ ID NO: 44, has been shown to provide exceptional properties in terms of inducibility and expression. Accordingly, such a promoter represents a particularly preferred embodiment of the invention. As mentioned above, it is surprising that such a CRE should perform so well, given that it includes several TFBS that are not known or expected to be induced by forskolin, and fewer TFBS that are induced by forskolin than some other CREs that are less inducible and powerful. It seems that an unexpected synergy has arisen in view of the combination of TFBS present in the CRE. In one particularly preferred embodiment of the present invention, the synthetic forskolin-inducible promoter comprises the sequence TGACGT-S₂₀-TGACGT-S₂₀-TGACGT-S₂₀-TGA[GC]TCA-S₂₀-TGA[GC]TCA-S₂₀-TGA[GC]TCA-S₂₀-TGA[GC]TCA-S₂₀-[AG]CGTG-S₂₀-[AG]CGTG-S₂₀-[AG]CGTG-S₆₁-CMV-MP (SEQ ID NO: 38). Herein, Sₓ represents a spacer of length X nucleotides.

In preferred embodiments of the invention, the inducibility of the promoter is such that upon induction (e.g. after cells, e.g. CHO-K1SV cells, are exposed to 18 μM forskolin for 5 h) the expression level of the transgene which is under the control of the promoter is increased by at least a 3-fold, more preferably a 5-, 10-, 15-, 20-, 30-, or 50-fold.

In preferred embodiments of the invention, upon induction (e.g. after cells, e.g. CHO-K1SV cells, are exposed to 18 μM forskolin for 5 h) the expression level of the transgene which is under the control of the promoter is at least 50% of that provided by the CMV-IE promoter (i.e. an otherwise identical vector in the same cells under the same conditions, but in which expression of the transgene is under control of CMV-IE rather than the forskolin inducible promoter). More preferably the expression level of the transgene is at least 75%, 100%, 150%, 200%, 300%, 400%, 500%, 750% or 1000% of that provided by the CMV-IE promoter.

In a fourth aspect there is provided an expression cassette comprising a CRE according to the first aspect of the present invention, CRM according to the second aspect of the present invention or a promoter according to the third aspect of the present invention operably linked to a transgene.

The transgene typically encodes a product of interest, which may be a protein of interest or polypeptide of interest. The protein of interest or polypeptide of interest can be a protein, a polypeptide, a peptide, a fusion protein, all of which can be expressed in a host cell and optionally secreted therefrom.

Proteins or polypeptides of interest can be, for example, antibodies, enzymes or fragments thereof, cytokines, lymphokines, adhesion molecules, receptors and derivatives or fragments thereof, protein antibiotics, toxin fusion proteins, carbohydrate-protein conjugates, structural proteins, regulatory proteins, vaccines and vaccine like proteins or particles, process enzymes, growth factors, hormones, and any other polypeptides that can serve as agonists or antagonists and/or have therapeutic or diagnostic use. According to a specifically preferred embodiment, the recombinant protein is an immunoglobulin, preferably an antibody or antibody fragment, most preferably a Fab or scFv antibody.

Preferred proteins or polypeptides of interest are therapeutic proteins or polypeptides.

Proteins or polypeptides of particular interest include, for example, but are not limited to, insulin, insulin-like growth factor, hGH, tPA, cytokines, such as interleukins (IL), e.g. IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosis factor (TNF), such as TNF alpha and TNF beta, TNF gamma, TRAIL; G-CSF, GM-CSF, M-CSF, MCP-1, VEGF, afamin (AFM), a1-antitrypsin, α-galactosidase A, α-L-iduronidase, ATP7b, ornithine transcarbamylase, phenylalanine hydroxylase, aromatic amino acid decarboxylase (AADC), ATPase Sarcoplasmic/Endoplasmic Reticulum Ca2+ Transporting 2 (ATP2A2), cystic fibrosis transmembrane conductance regulator (CTFR), glutamic acid decarboxylase 65 kDa protein (GAD65), glutamic acid decarboxylase 67 kDa protein (GAD67), lipoprotein lipase (LPL), nerve growth factor (NGF), neurturin (NTN), porphobilinogen deaminase (PBGD), sarcoglycan alpha (SGCA), soluble fms-like tyrosine kinase-1 (sFLT-1), apoliproteins, low-density lipoprotein receptor (LDL-R), albumin, glucose-6-phosphatase, antibodies, nanobodies, aptamers, anti-viral dominant-negative proteins, and functional fragments, subunits or mutants thereof. Also included is the production of erythropoietin or any other hormone growth factors and any other polypeptides that can serve as agonists or antagonists and/or have therapeutic or diagnostic use. Particularly preferred proteins of interest include antibodies, such as monoclonal, polyclonal, multispecific and single chain antibodies, or fragments thereof, e.g. Fab, Fab′, F(ab′)₂, Fc and Fc′-fragments, heavy and light immunoglobulin chains and their constant, variable or hypervariable region as well as Fv- and Fd-fragments. Preferably the protein of interest is a primate protein, more preferably a human protein.

In further embodiments, the protein of interest is, e.g., BOTOX, yobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-nl, DL-8234, interferon, Suntory (gamma-1 a), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alpha, collagenase, carperitide, recombinant human epidermal growth factor (topical gel, wound healing), DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphanate, octocog alpha, Factor VIII, palifermin, indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (subcutaneous implant, Hydron), deslorelin, histrelin, nafarelin, leuprolide sustained release depot (ATRIGEL), leuprolide implant (DUROS), goserelin, Eutropin, KP-102 program, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex' recombinant human luteinizing hormone, dornase alpha, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alpha, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21 S, vapreotide, idursulfase, omnapatrilat, recombinant serum albumin, certolizumab pegol, glucarpidase, human recombinant C1 esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alpha-n3 (oral), belatacept, transdermal insulin patches, AMG-531, MBP-8298, Xerecept, opebacan, AIDS VAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMID, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 131 I-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, ACM-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutaneous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alpha-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4×, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague FIV vaccine, AG-702, OxSODrol, rBetVI, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111 In-hEGF, AE-37, trasnizumab-DM1, antagonist G, IL-12 (recombinant), PM-02734, IMP-321, rhIGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-1 1031, peptide YY [3-36] (obesity, intranasal), FGLL, atacicept, BR3-Fc, BN-003, BA-058, human parathyroid hormone 1-34 (nasal, osteoporosis), F-18-CCR1, AT-1 100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-1 14, JR-013, Factor XIII, aminocandin, PN-951, 716155, SUN-E7001, TH-0318, BAY-73-7977, tevereiix (immediate release), EP-51216, hGH (controlled release, Biosphere), OGP-I, sifuvirtide, TV4710, ALG-889, Org-41259, rhCCI O, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, or TP-9201.

The product of interest (P01) may also be a nucleic acid, for example an RNA, for example an antisense RNA, microRNA, siRNA, tRNA, rRNAs, or any other regulatory, therapeutic or otherwise useful RNA. Various therapeutic siRNAs have been described in the art, and, by way of non-limiting example, the siRNA may be on that is intended to treat to treat FTDP-17 (frontotemporal dementia), DYT1 dystonia, growth hormone deficiency, BACE1 in Alzheimer's, Leukaemia (e.g. targeting c-raf, bcl-2), melanoma (e.g. targeting ATF2, BRAF), prostate cancer (e.g. targeting P110B), and pancreatic carcinoma (e.g. targeting K-Ras). SiRNA therapies are summarised in “Therapeutic potentials of short interfering RNAs”, Appl Microbiol Biotechnol, DOI 10.1007/s00253-017-8433-z. Similarly, for miRNA, various miRNA therapeutic approaches that could be implemented according to the present invention are summarised in “MicroRNA therapeutics: towards a new era for the management of cancer and other diseases”, Nature Reviews Drug Discovery; 16, 203-222 (2017).

In some embodiments of the invention, the transgene can be useful for gene editing, e.g. a gene encoding a site-specific nuclease, such as a meganuclease, zinc finger nuclease (ZFN), transcription activator-like effector-based nuclease (TALEN), or the clustered regularly interspaced short palindromic repeats system (CRISPR-Cas). Suitably the site-specific nuclease is adapted to edit a desired target genomic locus by making a cut (typically a site-specific double-strand break) which is then repaired via non-homologous end-joining (NHEJ) or homology dependent repair (HDR), resulting in a desired edit. The edit can be the partial or complete repair of a gene that is dysfunctional, or the knock-down or knock-out of a functional gene.

In a fifth aspect there is provided a vector comprising a CRE according to the first aspect of the present invention, CRM according to a second aspect of the present invention, a promoter according to the third aspect of the present invention or expression cassette of the forth aspect of the present invention.

The vector can be any naturally occurring or synthetically generated constructs suitable for uptake, proliferation, expression or transmission of nucleic acids in a cell, e.g. plasmids, minicircles, phagemids, cosmids, artificial chromosomes/mini-chromosomes, bacteriophages, viruses such as baculovirus, retrovirus, adenovirus, adeno-associated virus (AAV), herpes simplex virus, or bacteriophages. Methods for the construction of vectors are well known to the person skilled in the art, and they are described in various publications and reference texts. In particular, techniques for constructing suitable vectors, including a description of the functional and regulatory components such as promoters, enhancers, termination and polyadenylation signals, selection markers, origins of replication, and splicing signals, are known to the person skilled in the art. In preferred embodiments, the vector may be a eukaryotic expression vector. Eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operably linked, are well known in the art and several are commercially available from companies such as Stratagene, La Jolla, Calif.; Invitrogen, Carlsbad, Calif.; and Promega, Madison, Wis.

In some embodiments of the invention, the vector is an expression vector for expression in eukaryotic cells. Examples of eukaryotic expression vectors include, but are not limited to, pW-LNEO, pSV2CAT, pOG44, pXTI and pSG available from Stratagene; pSVK3, pBPV, pMSG and pSVL available from Amersham Pharmacia Biotech; and pCMVDsRed2-express, pIRES2-DsRed2, pDsRed2-Mito, pCMV-EGFP available from Clontech. Many other vectors are well-known and commercially available. For mammalian cells adenoviral vectors, the pSV and the pCMV series of vectors are particularly well-known non-limiting examples. There are many well-known yeast expression vectors including, without limitation, yeast integrative plasmids (YIp) and yeast replicative plasmids (YRp). For plants the Ti plasmid of Agrobacterium is an exemplary expression vector, and plant viruses also provide suitable expression vectors, e.g. tobacco mosaic virus (TMV), potato virus X, and cowpea mosaic virus.

In some embodiments of the invention, the vector is a plasmid. Such a plasmid may include a variety of other functional nucleic acid sequences, such as one or more selectable markers, one or more origins of replication, polycloning sites and the like.

In some embodiments of the invention the vector is episomal or it may be integrated into the genome of a cell.

In a sixth aspect, there is provided a bioprocessing vector comprising an expression cassette, the expression cassette comprising a synthetic hypoxia-inducible promoter operably liked to a transgene, the synthetic hypoxia-inducible promoter comprising at least one hypoxia-responsive element (HRE) that is capable of being bound and activated by a hypoxia-inducible factor (HIF).

HIF is a family of transcription factors which are activated by decrease in the oxygen level in a cell. Under normal oxygen conditions, HIF is degraded following hydroxylation. Hypoxic conditions stabilise HIF and prevent its degradation. This allows HIF to translocate to the nucleus, bind to the HRE and activate HRE-responsive genes.

The hypoxia-inducible promoter typically comprises an HRE that is capable of being bound and activated by HIF operably linked to a minimal promoter. However, in some cases the HRE that is capable of being bound and activated by HIF is operably linked to a promoter other than a minimal promoter (e.g. a proximal promoter, such as a tissue-specific proximal promoter). The particular promoter associated with the HRE can be selected depending on the circumstances, but typically minimal promoters are preferred, especially when it is desired to minimise background expression levels.

HREs are generally composed of multimers of short conserved sequences, termed HIF-binding sites (HBSs). As the name suggests, HBSs are bound by HIF, whereupon the HRE is activated to drive transcription. Accordingly, the HRE of the present invention comprises a plurality of HBS, preferably 3 or more HBS, more preferably from 3 to 10 HBS, more preferably from 3 to 8 HBS, more preferably from 4 to 8 HBS. In some preferred embodiments of the present invention the HRE comprises 5, 6 or 8 HBS.

A core consensus sequence for the HBS has been determined. The core consensus sequence is NCGTG (SEQ ID NO: 5, N represents any nucleotide). There is an indication that A or G is optimal in the first position, so a generally preferred consensus sequence is [AG]CGTG (SEQ ID NO: 6). It should be noted that the HBS is functional when it is present in either strand of the double-stranded DNA (i.e. in either orientation). Accordingly, for example, the HBS may be represented by the reverse complement consensus sequence CACG[CT] (SEQ ID NO: 58) in one strand, indicating the presence of the sequence [AG]CGTG (SEQ ID NO: 6) on the corresponding complementary strand (in such cases the HBS can be described as being in the “reverse orientation” or “opposite orientation”).

The HBSs contained in the HRE of the present invention each preferably comprise the consensus sequence NCGTG (SEQ ID NO: 5), and optionally the consensus sequence [AG]CGTG (SEQ ID NO: 6). Additional sequences flanking the consensus sequence may be present, and these have some effect on the affinity of the HIF for the HBS. Preferred HBS for some embodiments of the invention are discussed below.

Adjacent HBSs are typically, but not always, separated by spacer sequences. The spacing between HBSs in an HRE can have a significant effect on the inducibility and/or overall power of the promoter. In some cases, it may be desirable to optimise spacing between adjacent HBSs in order to maximise inducibility and power of the promoter. In other cases, it may be desirable to use suboptimal spacing in order to provide a promoter with lower inducibility and/or overall power of the promoter. Specific spacing between HBSs present in preferred embodiment of the invention will be discussed below. However, in general, it is typically preferred that the spacing between adjacent core consensus sequences in adjacent HBSs is from 3 to 50 nucleotides. To contribute to high levels of expression, it is typically preferred that the spacing between core consensus sequences in adjacent HBSs is from 7 to 25 nucleotides, preferably about 8 to 22 nucleotides. For intermediate levels of expression, it is typically preferred that the spacing between core consensus sequences in adjacent HBSs is from 5 to 6 nucleotides or from 26 to 32 nucleotides. For low levels of expression, it is typically preferred that the spacing between core consensus sequences in adjacent HBSs is from 2 to 4 nucleotides or from 33 to 50 nucleotides. It will be appreciated that there is scope to vary the spacing between adjacent HBS and thereby tailor the properties of the HRE.

The HRE is typically spaced from the promoter (e.g. minimal promoter), though it need not be. The spacing can have an effect on the inducibility and/or overall power of the promoter. Generally, it is preferred that the spacing between the core consensus sequences in the final HBS (i.e. that which is most proximal to the minimal promoter) and the TATA box (or equivalent sequence if a TATA box is not present) of the minimal promoter is from 0 to 200 nucleotides, more preferably 10 to 100 nucleotides, yet more preferably 20 to 70 nucleotides, yet more preferably 20 to 50 nucleotides, and yet more preferably 20 to 30 nucleotides. To contribute to high levels of expression, it is typically preferred that the spacing between final HBS and the TATA box (or equivalent sequence if a TATA box is not present) of the minimal promoter is 20-30, with spacings significantly over and under this leading to weaker expression levels. It will be appreciated that there is scope to vary the spacing between the final HBS and the MP and thereby tailor the properties of the HRE.

In some embodiments of the present invention, the HRE that is capable of being bound and activated by HIF comprises at least one HBS that comprises or consists of the HRE1 sequence. The HRE1 HBS sequence is ACGTGC (SEQ ID NO: 8). HRE1 of course may be present on either strand of the nucleic acid, and thus in such cases the reverse orientation HRE1 will be indicated by the presence of the reverse complement sequence GCACGT (SEQ ID NO: 59).

In some embodiments of the invention all HBS present in the HRE comprise or consist of the HRE1 sequence. The HRE1 sequences that are present in the HRE may each independently be present in either orientation. In some embodiments it is preferred that all of the HRE1 sequences that are present in the HRE are in the same orientation.

In some embodiments of the present invention, the HRE that is capable of being bound and activated by HIF comprises at least one HBS that comprises or consists of the HRE2 sequence. The sequence of HRE2 is CTGCACGTA (SEQ ID NO: 7). In HRE2 the HBS is present in the reverse orientation when compared with HRE1, and as such the HRE2 sequence contains the reverse complement of the HRE1 sequence. HRE2 may be present on either strand of the nucleic acid, and thus in such cases the reverse orientation HRE2 may be indicated by the presence of the reverse complement sequence TACGTGCAG (SEQ ID NO: 60).

The HRE2 sequence comprises additional flanking sequences and is considered to be an optimised HBS, which binds HIF more strongly than HRE1. Thus, in cases where a high level of promoter inducibility and power are desired, HRE2 may be considered to be preferable to HRE1.

In some embodiments of the invention all of the HBS present in the HRE comprise or consist of the HRE2 sequence. As the HRE2 sequence effectively comprises the HRE1 sequence, it will be apparent that when the HRE2 is provided HRE1 will inevitably also be present. The HRE2 sequences that are present in the HRE may each independently be present in either orientation. In some embodiments it is preferred that all of the HRE2 sequences that are present in the HRE are in the same orientation.

In some embodiments of the present invention, the HRE that is capable of being bound and activated by a HIF comprises at least one HBS that comprises or consists of the HRE3 sequence, or a functional variant thereof.

The HRE3 sequence is ACCTTGAGTACGTGCGTCTCTGCACGTATG (SEQ ID NO: 9, HBS underlined). HRE3 represents a composite HBS which comprises two individual HBSs (i.e. binding sites for HIF, underlined) separated by a spacer, and with further spacers at each end. It can be seen that HRE3 comprises one HBS in each orientation (one comprising HRE1 and one comprising HRE2, the HRE1 sequence being positioned 5′ with respect to the HRE2 sequence). Given that each HRE3 sequence comprises 2 individual HBS, for the purposes of the present invention, each HRE3 sequence or functional variant thereof contributes 2 individual HBS to the total number of HBS present in the HRE.

HRE3 or functional variants thereof may be present on either strand of the nucleic acid, and thus in such cases the reverse orientation of HRE3 may be indicated by the presence of the reverse complement sequence CATACGTGCAGAGACGCACGTACTCAAGGT (SEQ ID NO: 61).

As mentioned above, functional variants of HRE3 also form embodiments of the present invention. Such variants are functional if they retain the ability to be bound by HIF leading to activation. Preferred functional variants of HRE3 retain the same HBSs as HRE3 in substantially the same position and orientation, but contain different spacer sequences. Accordingly, in some preferred embodiments the functional variant of HRE3 suitably has the following sequence:

(SEQ ID NO: 62)
S₁-ACGTG-S₂-CTGCACGTA-S₃;

where S₁ is a spacer of length 8-10, preferably 9,
where S₂ is a spacer of length 4-6, preferably 5,
where S₃ is a spacer of length 1-3, preferably 2.

In some embodiments of the invention, the functional variant of HRE3 comprises the sequence NNNNNNNNNACGTGNNNNNCTGCACGTANN (SEQ ID NO: 63).

In some preferred embodiments, the functional variant of HRE3 has an overall sequence identity to HRE3 of a least 80%, preferably at least 90%, more preferably at least 95% identical to HRE3, and wherein the HBS sequences are completely identical to HRE3.

HRE3 is considered to be a particularly optimal sequence, which binds HIF strongly. Thus, in cases where a high level of promoter inducibility and power are desired, the presence of HRE3 or functional variants thereof that maintain similar properties may be considered to be preferable.

In some embodiments of the invention all HBS present in the HRE comprise or consist of the HRE3 sequence, or a functional variant thereof. The HRE3 sequences, or functional variants thereof, that are present in the HRE may each independently be present in either orientation. In some embodiments it is preferred that all of the HRE3 sequences, or functional variants thereof, that are present in the HRE are in the same orientation.

In some embodiments of the invention, the HRE may comprise a combination of two or more of HRE1, HRE2 and/or HRE3.

In some embodiments of the present invention, the HRE that is capable of being bound and activated by HIF suitably comprises the following sequence:

(SEQ ID NO: 64)
-[ACGTGC-S]ₙ-ACGTGC;

wherein S is a spacer and n is from 2 to 9, preferably from 3 to 7. It should be noted that the sequence of the spacer can vary; that is to say that the spacer in each repeat unit [ACGTGC-S]ₙ (SEQ ID NO: 65) may or may not have the same sequence or length.

The length of the spacer can be varied depending on the desired inducibility and power of the promoter.

Accordingly, in embodiments where is desired to maximise inducibility and power of the promoter, spacers are provided such that spacing between core consensus sequences in adjacent HBSs is from 7 to 18 nucleotides, preferably about 8-12 nucleotides, more preferably about 10 nucleotides. While it is often desirable to maximise inducibility and power of the promoter, in some cases a lower level of inducibility and power may be desired. In embodiments where a somewhat lower level of inducibility and power is desired, spacers can be provided such that adjacent HBSs are spaced apart by lesser or greater amounts, e.g. by from 4-6 nucleotides or from 19-50 nucleotides. It will be apparent that the HRE1 HBS comprises one nucleotide flanking the core consensus sequence (underlined—ACGTGC, SEQ ID NO: 8), and as such the spacers in in these embodiments take this into account to provide the desired spacing.

In some embodiments of the present invention, the HRE that is capable of being bound and activated by HIF suitably comprises the following sequence:

(SEQ ID NO: 66)
ACGTGC-S-ACGTGC-S-ACGTGC-S-ACGTGC-S-ACGTGC

wherein S is a spacer. Suitable lengths for the spacer are discussed above. In some embodiments of the invention the spacers each have a length of 30-50, preferably 40 nucleotides. In such a case, an exemplary, but non-limiting, spacer has the following sequence:

(SEQ ID NO: 67)
GATGATGCGTAGCTAGTAGTGATGATGCGTAGCTAGTAGT.

In one embodiment of the invention the HRE that is capable of being bound and activated by HIF suitably comprises the following sequence: ACGTGCGATGATGCGTAGCTAGTAGTGATGATGCGTAGCTAGTAGTACGTGCGATGAT GCGTAGCTAGTAGTGATGATGCGTAGCTAGTAGTACGTGCGATGATGCGTAGCTAGTA GTGATGATGCGTAGCTAGTAGTACGTGCGATGATGCGTAGCTAGTAGTGATGATGCGT AGCTAGTAGTACGTGC (SEQ ID NO: 68, HBSs underlined), or a functional variant that is that is at least 80% identical thereto, preferably 85%, 90%, 95% or 99% identical thereto. Typically, it is preferred that in such functional variants the HRE1 sequences are substantially or completely identical to the reference sequence, and substantially all sequence variation arises in the spacer sequences.

Such an HRE generally displays a very low level of inducibility and low expression levels when induced. This may be desirable in situations where background expression is to be minimised, and a high level of expression when induced is not required. Optimised spacing of the HBS can of course lead to higher levels of inducibility and expression upon induction.

In some preferred embodiments of the invention the HRE that is capable of being bound and activated by HIF suitably comprises the following sequence:

(SEQ ID NO: 69)
[CTGCACGTA-S]ₙ-CTGCACGTA;

wherein S is an optional spacer and n is from 2 to 9, preferably from 3 to 7. It should be noted that the sequence of the spacer, when present, can vary; that is to say that the spacer in each repeat unit [CTGCACGTA-S]ₙ (SEQ ID NO: 70) may or may not have the same sequence or length.

Details of the suitable spacings between core consensus sequences in adjacent HBSs are discussed above for the preceding embodiments, and these considerations apply to these embodiments equally. It will be apparent that the HRE2 HBS comprises four nucleotides flanking the core consensus sequence (underlined—CTGCACGTA, SEQ ID NO: 7), and as such the spacers in these embodiments take this into account to provide the desired spacing.

In some embodiments of the present invention, the HRE that is capable of being bound and activated by HIF suitably comprises the following sequence: CTGCACGTA-S-CTGCACGTA-S-CTGCACGTA-S-CTGCACGTA-S-CTGCACGTA-S-CTGCACGTA (SEQ ID NO: 71); wherein S is a spacer. Suitable lengths for the spacer are discussed above.

In some embodiments of the invention the spacers each have a length of 20 nucleotides. In such a case, an exemplary, but non-limiting, spacer has the following sequence:

(SEQ ID NO: 72)
GATGATGCGTAGCTAGTAGT.

In one embodiment of the invention the HRE that is capable of being bound and activated by HIF suitably comprises the following sequence: CTGCACGTAGATGATGCGTAGCTAGTAGTCTGCACGTAGATGATGCGTAGCTAGTAGT CTGCACGTAGATGATGCGTAGCTAGTAGTCTGCACGTAGATGATGCGTAGCTAGTAGT CTGCACGTAGATGATGCGTAGCTAGTAGTCTGCACGTA (SEQ ID NO: 73, HBSs underlined), or a functional variant that comprises a sequence that is at least 80% identical thereto, preferably 85%, 90%, 95% or 99% identical thereto. Typically, it is preferred that in such functional variants the HRE2 sequences are substantially or completely identical to the reference sequence, and substantially all sequence variation arises in the spacer sequences. Such an HRE generally displays an intermediate level of inducibility and low expression levels when induced. This may be desirable in situations where background expression is to be minimised, and a moderate level of expression when induced is required. Further optimisation of the spacing of the HBSs can of course lead to higher levels of inducibility and expression upon induction. Likewise, de-optimisation can lead to lower levels of inducibility and expression upon induction.

In some embodiments of the present invention, the HRE that is capable of being bound and activated by HIF suitably comprises the following sequence: CTGCACGTACTGCACGTACTGCACGTACTGCACGTA (SEQ ID NO: 74, HBSs underlined), or a functional variant that comprises a sequence that is at least 80% identical thereto, preferably 85%, 90%, 95% or 99% identical thereto. It can be seen that this HRE comprises no additional spacers between adjacent HRE2 elements. However, in view of the four flanking nucleotides surrounding the core consensus sequence of HRE2, the core consensus sequences have an effective spacing of 4 nucleotides.

Such an HRE generally displays an intermediate level of inducibility and low expression levels when induced. This may be desirable in situations where background expression is to be minimised, and a moderate level of expression when induced required. Further optimisation of the spacing of the HBSs can of course lead to higher levels of inducibility and expression upon induction. Likewise, de-optimisation can lead to lower levels of inducibility and expression upon induction.

In some preferred embodiments of the present invention, the HRE that is capable of being bound and activated by HIF suitably comprises from 3 to 6, preferably from 3 to 5, preferably 4 HRE3 sequences, or a functional variants thereof, wherein adjacent HRE3 sequences, or functional variants thereof, are separated from each other by a spacer having a length of from 4 to 20 nucleotides, preferably from 6 to 15 nucleotides, more preferably 9 nucleotides.

In a preferred embodiment of the present invention, the HRE that is capable of being bound and activated by HIF suitably comprises the following sequence:

(SEQ ID NO: 75)
[ACCTTGAGTACGTGCGTCTCTGCACGTATG-S]ₙ-
ACCTTGAGTACGTGCGTCTCTGCACGTATG;

wherein S is an optional spacer and n is from 2 to 5, preferably from 2 to 4, preferably 3. It should be noted that the sequence of the spacer, if present, can vary; that is to say that the spacer in each repeat unit [ACCTTGAGTACGTGCGTCTCTGCACGTATG-S]ₙ (SEQ ID NO: 76) may or may not have the same sequence or length. It will be appreciated that in the HRE3 sequence as set out above can, in some or all instances, may be replaced with a functional variant thereof.

Details of the suitable spacings between core consensus sequences in adjacent HBSs are discussed above for the preceding embodiments, and these considerations apply to these embodiments equally. It will be apparent that the HRE3 composite HBS comprises 11 nucleotides flanking the region containing the two core consensus sequence (underlined—ACCTTGAGTACGTGCGTCTCTGCACGTATG, SEQ ID NO: 9), and as such the spacers in in these embodiments take this into account to provide the desired spacing. In some embodiments the spacer, S, suitably has a length of from 4 to 20 nucleotides, preferably from 7 to 15 nucleotides, more preferably 9 nucleotides.

In some embodiments of the present invention, the HRE that is capable of being bound and activated by HIF suitably comprises the following sequence:

(SEQ ID NO: 77)
ACCTTGAGTACGTGCGTCTCTGCACGTATG-S-
ACCTTGAGTACGTGCGTCTCTGCACGTATG-S-
ACCTTGAGTACGTGCGTCTCTGCACGTATG-S-
ACCTTGAGTACGTGCGTCTCTGCACGTATG-S-
ACCTTGAGTACGTGCGTCTCTGCACGTATG-S;;

wherein S is a spacer. Suitable lengths for the spacer are discussed above. It will be appreciated that in the HRE3 sequence as set out here can, in some or all instances, be replaced with a functional variant thereof.

In some embodiments of the invention the spacers each have a length of 9 nucleotides. In such a case, an exemplary, but non-limiting, spacer has the following sequence: GCGATTAAG (SEQ ID NO: 78).

In one preferred embodiment of the invention the HRE that is capable of being bound and activated by HIF suitably comprises the following sequence: ACCTTGAGTACGTGCGTCTCTGCACGTATGGCGATTAAGACCTTGAGTACGTGCGTCTC TGCACGTATGGCGATTAAGACCTTGAGTACGTGCGTCTCTGCACGTATGGCGATTAAGA CCTTGAGTACGTGCGTCTCTGCACGTATG (SEQ ID NO: 79, HBSs underlined), or a functional variant that comprises a sequence that is at least 80% identical thereto, preferably 85%, 90%, 95% or 99% identical thereto. Typically, it is preferred that in such functional variants the HRE1 and HRE2 sequences present in the HRE3 sequence are substantially or completely identical to the reference sequence, and substantially all sequence variation arises in the spacer sequences.

Such an HRE generally displays a high level of inducibility and high expression levels when induced. This may be desirable in situations where a high level of expression when induced is required. Further optimisation of the spacing of the HBSs may potentially lead to higher levels of inducibility and expression upon induction. Likewise, de-optimisation can lead to lower levels of inducibility and expression upon induction.

As mentioned previously, the hypoxia-inducible promoter typically comprises the HRE that is capable of being bound and activated by HIF operably linked to a minimal or proximal promoter. It is preferred that the prompter operably linked to the HIF is a minimal promoter.

The minimal promoter can be any suitable minimal promoter. A wide range of minimal promoters are known in the art. Without limitation, suitable minimal promoters include CMV minimal promoter (CMV-MP), YB-TATA minimal promoter (YB-TABA), HSV thymidine kinase minimal promoter (MinTK), and SV40 minimal promoter (SV40-MP). The minimal promoter can be a synthetic minimal promoter. Particularly preferred minimal promoters are the CMV minimal promoter (CMV-MP) and YB-TATA minimal promoter (YB-TABA). Suitable minimal promoters are presented hereinabove in relation to the third aspect of the present invention. Suitably, the hypoxia-inducible promoter typically comprises the HRE that is capable of being bound and activated by HIF operably linked to a minimal promoter according any one of SEQ ID NOs: 28-32, 57 and 80.

Accordingly, preferred embodiments of the present invention comprise the HRE that is capable of being bound and activated by HIF operably linked to one of the abovementioned minimal promoters, more preferably to CMV-MP or YB-TATA, and most preferably CMV-MP. CMV-MP, when combined with HREs of the present invention, has shown to provide extremely high levels of inducibility and high promoter strength. Low background expression levels have also been observed.

The HRE is preferably spaced from the minimal promoter (or other type of promoter, if used) by a spacer sequence. The spacing between the HRE and the minimal promoter can affect the inducibility and power of the hypoxia-inducible promoter. Generally, it is preferred that the spacing between the core consensus sequences in the final HBS (i.e. that which is most proximal to the minimal promoter) and the TATA box (or equivalent sequence if a TATA box is not present) of the minimal promoter is from 10 to 100 nucleotides, more preferably 20 to 70 nucleotides, yet more preferably 20 to 50 nucleotides, and yet more preferably 20 to 30 nucleotides. In embodiments where is desired to optimise inducibility and power of the hypoxia-inducible promoter, the spacing between the final HBS and the TATA box (or equivalent sequence if a TATA box is not present) of the minimal promoter is preferably from 20 to 30 nucleotides. In embodiments where a somewhat lower level of inducibility and power is desired, the spacing between the final HBS and the TATA box (or equivalent sequence if a TATA box is not present) can be lesser or greater, e.g. from 0 to 10 nucleotides or from 31 to 100 nucleotides. While it is often desirable to maximise inducibility and power of the promoter, in some cases a lower level of inducibility and power may be desired.

In some specific preferred embodiments of the present invention, the hypoxia-inducible promoter suitably comprises one of the following sequences (the HBS sequences are underlined and minimal promoter sequences are shown in bold):
ACGTGCGATGATGCGTAGCTAGTAGTGATGATGCGTAGCTAGTAGTACGTGCGATG ATGCGTAGCTAGTAGTGATGATGCGTAGCTAGTAGTACGTGCGATGATGCGTAGCT AGTAGTGATGATGCGTAGCTAGTAGTACGTGCGATGATGCGTAGCTAGTAGTGATG ATGCGTAGCTAGTAGTACGTGCTTGGTACCATCCGGGCCGGCCGCTTAAGCGACG CCTATAAAAAATAGGTTGCATGCTAGGCCTAGCGCTGCCAGTCCATCTTCGCTAG CCTGTGCTGCGTCAGTCCAGCGCTGCGCTGCGTAACGGCCGCC (Synp-RTV-015; SEQ ID NO: 81), or a functional variant that comprises a sequence that is at least 80% identical thereto, preferably 85%, 90%, 95% or 99% identical thereto;
CTGCACGTAGATGATGCGTAGCTAGTAGTCTGCACGTAGATGATGCGTAGCTAGTA GTCTGCACGTAGATGATGCGTAGCTAGTAGTCTGCACGTAGATGATGCGTAGCTAG TAGTCTGCACGTAGATGATGCGTAGCTAGTAGTCTGCACGTAGTAGTCGTATGCTG ATGCGCAGTTAGCGTAGCTGAGGTACCGTCGACGATATCGGATCCAGGTCTATATA AGCAGAGCTCGTTTAGTGAACCGTCAGATCGCCTAGATACGCCATCCACGCTGTT TTGACCTCCATAGAAGATCGCCACC (Synp-RTV-016, SEQ ID NO: 82), or a functional variant that comprises a sequence that is at least 80% identical thereto, preferably 85%, 90%, 95% or 99% identical thereto;
CTGCACGTACTGCACGTACTGCACGTACTGCACGTATGGGTACCGTCGACGATATC GGATCCAGGTCTATATAAGCAGAGCTCGTTTAGTGAACCGTCAGATCGCCTAGAT ACGCCATCCACGCTGTTTTGACCTCCATAGAAGATCGCCACC (Synp-HYP-001, SEQ ID NO: 83) or a functional variant that comprises a sequence that is at least 80% identical thereto, preferably 85%, 90%, 95% or 99% identical thereto;
ACCTTGAGTACGTGCGTCTCTGCACGTATGGCGATTAAGACCTTGAGTACGTGCGT CTCTGCACGTATGGCGATTAAGACCTTGAGTACGTGCGTCTCTGCACGTATGGCGA TTAAGACCTTGAGTACGTGCGTCTCTGCACGTATGGCGATTAATCCATATGCTCTAG AGGGTATATAATGGGGGCCA (part of Synp-HYBT, SEQ ID NO: 84), or a functional variant that comprises a sequence that is at least 80% identical thereto, preferably 85%, 90%, 95% or 99% identical thereto; and
ACCTTGAGTACGTGCGTCTCTGCACGTATGGCGATTAAGACCTTGAGTACGTGCGT CTCTGCACGTATGGCGATTAAGACCTTGAGTACGTGCGTCTCTGCACGTATGGCGA TTAAGACCTTGAGTACGTGCGTCTCTGCACGTATGGCGATTAATCCATATGCAGGTC TATATAAGCAGAGCTCGTTTAGTGAACCGTCAGATCGCCTAGATACGCCATCCAC GCTGTTTTGACCTCCATAGAAGATCGCCACC (part of Synp-HV3C, SEQ ID NO: 85), or a functional variant that comprises a sequence that is at least 80% identical thereto, preferably 85%, 90%, 95% or 99% identical thereto.

Typically, it is preferred that in such functional variants the HRE1, HRE2, HRE3 and MP sequences are substantially identical to the reference sequence, and substantially all sequence variation arises in the spacer sequences.

In some preferred embodiments of the invention, upon induction via rendering cells hypoxic (e.g. after cells are exposed to 5% oxygen for 5 h, having previously been normoxic, e.g. exposed to 20% oxygen) the expression level of the transgene is increased by at least a 5-fold, more preferably a 10-, 15-, 20-, 30-, or 50-fold.

In some preferred embodiments of the invention, upon induction (e.g. after cells are exposed to 5% oxygen for 5 h, having previously been normoxic, e.g. exposed to 20% oxygen) the expression level of the transgene is at least 50% of that provided by the CMV-IE promoter (i.e. an otherwise identical vector in the same cells under the same conditions, but in which expression of the transgene is under control of CMV-IE rather then the hypoxia inducible promoter). More preferably the expression level of the transgene is at least 75%, 100%, 150%, 200%, 300%, 400% or 500% of that provided by the CMV-IE promoter.

The transgene typically encodes a product of interest, which may be a protein of interest or polypeptide of interest. The protein of interest or polypeptide of interest can be a protein, polypeptide, a peptide, a fusion protein, all of which can be expressed in a host cell and optionally secreted therefrom.

Suitable proteins and polypeptides of interest are presented hereinabove in relation to the fourth aspect of the present invention.

The product of interest may also be a nucleic acid, for example an RNA, for example an antisense RNA, microRNA, siRNA, tRNA, rRNAs, or any other regulatory, therapeutic or otherwise useful RNA.

The bioprocessing vector can be any naturally occurring or synthetically generated constructs suitable for uptake, proliferation, expression or transmission of nucleic acids in a cell, e.g. plasmids, minicircles, phagemids, cosmids, artificial chromosomes/mini-chromosomes, bacteriophages, viruses such as baculovirus, retrovirus, adenovirus, adeno-associated virus (AAV), herpes simplex virus, or bacteriophages. Methods for the construction of vectors are well known to the person skilled in the art, and they are described in various publications and reference texts. In particular, techniques for constructing suitable vectors, including a description of the functional and regulatory components such as promoters, enhancers, termination and polyadenylation signals, selection markers, origins of replication, and splicing signals, are known to the person skilled in the art. In preferred embodiments, the vector may be a eukaryotic expression vector. Eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operably linked, are well known in the art and several are commercially available from companies such as Stratagene, La Jolla, Calif.; Invitrogen, Carlsbad, Calif.; and Promega, Madison, Wis.

In some embodiments of the invention, the bioprocessing vector is an expression vector for expression in eukaryotic cells. Examples of eukaryotic expression vectors include, but are not limited to, pW-LNEO, pSV2CAT, pOG44, pXTI and pSG available from Stratagene; pSVK3, pBPV, pMSG and pSVL available from Amersham Pharmacia Biotech; and pCMVDsRed2-express, pIRES2-DsRed2, pDsRed2-Mito, pCMV-EGFP available from Clontech. Many other vectors are well-known and commercially available. For mammalian cells adenoviral vectors, the pSV and the pCMV series of vectors are particularly well-known non-limiting examples. There are many well-known yeast expression vectors including, without limitation, yeast integrative plasmids (YIp) and yeast replicative plasmids (YRp). For plants the Ti plasmid of Agrobacterium is an exemplary expression vector, and plant viruses also provide suitable expression vectors, e.g. tobacco mosaic virus (TMV), potato virus X, and cowpea mosaic virus.

In some embodiments of the invention, the vector is a plasmid. Such a plasmid may include a variety of other functional nucleic acid sequences, such as one or more selectable markers, one or more origins of replication, polycloning sites and the like.

In some embodiments of the invention the vector is episomal or it may be integrated into the genome of a cell.

In a seventh aspect there is provided a gene therapy vector comprising a CRE according to the first aspect of the present invention, CRM according to the second aspect of the present invention, a promoter according to the third aspect of the present invention or expression cassette according to the fourth aspect of the present invention.

It is generally preferred that the gene therapy vector is a viral vector, such as a retroviral, lentiviral, adenoviral, or adeno-associated viral (AAV) vector, but other forms of gene therapy vector are also contemplated. In some preferred embodiments the vector is an AAV vector. In some preferred embodiments the AAV has a serotype suitable for liver transduction. In some embodiments, the AAV is selected from the group consisting of: AAV2, AAV5, AAV6, AAV7, AAV8, AAV9, or derivatives thereof. AAV vectors are suitably used as self-complementary, double-stranded AAV vectors (scAAV) in order to overcome one of the limiting steps in AAV transduction (i.e. single-stranded to double-stranded AAV conversion), although the use of single-stranded AAV vectors (ssAAV) is also encompassed herein. In some embodiments of the invention, the AAV vector is chimeric, meaning it comprises components from at least two AAV serotypes, such as the ITRs of an AAV2 and the capsid protein of an AAV5.

In a eighth aspect there is provided a recombinant virion (viral particle) comprising a gene therapy vector according to the seventh aspect of the present invention. The virion can be produced using conventional techniques known to the skilled person.

In a ninth aspect there is provided a pharmaceutical composition comprising a gene therapy vector according to the seventh aspect of the present invention or virion of the eighth aspect of the present invention.

The pharmaceutical composition may be formulated with a pharmaceutically acceptable excipient, i.e., one or more pharmaceutically acceptable carrier substances and/or additives, e.g., buffers, carriers, excipients, stabilisers, etc. The pharmaceutical composition may be provided in the form of a kit. The term “pharmaceutically acceptable” as used herein is consistent with the art and means compatible with the other ingredients of the pharmaceutical composition and not deleterious to the recipient thereof.

In a tenth aspect there is provided a cell comprising a CRE according to the first aspect of the present invention, CRM according to the second aspect of the present invention, promoter according to the third aspect of the present invention, expression cassette according to the fourth aspect of the present invention or vector according to the fifth, sixth or seventh aspects of the invention. The cell may be present, for example, in cell culture or may be in vivo.

The CRE according to the first aspect, CRM according to the second aspect, promoter according to the third aspect, expression cassette according to the fourth aspect or vector according to the fifth, sixth or seventh aspect may be episomal or it may be integrated into the genome of the cell.

The cell may be present, for example, in cell culture or may be in vivo.

Suitable cells include, but are not limited to, eukaryotic cells, such as yeast, plant, insect or mammalian cells. For example, the cells may be any type of differentiated cells or may, be oocytes, embryonic stem cells, hematopoietic stem cells or other form. In some preferred embodiments the cell is an animal (metazoan) cell (e.g. a mammalian cell). In some preferred embodiments the cell is a mammalian cell. In some preferred embodiments the mammalian cell is a human, simian, murine, rat, rabbit, hamster, goat, bovine, sheep or pig cell. Particularly preferred cells or “host cells” for the production of products of interest are human, mice, rat, monkey, or rodent cell lines. Hamster cells are preferred in some embodiments, e.g. BHK21, BHK TK⁻, CHO, CHO-K1, CHO-DUKX, CHO-DUKX B1, CHO-S and CHO-DG44 cells, or derivatives/progenies of any of such cell lines. In alternative embodiments, the cell could be a human cell. In some preferred embodiments the human cell could be a human embryonic kidney (HEK) cell, preferably a HEK 293F cell. In another preferred embodiment of the invention the cell may be a retinal cell, e.g. a retinal pigmented epithelium (RPE) cell, for example ARPE-19 (ATCC CRL-2302). Furthermore, murine myeloma cells, preferably NS0 and Sp2/0 cells or the derivatives/progenies of any of such cell lines are also well-known as production cell lines for biopharmaceutical proteins. Non-limiting examples of cell lines that can be used in the present invention and sources from which they can be obtained are summarised in Table 1. Suitable host cells are commercially available, for example, from culture collections such as the DSMZ (Deutsche Sammlung von Mikroorganismen and Zeilkuituren GmbH, Braunschweig, Germany) or the American Type Culture Collection (ATCC).

TABLE 1
Source for cell lines suitable for use in the invention.

| Cell Line | Source |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHO-DUKX (also CHO duk⁻, | ATCC CRL-9096 |
| CHO/dhfr⁻) | |
| CHO-DUKX B11 | ATCC CRL-9010 |
| CHO-DG44 | Urtaub et al., Cell 33 (2), 405-412, 1983 |
| CHO Pro-5 | ATCC CRL-1781 |
| CHO-S | Invitrogen Cat No. 10743-029 |
| Led 3 | Stanley P. et al. Ann. Rev. Genetics |
| | 18, 525-552, 1984 |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNSI | ATCC CRL-8644 |
| Per.C6 | Fallaux, F. J. et al., Human Gene |
| | Therapy 9(13). 1909-1917, 199 |
| CHL | ECACC No. 87111906 |

Particularly preferred cells are human liver cells (especially Huh7 cells), human muscle cells (especially C2C12 cells), human embryonic kidney cells (especially, HEK-293 cells, and in particular HEK-293-F cells), CHO cells (particularly CHO-K1SV cells).

For bioprocessing, it may be preferred that cells are established, adapted, and completely cultivated under serum free conditions, and optionally in media which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif.), CHO-S-SFMII (Invtirogen), serum-free CHO Medium (Sigma), protein-free CHO Medium (Sigma), EX-CELL Media (SAFC), CDM4CHO and SFM4CHO (HyClone) are exemplary appropriate nutrient solutions. Any of the media may be supplemented as necessary with a variety of compounds examples of which are hormones and/or other growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics, trace elements. Any other necessary supplements may also be included at appropriate concentrations that would be known to those skilled in the art. In the present invention the use of serum-free medium is preferred, but media supplemented with a suitable amount of serum can also be used for the cultivation of host cells. For the growth and selection of genetically modified cells expressing a selectable gene a suitable selection agent is added to the culture medium.

The cell may be a prokaryotic cell, e.g. a bacterial cell. In some embodiments of the invention the cell may be a prokaryotic cell; although prokaryotic cells do not possess the inducible systems associated with the present invention, prokaryotic cells may nonetheless be useful in production of the bioprocessing vector or other steps in handling, transportation or storage of the bioprocessing vector.

In an eleventh aspect there is provided a cell culture comprising a population of cells according to the tenth aspect of the present invention and medium sufficient to support growth of the cells.

In a twelfth aspect there is provided a method for producing an expression product, the method comprising the steps of:

a) providing a population of eukaryotic cells comprising an expression cassette according to the fourth aspect of the present invention or a vector according to the fifth, sixth or seventh aspect of the present invention;
b) culturing said population of cells;
c) treating said population of cells so as to induce expression of the transgene present in the expression cassette or vector and thereby produce an expression product; and
d) recovering the expression product from said population of cells.

In some embodiments, there is provided a method for producing an expression product, the method comprising the steps of:
a) providing a population of eukaryotic cells comprising a synthetic expression cassette according to fourth aspect of the present invention;
b) culturing said population of cells;
c) treating said population of cells so as to induce expression of the transgene present in the expression cassette and thereby produce an expression product; and
d) recovering the expression product from said population of cells.

In some embodiments, the population of cells is treated so as to induce expression of the transgene by administering an inducer to the cells, suitably an inducer that activates adenylyl cyclase.

In some embodiments, the present invention provides a method for producing an expression product, the method comprising the steps of:

(a) providing a population of eukaryotic cells, preferably animal cells, most preferably mammalian cells comprising a bioprocessing vector according to the sixth aspect of the present invention;
(b) culturing said population of cells; and
(c) treating said population of cells so as to induce hypoxia in the cells, such that expression from the transgene linked to the hypoxia-inducible promoter is induced and the expression product is produced; and
(d) recovering the expression product.

The method is suitably a cell culture method. In some embodiments, the method is a method of bioprocessing, i.e. a process that uses living cells to obtain desired products. Preferred transgenes and products of interest that they encode are discussed above. The expression product may be useful for therapeutic, cosmetic, research or other industrial processes.

Step (b) typically comprises maintaining said population of cells under suitable conditions for proliferation of the cells. The conditions typically prepare the cells for expression of the expression product from the transgene upon induction in step (c). The cells may therefore be provided in suitable cell culture condition for the type of cell being used. Suitable cell culture conditions for the various cell types are well-known to the skilled person or can be readily identified from the literature.

The synthetic expression cassette can be present in the genome or can be episomal. The synthetic expression cassette can be stable or transient in the cell.

It will be apparent that the present invention allows for the production of the expression product to be delayed until a desired point in a cell culture process. This can, for example, permit the population of cells to be expanded until such time as a desired cell number or concentration is reached, or a desired growth phase is reached. This can be desirable for many reasons, e.g. to allow cells to grow under optimal conditions prior to expression of the transgene, which may inhibit growth. In the case of toxic proteins, for example, the production of a toxic expression product can be avoided until a cell culture system is at a desired stage. Once the toxic protein is expressed the cells will of course be adversely affected or killed. However, even for non-toxic expression products there may be considerable efficiency advantages in delaying expression of the transgene until a desired point.

The method suitably comprises incubating said population of cells under conditions suitable for growth of the cells prior to step (c) of treating said population of cells so as to induce expression of the transgene (e.g. by hypoxia or by inducer that activates adenylyl cyclase).

In some embodiments, the population of cells is treated so as to induce expression of the transgene by treating the cells in any means which leads to them becoming hypoxic.

Suitably, step (c) comprises treating the cells in any means which leads to them becoming hypoxic. Suitable approaches will be apparent to the skilled person for any particular cell type. Generally, eukaryotic cells are cultured under aerobic conditions, and many approaches are known in the art to achieve this for various cell and culture types. Hypoxic conditions can be achieved by reducing the amount of oxygen supplied to the cell. For example, cells can be grown under normoxic conditions (e.g. approximately 20% oxygen), before being switched to a gas mix comprising less oxygen or no oxygen to induce hypoxia. For example, a gas containing 5% oxygen can be used to induce hypoxia in cells. An exemplary suitable gas mix for use to induce hypoxic conditions in cell culture is 5% oxygen, 10% carbon dioxide and 85% nitrogen, but other gas mixes can be used.

In an alternative approach, hypoxia in cell culture can be induced by introduction of an agent that can induce hypoxia in the cells. For example, CoCl₂ can be used at suitable concentrations, e.g. a final concentration of approximately 100 pM in cell culture media, to induce hypoxia. Generally, it is preferred in the present invention that hypoxia is achieved without the addition of such an agent as it adds to costs, and in many cases the agent will be undesirable and may be hard to remove.

In some cases, it may be desirable to alter the amount of oxygen supplied to the cells while they are in a hypoxic condition to optimize or otherwise modulate expression of the desired expression product. For example, it may be desirable to establish highly hypoxic conditions initially to strongly induce hypoxic conditions, followed by a period of culturing the cells under less hypoxic conditions that are less detrimental to the health and activity of the cells. Thus step (c) may comprise varying the level of hypoxia to which the cells are subject.

In some embodiments, the population of cells is treated so as to induce expression of the transgene by treating the cells in any means which leads to an activation of adenylyl cyclase.

Step (c) preferably comprises activating adenylate cyclase in the cells, leading to elevation of levels of intracellular cAMP.

Step (c) typically comprises administering an inducer to the cells. The inducer can be any agent that activates adenylyl cyclase. It is of course preferred that the inducer is not significantly toxic or otherwise deleterious to the cells.

Suitable inducers that are able to activate adenylyl cyclase include, but are not limited to:
Forskolin (a potent adenylyl cyclase activator (CAS Number 66575-29-9));
NKH 477 (a water-soluble analogue of forskolin (CAS Number 138605-00));
PACAP-27 (a neuropeptide that stimulates adenylate cyclase (CAS Number 127317-03-7));
PACAP-38 (a neuropeptide that stimulates adenylate cyclase (CAS Number 137061-48-4));
Pertussis toxin CAS Number 70323-44-3; and
Cholera toxin (CAS Number 9012-63-9).

All of the above are commercially available from Sigma-Aldrich, Inc (now part of Merck KGaA).

In particularly preferred embodiments of the invention the inducer comprises forskolin or NKH 477.

Forskolin is classified as generally regarded as safe (GRAS), which is generally desirable from a safety point of view. Forskolin (also known as coleonol) is a labdane diterpene that is produced by the Indian Coleus plant (Plectranthus barbatus). Forskolin is a commonly used in material research to increase levels of cyclic AMP. Forskolin is also used in traditional medicine. Since forskolin is GRAS, it is a preferred inducer for the promoters according to this invention in gene therapy applications.

NKH 477 is a water-soluble analogue of forskolin and therefore may be advantageous in terms of ease of use in cell culture in particular. Since NKH477 is water-soluble, it is a preferred inducer for the promoters according to this invention in bioprocessing applications.

The inducer can be administered to the cells in any suitable manner. For example, the inducer can be added to the culture medium, if necessary with a suitable carrier, surfactant or suchlike.

A suitable dosage rate for any given inducer can be readily determined by the person skilled in the art. The person skilled in the art can thus readily determine for any inducer an appropriate way to deliver the inducer to the cells, and a suitable concentration to use. In general terms, the inducer may be administered at any suitable concentration in the range of from 1 nM to 1000 pM, optionally in the range of from 0.1 pM to 100 pM.

Forskolin may suitably be administered to the cells at a concentration of from 0.1 pM to 1000 pM, more preferable from 1 pM to 100 pM, yet more preferably from 5 pM to 30 pM. For example, administration of a concentration of about 18 pM to cells was demonstrated in the examples below to be optimum to induce expression from the RTV-019 construct in CHO-K1SV cells.

NKH 477 may suitably be administered to the cells at a concentration of from 0.1 pM to 1000 pM, more preferable from 1 pM to 100 pM, yet more preferably from 2 pM to 20 pM. For example, administration of a concentration of about 8 pM to cells was demonstrated in the examples below to be optimum to induce expression from the RTV-019 construct in CHO-K1SV cells.

The method may suitably comprise ceasing to administer the inducer. Ceasing to administer the inducer will lead to at least a reduction of expression of the expression product. Typically, expression of the expression product will return to a baseline level over time.

The method may suitably comprise varying the concentration of the inducer administered to the cells over time. This can be used to modulate the level of expression of the expression product.

In some embodiments the method may comprise administering to the cells an inhibitor of adenylyl cyclase, which acts to reduce or turn off expression of the expression product. Inhibitors of adenylyl cyclase include, but are not limited to:
NB001—inhibitor of adenylyl cyclase 1 (AC1)
9-Cyclopentyladenine monomethanesulfonate—stable, cell-permeable, non-competitive adenylyl cyclase inhibitor
SQ 22,536—cell-permeable adenylyl cyclase inhibitor
MDL-12,330A hydrochloride—adenylyl cyclase inhibitor
2′,5′-Dideoxyadenosine—cell-permeable adenylyl cyclase inhibitor
2′,5′-Dideoxyadenosine 3′-triphosphate tetrasodium salt—potent inhibitor of adenylyl cyclase
MANT-GTPγS—potent and competitive adenylyl cyclase inhibitor
2′,3′-Dideoxyadenosine—specific adenylyl cyclase inhibitor
NKY80—selective adenylyl cyclase-V inhibitor
KH7—selective inhibitor of soluble adenylyl cyclase

All of the above are commercially available from Sigma-Aldrich, Inc (now part of Merck KGaA).

The population of eukaryotic cells can be any type of cell suitable for cell culture. In some preferred embodiments, the population of eukaryotic cells is a population of mammalian cells. There is a wide range of mammalian cells that can be used, many of which are discussed above. Preferred mammalian cells include, without limitation, chinese hamster ovary (CHO), human liver cells, human muscle cells, human embryonic kidney (HEK) cells, human embryonic retinal cells, human amniocyte cells, and Mouse myeloma lymphoblastoid cells. Particularly preferred cells are human liver cells (especially Huh7 cells), human muscle cells (especially C2C12 cells), human embryonic kidney cells (especially, HEK-293 cells, and in particular HEK-293-F cells), CHO cells (particularly CHO-K1SV cells).

Step (d), i.e. recovering the expression product from said population of cells, can be carried out using conventional techniques well-known in the art. It typically comprises separating the expression product from said population of cells, and in some cases from other components of the cell culture medium. The method preferably comprises the step of purifying the expression product. Suitable methods of recovering and/or purifying an expression product are conventional in the art, and the methods chosen will depend on the specific nature of the expression product.

In some embodiments, the method may suitably comprise the step of introducing the expression cassette into the cells. There are many well-known methods of transfecting eukaryotic cells, and the skilled person could readily select a suitable method for any cell type. The expression cassette can of course be provided in any suitable vector, as discussed above. In some embodiments, the method comprises the step of introducing into the cell a bioprocessing vector as described herein. Methods for introducing a vector into the various cells suitable for use in the present invention are well known in the art.

The methods can be carried out in any suitable reactor including but not limited to stirred tank, airlift, fibre, microfibre, hollow fibre, ceramic matrix, fluidized bed, fixed bed, and/or spouted bed bioreactors. As used herein, “reactor” can include a fermenter or fermentation unit, or any other reaction vessel and the term “reactor” is used interchangeably with “fermenter”. For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing. Example reactor units, such as a fermentation unit, may contain multiple reactors within the unit, for example the unit can have 1 to 10 or more bioreactors in each unit. In various embodiments, the bioreactor can be suitable for batch, semi fed-batch, fed-batch, perfusion, and/or a continuous fermentation process. In some embodiments, the bioreactor can have a volume of from about 100 ml to about 50,000 litres, preferably 10 litres or higher. Additionally, suitable reactors can be multi-use, single-use, disposable, or non-disposable and can be formed of any suitable material. U.S. Publication Nos. 2013/0280797, 2012/0077429, 2011/0280797, 2009/0305626, and U.S. Pat. Nos. 8,298,054, 7,629,167, and 5,656,491 (hereby incorporated by reference in their entirety) describe exemplary systems that may be used in the present invention.

In a thirteenth aspect, the invention provides a reactor vessel comprising a cell culture comprising cells according to the tenth aspect of the invention and a medium sufficient to support growth of the cell.

Various reactors suitable for the present invention are described above. In embodiments, wherein the cells are induced by hypoxia, the reactor is preferably configured to allow both normoxic and hypoxic conditions to be applied to the cell culture, e.g. by controlling the amount of oxygen gas provided to the cell culture.

In a fourteenth aspect, the invention provides the use of vector according to the fifth, sixth or seventh aspect of the present invention or a cell according to the tenth aspect of the present invention in a bioprocessing method for the manufacture of a product of interest, e.g. a therapeutic product. Suitable methods are discussed above.

In a fifteenth aspect the present invention provides a method of gene therapy of a subject, preferably a human, in need thereof, the method comprising:
introducing into the subject a pharmaceutical composition comprising a gene therapy vector according to the seventh aspect of the present invention, the gene therapy vector comprising a sequence encoding a therapeutic expression product, such that the gene therapy vector delivers the nucleic acid expression construct to target cells of the subject; and
administering an inducer to the subject such that a therapeutically effective amount of the therapeutic expression product is expressed in the subject.

Gene therapy protocols have been extensively described in the art. These include, but are not limited to, intramuscular injection of a suitable vector, hydrodynamic gene delivery in various tissues, including muscle, interstitial injection, instillation in airways, application to endothelium, intra-hepatic parenchyme, and intravenous or intra-arterial administration. Various devices have been developed for enhancing the availability of DNA to the target cell. A simple approach is to contact the target cell physically with catheters or implantable materials containing DNA. Another approach is to utilize needle-free, jet injection devices which project a column of liquid directly into the target tissue under high pressure. These delivery paradigms can also be used to deliver vectors. Another approach to targeted gene delivery is the use of molecular conjugates, which consist of protein or synthetic ligands to which a nucleic acid- or DNA-binding agent has been attached for the specific targeting of nucleic acids to cells (Cristiano et al., 1993).

Expression levels of the expression product (e.g. protein) can be measured by various conventional means, such as by antibody-based assays, e.g. a Western Blot or an ELISA assay, for instance to evaluate whether therapeutic expression of the expression product is achieved. Expression of the expression product may also be measured in a bioassay that detects an enzymatic or biological activity of the gene product.

The therapeutic product may have a therapeutic effect in any suitable location in the subject. For example, it may have an effect in the cells where it is expressed, in neighbouring cells or tissues, or it may be secreted and enter the bloodstream and treat a condition elsewhere in the body.

Various inducers that can be used in the present invention are discussed above. Forskolin is a particularly preferred inducer as it is GRAS and can be safely administered to humans. However, other pharmaceutically acceptable inducers can be used.

The inducer can be delivered directly to a target suite (e.g. by injection) or given systemically. A suitable dosage rate for any given inducer can be readily determined by the person skilled in the art. The person skilled in the art can thus readily determine for any inducer an appropriate way to deliver the inducer to the cells, and a suitable concentration to use.

The method may suitably comprise ceasing to administer the inducer to the subject. Ceasing to administer the inducer will lead to at least a reduction of expression of the expression product. Typically, expression of the expression product will return to a baseline level over time. Administration of the inducer may, for example be ceased after a suitable therapeutic benefit has been achieved.

The method may suitably comprise varying the amount of the inducer administered to the subject over time. This can be used to modulate the level of expression of the therapeutic product provided in a subject. The amount of the inducer administered to the subject may be adjusted in order to obtain expression of a desired amount (dose) of the therapeutic product. Thus, where there is a clinical need for an increased amount of the therapeutic product (e.g. due to insufficient response in the subject), the amount of the inducer administered to the subject can be increased, and vice versa (e.g. due to an excessive response or undesirable side effects). The amount can be varied in response to an alteration in the condition of a subject, the level of a biomarker in a subject, or any other reason. Thus, in some preferred embodiments of the invention the concentration of the inducer administered to the subject over time is varied in order to modulate the dosage of a therapeutic product provided in the subject.

In some embodiments the method may comprise the steps of:
determining the amount of the therapeutic product expressed in the subject or assessing the response of a subject to the therapeutic product, and:
a) where a higher amount of the therapeutic product in the subject is desired, increasing the amount of inducer administered to the subject, or
b) where a lower amount of the therapeutic product in the subject is desired, decreasing the amount of inducer administered to the subject.

When considering that the amount of the inducer administered to the subject may be varied over time, it will of course be understood that the inducer will not typically be administered to the patient continuously, but rather will typically be administered at a given dosage level at a given time interval. The present invention thus contemplates varying the amount of inducer administered to the subject over time by adjusting the dose, adjusting the time period between doses, or both. Thus, for example, to increase the amount of inducer administered to a subject the dose can be increased while the time period between doses is kept constant, the dose can be kept constant while the time period between doses is reduced, or the dose can be increased and the time period between doses is reduced. To decrease the amount of inducer administered to a subject the dose can be decreased while the time period between doses is kept constant, the dose can be kept constant while the time period between doses is reduced, or the dose can be decreased and the time period between doses is increased.

Alternatively, or additionally, the method may comprise changing the inducer in order to alter the amount of the therapeutic product in the subject. For example, a weak inducer can be replaced with a stronger inducer, or vice versa.

The method may also comprise changing the inducer if, for example, the subject has an adverse reaction to an inducer, or the inducer is found to be ineffective in the subject.

In some embodiments the method may comprise administering to the cells an inhibitor of adenylyl cyclase, which acts to reduce or turn off expression of the expression product.

Inhibitors of adenylyl cyclase are discussed above. As for the inducer, the inhibitor of adenylyl cyclase should be pharmaceutically acceptable.

Genes encoding suitable therapeutic gene products are discussed above.

Suitably the gene therapy vector is a viral gene therapy vector, preferably an AAV vector.

In some embodiments, the method comprises administering the gene therapy vector systemically. Systemic administration may be enteral (e.g. oral, sublingual, and rectal) or parenteral (e.g. injection). Preferred routes of injection include intravenous, intramuscular, subcutaneous, intra-arterial, intra-articular, intrathecal, and intradermal injections.

In some embodiments, the gene therapy vector may be administered concurrently or sequentially with one or more additional therapeutic agents or with one or more saturating agents designed to prevent clearance of the vectors by the reticular endothelial system.

Where the gene therapy vector is an AAV vector, the dosage of the vector may be from 1×10¹⁰ gc/kg to 1×10¹⁵ gc/kg or more, suitably from 1×10¹² gc/kg to 1×10¹⁴ gc/kg, suitably from 5×10¹² gc/kg to 5×10¹³ gc/kg.

In general, the subject in need thereof will be a mammal, and preferably primate, more preferably a human. Typically, the subject in need thereof will display symptoms characteristic of a disease. The method typically comprises ameliorating the symptoms displayed by the subject in need thereof, by expressing the therapeutic amount of the therapeutic product.

In a sixteenth aspect, the invention provides the expression cassettes according to the fourth aspect of the present invention, vectors according to the fifth, sixth or seventh aspect of the present invention, virions according to the eight aspect of the present invention, cells according to the tenth aspect of the present invention or pharmaceutical compositions according to the ninth aspect of the present invention for use in a method of treatment or therapy. Suitable methods of therapy are discussed above.

In a seventeenth aspect there is provided an expression cassette according to the fourth aspect of the present invention, vector according to the fifth, sixth or seventh aspect of the present invention, virion according to the eighth aspect of the present invention or cells according to the tenth aspect of the present invention for use in the manufacture of a pharmaceutical composition, optionally for use in a bioprocessing method for the manufacture of a product of interest, e.g. a therapeutic product.

In a eighteenth aspect of the present invention there is provided a synthetic HRE comprising one of the following sequences:
a) [ACGTGC-S]n-ACGTGC (SEQ ID NO: 64); wherein S is a spacer and n is from 2 to 9, preferably from 3 to 7;
b) [CTGCACGTA-S]ₙ-CTGCACGTA (SEQ ID NO: 69); wherein S is a spacer and n is from 2 to 9, preferably from 3 to 7; and
c) [ACCTTGAGTACGTGCGTCTCTGCACGTATG-S]ₙ-ACCTTGAGTACGTGCGTCTCTGCACGTATG (SEQ ID NO: 75); wherein S is a spacer and n is from 2 to 5, preferably from 2 to 4, preferably 3.

Various optional and preferred features of the HREs are discussed in detail in respect of the sixth aspect of the invention, and these of course apply equally to this aspect of the invention (for brevity they will not be repeated). In particular, preferred lengths for the spacer are set out above.

In some preferred embodiments of the eighteenth aspect of the invention, there is provided a synthetic HRE comprising or consisting of one of the following sequences:
a) ACGTGCGATGATGCGTAGCTAGTAGTGATGATGCGTAGCTAGTAGTACGTGCGATG ATGCGTAGCTAGTAGTGATGATGCGTAGCTAGTAGTACGTGCGATGATGCGTAGCT AGTAGTGATGATGCGTAGCTAGTAGTACGTGCGATGATGCGTAGCTAGTAGTGATG ATGCGTAGCTAGTAGTACGTGC (SEQ ID NO: 68), or a functional variant that is that is at least 80% identical thereto, preferably 85%, 90%, 95% or 99% identical thereto;
b) CTGCACGTAGATGATGCGTAGCTAGTAGTCTGCACGTAGATGATGCGTAGCTAGTA GTCTGCACGTAGATGATGCGTAGCTAGTAGTCTGCACGTAGATGATGCGTAGCTAG TAGTCTGCACGTAGATGATGCGTAGCTAGTAGTCTGCACGTA (SEQ ID NO: 73), or a functional variant that comprises a sequence that is at least 80% identical thereto, preferably 85%, 90%, 95% or 99% identical thereto; or
c) ACCTTGAGTACGTGCGTCTCTGCACGTATGGCGATTAAGACCTTGAGTACGTGCGT CTCTGCACGTATGGCGATTAAGACCTTGAGTACGTGCGTCTCTGCACGTATGGCGA TTAAGACCTTGAGTACGTGCGTCTCTGCACGTATG (SEQ ID NO: 79), or a functional variant that comprises a sequence that is at least 80% identical thereto, preferably 85%, 90%, 95% or 99% identical thereto.

Typically, it is preferred that in such functional variants the HRE1 and HRE2 sequences are substantially identical, and substantially all variation arises in the spacer sequences.

In a nineteenth aspect of the present invention there is provided a hypoxia-inducible promoter comprising at least one HRE of the eighteenth aspect of the invention operably coupled to a minimal or proximal promoter, preferably a minimal promoter.

Various optional and preferred features of the hypoxia-inducible promoters (e.g. preferred minimal promoters and spacing between the HRE and promoter) are discussed in detail in respect of the sixth aspect of the invention, and these of course apply equally to this aspect of the invention (for brevity they will not be repeated).

In some specific preferred embodiments of the present invention, the hypoxia-inducible promoter suitably comprises one of the following sequences (the HBS sequences are underlined and minimal promoter sequences are shown in bold):
ACGTGCGATGATGCGTAGCTAGTAGTGATGATGCGTAGCTAGTAGTACGTGCGATG ATGCGTAGCTAGTAGTGATGATGCGTAGCTAGTAGTACGTGCGATGATGCGTAGCT AGTAGTGATGATGCGTAGCTAGTAGTACGTGCGATGATGCGTAGCTAGTAGTGATG ATGCGTAGCTAGTAGTACGTGCTTGGTACCATCCGGGCCGGCCGCTTAAGCGACG CCTATAAAAAATAGGTTGCATGCTAGGCCTAGCGCTGCCAGTCCATCTTCGCTAG CCTGTGCTGCGTCAGTCCAGCGCTGCGCTGCGTAACGGCCGCC (Synp-RTV-015; SEQ ID NO: 81), or a functional variant that comprises a sequence that is at least 80% identical thereto, preferably 85%, 90%, 95% or 99% identical thereto;
CTGCACGTAGATGATGCGTAGCTAGTAGTCTGCACGTAGATGATGCGTAGCTAGTA GTCTGCACGTAGATGATGCGTAGCTAGTAGTCTGCACGTAGATGATGCGTAGCTAG TAGTCTGCACGTAGATGATGCGTAGCTAGTAGTCTGCACGTAGTAGTCGTATGCTG ATGCGCAGTTAGCGTAGCTGAGGTACCGTCGACGATATCGGATCCAGGTCTATATA AGCAGAGCTCGTTTAGTGAACCGTCAGATCGCCTAGATACGCCATCCACGCTGTT TTGACCTCCATAGAAGATCGCCACC (Synp-RTV-016, SEQ ID NO: 82), or a functional variant that comprises a sequence that is at least 80% identical thereto, preferably 85%, 90%, 95% or 99% identical thereto;
ACCTTGAGTACGTGCGTCTCTGCACGTATGGCGATTAAGACCTTGAGTACGTGCGT CTCTGCACGTATGGCGATTAAGACCTTGAGTACGTGCGTCTCTGCACGTATGGCGA TTAAGACCTTGAGTACGTGCGTCTCTGCACGTATGGCGATTAATCCATATGCTCTAG AGGGTATATAATGGGGGCCA (part of Synp-HYBT, SEQ ID NO: 84), or a functional variant that comprises a sequence that is at least 80% identical thereto, preferably 85%, 90%, 95% or 99% identical thereto; and
ACCTTGAGTACGTGCGTCTCTGCACGTATGGCGATTAAGACCTTGAGTACGTGCGT CTCTGCACGTATGGCGATTAAGACCTTGAGTACGTGCGTCTCTGCACGTATGGCGA TTAAGACCTTGAGTACGTGCGTCTCTGCACGTATGGCGATTAATCCATATGCAGGTC TATATAAGCAGAGCTCGTTTAGTGAACCGTCAGATCGCCTAGATACGCCATCCAC GCTGTTTTGACCTCCATAGAAGATCGCCACC (part of Synp-HV3C, SEQ ID NO: 85), or a functional variant that comprises a sequence that is at least 80% identical thereto, preferably 85%, 90%, 95% or 99% identical thereto.

Typically, it is preferred that in such functional variants the HRE1, HRE2 and MP sequences are substantially identical, and substantially all variation arises in the spacer sequences.

In twentieth aspect of the present invention there is provided a gene therapy vector comprising an HRE or hypoxia-inducible promoter according to the eighteenth or nineteenth aspect of the invention operably linked to a transgene encoding a therapeutic expression product. Suitable therapeutic products are discussed above.

In some embodiments of the invention, the transgene can be useful for gene editing, e.g. a gene encoding a site-specific nuclease, such as a meganuclease, zinc finger nuclease

(ZFN), transcription activator-like effector-based nuclease (TALEN), or the clustered regularly interspaced short palindromic repeats system (CRISPR-Cas). Suitably the site-specific nuclease is adapted to edit a desired target genomic locus by making a cut (typically a site-specific double-strand break) which is then repaired via non-homologous end-joining (NHEJ) or homology dependent repair (HDR), resulting in a desired edit. The edit can be the partial or complete repair of a gene that is dysfunctional, or the knock-down or knock-out of a functional gene.

In some preferred embodiments of the invention, the gene therapy vector is a viral vector, such as a retroviral, lentiviral, adenoviral, or adeno-associated viral (AAV) vector, but other forms of gene therapy vector are also contemplated. In some embodiments the vector is an AAV vector. In some embodiments, the AAV is selected from the group consisting of: AAV2, AAV5, AAV6, AAV7, AAV8, AAV9, or derivatives thereof. AAV vectors are suitably used as self-complementary, double-stranded AAV vectors (scAAV) in order to overcome one of the limiting steps in AAV transduction (i.e. single-stranded to double-stranded AAV conversion), although the use of single-stranded AAV vectors (ssAAV) is also encompassed herein. In some embodiments of the invention, the AAV vector is chimeric, meaning it comprises components from at least two AAV serotypes, such as the ITRs of an AAV2 and the capsid protein of an AAV5.

In a twenty-first aspect the present invention provides a recombinant virion (viral particle) comprising a gene therapy vector according to the twentieth aspect of the present invention.

The gene therapy vectors or virions of the present invention may be formulated in a pharmaceutical composition with a pharmaceutically acceptable excipient, i.e., one or more pharmaceutically acceptable carrier substances and/or additives, e.g., buffers, carriers, excipients, stabilisers, etc. The pharmaceutical composition may be provided in the form of a kit.

Accordingly, in a twenty-second aspect the present invention provides a pharmaceutical composition comprising a gene therapy vector according to the twentieth aspect of the present invention or virion according to the twenty-first aspect of the present invention.

### BRIEF DESCRIPTION OF THE FIGURES

FIG. 1 is an illustration of the mechanism of action of forskolin and other adenylyl cyclase activators.

FIG. 2 shows the luciferase expression from the promoters RTV-017 and RTV-019 in Huh7 (liver) without induction and after induction with 20 pM forskolin. RTV-017 and RTV-019 promoters show low background activity without induction and high activity following induction. The activity of the CMV-IE promoter does not change.

FIG. 3 shows luciferase expression from the promoter RTV-019 in C2C12 (muscle) cells without induction and after induction with 20 pM forskolin. RTV-019 promoter shows low background activity without induction and high activity following induction. CMV-IE expression was not analysed under induced conditions, and thus no result is shown in the graph.

FIG. 4 shows a graph illustrating that when the promoters RTV-017, RTV-019 and FORCYB1 are expressed from an AAV vector (therefore containing ITRs) they still maintain low background activity and high levels of expression and induction in Huh7 cells. Luciferase activity was measured without induction and after induction with 20 pM forskolin or 7.2 pM NKH477. Similarly to FIGS. 2 to 4, RTV-017, FORCYB1 and RTV-019 show low background activity without induction and high activity following induction. CMV-IE shows weaker expression in AAV vector than the tested promoters.

FIG. 5 shows the activity of the promoters after transient transfection into the suspension cell line HEK293-F. The cells were induced (at time 0 h) with 20 pM forskolin and luciferase expression was measured at 0 h, 3 h, 5 h and 24 h. All constructs showed increase in activity (to a varying degree) while the activity of CMV-IE remained constant.

FIG. 6 shows the activity of the promoters after transient transfection into the suspension cell line CHO-K1SV with and without induction by 20 pM forskolin. Luciferase expression was measured 24 h after induction. All constructs showed increase in activity (to a varying degree) following induction.

FIG. 7 shows the response of the promoter RTV-019 to increasing concentrations of forskolin and NKH477 in the stably transfected cell line CHO-K1SV.

FIG. 8 shows the SEAP expression from the promoters in the stably transfected cell line CHO-K1SV with and without induction by 20 pM forskolin and 7.2 pM NKH477.

FIG. 9 shows the same data as in FIG. 8 but the activity is expressed compared to CMV-IE.

FIG. 10 shows brightfield microscopy pictures of C2C12 cells at passage 11. A) shows the cells prior to transformation (day 2). B) shows the C2C12 cells 24 hours after transfection (day 3). C) shows the differentiated C2C12 cells after 5.5 days into differentiation medium (day 7.5). Scale bar is 50 μm

FIG. 11A shows a schematic diagram of hypoxia-inducible gene expression. Transcription factor HIF1A (HIF1a) is degraded under normal oxygen conditions, but under hypoxic conditions, it is stabilised, dimerises with HIF1B (HIF1β) to form HIF1 and is translocated to the nucleus. In the nucleus, the HIF1 complex can bind to the hypoxia response element and initiate expression of the gene of interest.

FIG. 11B shows a schematic diagram of the structural organisation of HIF1α and HIF1β. Both HIF1α and HIF1β have a bHLH domain for DNA binding. HIF1β has a Per-ARNT-Sim (PAS) domain for central heterodimerisation and HIF1α's C terminal domain (TAD N/TAD C) recruits transcriptional coregulatory proteins. When HIF1α and HIF1β dimerise, they translocate to the nucleus and turn on expression of hypoxia-regulated genes after binding to a hypoxia-responsive element.

FIG. 12 shows a schematic diagram of promoters RTV-015, RTV-016, HV3C, HYB and Synp-HYP-001. RTV-015 promoter comprises of five HRE1 and a synthetic minimal promoter MP1. These elements are spaced apart with spacers (not shown). RTV-016 promoter comprises of 6 HRE2 and CMV minimal promoter. These elements are spaced apart with spacers (not shown). HV3C promoter comprises of 4 HRE3 and CMV minimal promoter. These elements are spaced apart with spacers (not shown). HYBT promoter comprises of 4 HRE3 and a YB-TATA minimal promoter. These elements are spaced apart with spacers (not shown). Synp-HYP-001 comprises of four HRE2 and a CMV minimal promoter. The HRE2 elements are not spaced apart with spacers but there is a spacer between the last HRE2 element and the CMV minimal promoter (not shown).

FIG. 13 shows a time course of luciferase expression from the RTV-015, HYBT, RTV-016, SYNP-HYP-011, CMV-IE and HV3C constructs in transiently transduced HEK293-F cells under hypoxia. Cells were placed in hypoxia at 0 hours and then luciferase activity monitored. Luciferase expression from the CMV minimal promoter, which was used as a control, does not change but the rest of the constructs show increase in luciferase activity with time.

FIG. 14 shows measurement of luciferase expression from the RTV-015, RTV-016, HV3C, HYBT and CMV-IE constructs in transiently transduced HEK293-T in normoxic conditions and after 24 hours in hypoxia. The luciferase expression from the CMV-IE promoter is the same in normoxia and hypoxia. RTV-015, RTV-016, HV3C, HYBT constructs show almost no luciferase activity in normoxia but are induced to a varying level after 24 hours in hypoxia with RTV-015 showing the lowest and HV3C showing the highest inducibility.

FIG. 15 shows measurement of luciferase expression from the RTV-015, RTV-016, HV3C, HYBT and CMV-IE constructs in transiently transduced CHO_GS suspension cell line in normoxic conditions and after 24 hours in hypoxia. The luciferase expression from the CMV-IE is the same in normoxia and hypoxia. Similar to the results shown in FIG. 4, RTV-015, RTV-016, HV3C, HYBT constructs show almost no luciferase activity in normoxia but are induced to a varying level after 24 hours in hypoxia with RTV-015 showing the lowest and HV3C showing the highest inducibility.

FIG. 16 shows measurement of SEAP expression from the RTV-015, RTV-016, HV3C, HYBT and CMV-IE constructs in stably integrated CHO-GSK1SV cell line in normoxia (24 hours after seeding—show as 0 h), followed by 24 h in normoxia or by 24 h in hypoxia. The SEAP expression from the CMV-IE construct is the same in normoxia and hypoxia. Similar to the results shown in FIGS. 4 and 5, RTV-015, RTV-016, HV3C, HYBT constructs show almost no SEAP activity in normoxia but are induced to a varying level after 24 hours in hypoxia. RTV-015 still shows the lowest inducibility and HV3C and HYBT, the highest.

FIG. 17 show cell numbers of the stably integrated CHO-GSK1SV with RTV-015, RTV-016, HV3C, HYBT and CMV-IE constructs. SEAP expression was normalised to the number of cells in the respective condition.

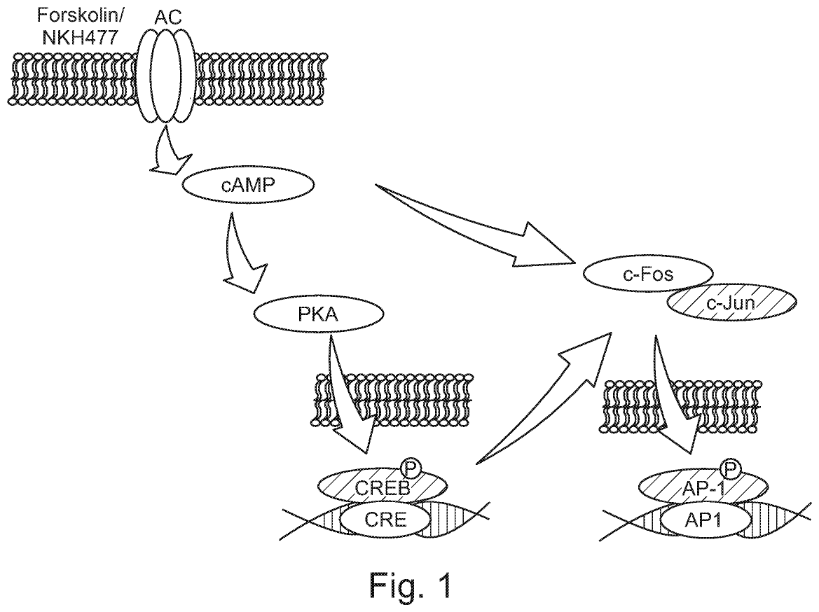

### DETAILED DESCRIPTION OF EMBODIMENTS OF THE INVENTION AND EXAMPLES

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds.-in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The terms “comprising”, “comprises” and “comprised of” as used herein are synonymous with “including”, “includes” or “containing”, “contains”, and are inclusive or open-ended and do not exclude additional, non-recited features, elements or method steps.

The recitation of numerical ranges by endpoints includes all numbers and fractions subsumed within the respective ranges, as well as the recited endpoints.

The term “cis-regulatory element” or “CRE”, is a term well-known to the skilled person, and means a nucleic acid sequence such as an enhancer, promoter, insulator, or silencer, that can regulate or modulate the transcription of a neighbouring gene (i.e. in cis). CREs are found in the vicinity of the genes that they regulate. CREs typically regulate gene transcription by binding to TFs, i.e. they include TFBS. A single TF may bind to many CREs, and hence control the expression of many genes (pleiotropy). CREs are usually, but not always, located upstream of the transcription start site (TSS) of the gene that they regulate. “Enhancers” are CREs that enhance (i.e. upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene. The term “silencer” can also refer to a region in the 3′ untranslated region of messenger RNA, that bind proteins which suppress translation of that mRNA molecule, but this usage is distinct from its use in describing a CRE. Generally, in the present invention, the CREs are forskolin inducible enhancers or hypoxia inducible enhancers. In the present context, it is preferred that the CRE is located 1500 nucleotides or less from the transcription start site (TSS), more preferably 1000 nucleotides or less from the TSS, more preferably 500 nucleotides or less from the TSS, and suitably 250, 200, 150, or 100 nucleotides or less from the TSS. CREs of the present invention are preferably comparatively short in length, preferably 50 nucleotides or less in length, for example they may be 40, 30, 20, 10 or 5 nucleotides or less in length.

The term “cis-regulatory module” or “CRM” means a functional module made up of two or more CREs; in the present invention the CREs are typically forskolin inducible enhancers or hypoxia inducible enhancers. Thus, in the present application a CRM typically comprises a plurality of forskolin inducible CREs or hypoxia inducible CREs. Typically, the multiple CREs within the CRM act together (e.g. additively or synergistically) to enhance the transcription of a gene that the CRM is operably associated with. There is conservable scope to shuffle (i.e. reorder), invert (i.e. reverse orientation), and alter spacing in CREs within a CRM. Accordingly, functional variants of CRMs of the present invention include variants of the referenced CRMs wherein CREs within them have been shuffled and/or inverted, and/or the spacing between CREs has been altered.

A “functional variant” of a cis-regulatory element, cis-regulatory module, promoter or other nucleic acid sequence in the context of the present invention is a variant of a reference sequence that retains the ability to function in the same way as the reference sequence, e.g. as a forskolin-inducible element or promoter or hypoxia-inducible element or promoter. Alternative terms for such functional variants include “biological equivalents” or “equivalents”.

A CRE can be considered “forskolin-inducible” if, when placed in a suitable promoter (as discussed in more detail herein), expression of a gene operably linked to said promoter can be induced by administration of forskolin to a eukaryotic cell (preferably a mammalian cell) containing said promoter.

It will be appreciated that the ability of a given CRE to function as a forskolin-inducible enhancer is determined principally by the ability of the sequence to be bound by CREB and/or AP1 (following induction by an activator of adenylyl cyclase and resulting increase in cellular cAMP levels) such that expression of an operably linked gene is induced. Accordingly, a functional variant of a CRE will contain suitable binding sites for CREB and/or AP1 (though other TFBS may also contribute). Suitable TFBS for CREB, AP1 and other TFs are discussed above.

The ability of CREB and/or AP1 (or any other TF) to bind to a given CRE can determined by any relevant means known in the art, including, but not limited to, electromobility shift assays (EMSA), binding assays, chromatin immunoprecipitation (ChIP), and ChIP-sequencing (ChIP-seq). In some embodiments the ability of CREB and/or AP1 to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861.

The ability of any given CRE to function as a forskolin-inducible element can be readily assessed experimentally by the skilled person. The skilled person can thus easily determine whether any given CRE or promoter (e.g. a variant of the specific forskolin-inducible promoters or CREs recited above) is functional (i.e. whether it can be considered to be a functional forskolin-inducible promoter or CRE, or if it can be considered to be a functional variant a specific promoters or CREs recited herein). For example, any given putative forskolin-inducible promoter can be linked to a gene (typically a reporter gene) and its properties when induced by forskolin are assessed. Likewise, any given CRE to be assessed can be operatively linked to a minimal promoter (e.g. positioned upstream of a MP) and the ability of the cis-regulatory element to drive expression of a gene (typically a reporter gene) when induced by forskolin is measured. Suitable constructs to assess the functionality activity of a forskolin-inducible CRE or a forskolin-inducible promoter, can easily be constructed, and the examples set out below give suitable methodologies. For example, any given putative forskolin-inducible CRE can be substituted in place of the incumbent CRE in any of the promoters Synp-FORCSV-10, Synp-FORCMV-09, Synp-FMP-02, Synp-FLP-01, Synp-RTV-017, Synp-RTV-019, or Synp-FORCYB1 discussed below, and linked to a reporter gene (e.g. luciferase or SEAP) and its inducibility and strength of expression upon induction can be assessed. In terms of inducibility, the level of induction of the reporter after cells, e.g. CHO-K1SV cells, are exposed to 18 pM forskolin for 5 h is suitably at least a 3-fold increase in expression, more preferably a 5-, 10-, 15-, 20-, 30-, or 50-fold increase in expression. In terms of strength of the promoter, upon induction (e.g. after cells, e.g. CHO-K1SV cells, are exposed to 18 pM forskolin for 5 h) the expression level of the reporter is at least 50% of that provided by the CMV-IE promoter (i.e. when compared to an otherwise identical vector in the same cells under the same conditions, but in which expression of the transgene is under control of CMV-IE rather than the forskolin inducible promoter). More preferably the expression level of the transgene is at least 75%, 100%, 150%, 200%, 300%, 400%, 500%, 750% or 1000% of that provided by the CMV-IE promoter. Likewise, any putative forskolin-inducible promoter can be substituted for promoters Synp-FORCSV-10, Synp-FORCMV-09, Synp-FM P-02, Synp-FLP-01, Synp-RTV-017, Synp-RTV-019, or Synp-FORCYB1 in the constructs of Examples 1, 2, 3 or 4 and inducibility and power assessed (the same conditions and preferred levels of inducibility and strength apply).

A hypoxia-responsive element (HRE) is a type of cis-regulatory element (CRE). More particularly, it is an inducible enhancer that is induced when cells in which the enhancer is present are subject to hypoxic conditions. HREs comprise a plurality of hypoxia-inducible factor biding sites (HBS). As described elsewhere, under hypoxic conditions the HIF heterodimer is formed in the cells and binds to HBSs, driving expression of genes containing them. This is well-described in the literature, see, for example, Wenger R H, Stiehl D P, Camenish G. Integration of oxygen signalling at the consensus HRE. Sci STKE 2005; 306:re12. [PubMed: 16234508]. More than one HRE can be present in the vectors of the present invention, thus providing a hypoxia-responsive cis-regulatory module (CRM).

Hypoxia-inducible factors (HIFs) are transcription factors that respond to hypoxia, i.e. a decrease in available oxygen in the cellular environment. In general, HIFs are vital to development. In mammals, deletion of the HIF-1 genes results in perinatal death. HIF-1 is of particular relevance to the present invention given its preeminent role in the hypoxia response, and thus it is preferred that the HREs of the present invention are targets for HIF-1. However, other HIFs (e.g. HIF-2 or HIF-3) may also bind to the HRE, and thus they are also of relevance. HIF-1, is a heterodimer composed of an α-subunit (HIF-1α) and a β-subunit (HIF-1β), the latter being a constitutively-expressed aryl hydrocarbon receptor nuclear translocator (ARNT). The alpha subunits of HIF are hydroxylated at conserved proline residues by HIF prolyl-hydroxylases, allowing their recognition and ubiquitination by the VHL E3 ubiquitin ligase, which labels them for rapid degradation by the proteasome. This occurs only in normoxic conditions. In hypoxic conditions, HIF prolyl-hydroxylase is inhibited, since it utilizes oxygen as a co-substrate. HIF-1, when stabilized by hypoxic conditions, upregulates several genes to promote survival in low-oxygen conditions. HIF-2 or HIF-3 are similarly formed from α- and β-subunits, as is well-described in the literature. The regulation of HIF1α and 2α by hypoxia is similar and both bind to the same core motif.

A hypoxia-inducible factor biding site (HBS) is a nucleic acid sequence that acts as a binding site for HIF. In endogenous genes, HBS comprise a conserved core sequence ([AG]CGTG, SEQ ID NO: 6) and highly variable flanking sequence.

It will be appreciated that the ability of a given HRE to function as a hypoxia-inducible enhancer is determined principally by the ability of the sequence to be bound by HIF (e.g. HIF-1) under hypoxic conditions such that expression of an operably linked gene is induced. Accordingly, a functional variant of an HRE will contain suitable binding sites for HIF. Generally, the presence of the consensus HBS is required.

The ability of HIF to bind to a given HRE can determined by any relevant means known in the art, including, but not limited to, electromobility shift assays (EMSA), binding assays, chromatin immunoprecipitation (ChIP), and ChIP-sequencing (ChIP-seq). In some embodiments the ability of HIF to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861. In a preferred method, the ability of a variant to bind HIF can be determined with pull down experiments.

For example, pull-down experiments can be carried out using biotinylated double-stranded probes with a variant HRE and a reference HRE. Using high stringency washing [6], the amount of HIF (e.g. assed in terms of the quantity of HIF-1a) from nuclear extract prepared from hypoxic cells can be compared between the variant HRE and reference HRE. Suitable methods are described in Stanbridge, et al. Rational design of minimal hypoxia-inducible enhancers Biochem Biophys Res Commun. 2008 Jun. 13; 370(4): 613-618 and Ebert BL, Bunn HF. Regulation of transcription by hypoxia requires a multiprotein complex that includes hypoxia-inducible factor 1, an adjacent transcription factor, and p300/CREB binding protein. Mol Cell Biol 1998; 18:4089-4096.

With regard to variants of any of the specific CRE or promoter sequences set out above, their functionality can be assessed by substituting the variant in place of the given CRE or promoter in the relevant construct of Example 1, 2, 3 or 4 and comparing the result for the construct comprising the variant against the results for the original construct. Preferably the functional variant maintains at least 50%, 60%, 70%, 80%, 90% or 100% of the inducibility of the parent construct (measured in terms of fold increase in expression as a result of induction, i.e. a 2-fold increase in expression of a reporter gene upon induction is considered to be 50% as inducible as a 4-fold increase). Preferably the functional variant maintains at least 50%, 60%, 70%, 80%, 90% or 100% of the expression strength of the reference construct upon induction. A functional variant also preferably results in a background expression level (i.e. absent any induction) that is no more than three times as high, preferably no more than twice as high, and preferably no more than 1.5 times as high when compared to the reference construct.

The ability of any given HRE to function as a hypoxia-inducible element can be readily assessed experimentally by the skilled person. The skilled person can thus easily determine whether any variant of the specific hypoxia-inducible promoters or HREs recited above remains functional (i.e. it is a functional hypoxia-inducible promoter or HRE, or if it can be considered to be a functional variant). For example, any given putative hypoxia-inducible promoter can be linked to a gene (typically a reporter gene) and its inducible properties when induced by hypoxia are assessed. Likewise, any given HRE to be assessed can be operatively linked to a minimal promoter (e.g. positioned upstream of a MP) and the ability of the cis-regulatory element to drive expression of a gene (typically a reporter gene) when induced by hypoxia is measured. Suitable constructs to assess activity of an HRE or a hypoxia-inducible promoter, can easily be constructed, and the examples set out below give suitable methodologies. For example, any given putative HRE can be placed in any of the promoters Synp-RTV-015, Synp-RTV-016, Synp-HYBT and Synp-HV3C, discussed below in place of the incumbent HRE, and linked to a reporter gene (e.g. luciferase or SEAP) and its inducibility and power can be assessed. For example, in terms of inducibility, the level of induction in after 5 h in cells when subjected to hypoxic conditions (e.g. moving from 20% oxygen to 5% oxygen) is suitably at least a 5-fold increase in expression, more preferably a 10-, 15-, 20-, 30-, or 50-fold increase in expression. For example, in terms of power, the level of expression in cells when subjected to hypoxic conditions (e.g. moving from 20% oxygen to 5% oxygen) is suitably at least 10% of the expression levels achieved by an otherwise identical constructs in which the CMV-IE promoter is used; more preferably at least 25%, 50%, 75%, 100%, 150% or 200% of the expression levels driven by CMV-IE.

Likewise, any putative hypoxia-inducible promoter can be substituted for promoters Synp-RTV-015, Synp-RTV-016, Synp-HYBT, Synp-HV3C in the constructs of examples 5, 6 or 7 and inducibility and power assessed (the same preferred levels of inducibility and power apply).

In one specific example, variants of HRE3 can be assessed by substituting the variant in place of HRE3 in the construct of HV3C or HYBT, and carrying out a suitable expression reporter assay, e.g. as described in Example 5, 6 or 7 and comparing the result for the construct comprising the variant to the results for the original HV3C or HYBT construct. Preferably the functional variant maintains at least 50%, 60%, 70%, 80%, 90% or 100% of the inducibility of the parent construct, and preferably the functional variant maintains at least 50%, 60%, 70%, 80%, 90% or 100% of the power of the parent construct.

Levels of sequence identity between a functional variant and the reference sequence can also be an indicator or retained functionality. High levels of sequence identity in the TFBSs or HBSs and spacing between the TFBSs or HBSs is of generally higher importance than sequence identity in the spacer sequences (where there is little or no requirement for any conservation of sequence).

As used herein, the term “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. A great many promoters are known in the art. The inducible promoters of the present invention typically drive almost none or low expression prior to being induced, and upon induction they drive a significantly higher level of expression (e.g. a 5, 10, 20, 50, 100, 150, 500, 700, or even 1000-fold increase in expression after induction).

The promoters of the present invention are synthetic promoters. The term “synthetic promoter” as used herein relates to a promoter that does not occur in nature. In the present context it typically comprises a synthetic CRE and/or CRM of the present invention operably linked to a minimal (or core) promoter. The CREs and/or CRMs of the present invention serve to provide forskolin inducible transcription of a gene operably linked to the promoter. Parts of the synthetic promoter may be naturally occurring (e.g. the minimal promoter or one or more CREs in the promoter), but the synthetic promoter as a complete entity is not naturally occurring.

As used herein, “minimal promoter” (also known as the “core promoter”) refers to a short DNA segment which is inactive or largely inactive by itself, but can mediate transcription when combined with other transcription regulatory elements. Minimum promoter sequence can be derived from various different sources, including prokaryotic and eukaryotic genes or can be synthetic. Examples of minimal promoters are discussed above, and include the synthetic MP1 promoter, cytomegalovirus (CMV) immediate early gene minimum promoter (CMV-MP) and the YB-TATA. A minimal promoter typically comprises the transcription start site (TSS) and elements directly upstream, a binding site for RNA polymerase II, and general transcription factor binding sites (often a TATA box).

As used herein, “proximal promoter” relates to the minimal promoter plus the proximal sequence upstream of the gene that tends to contain primary regulatory elements. It often extends approximately 250 base pairs upstream of the TSS, and includes specific TFBS. In the present case, the proximal promoter is suitably a naturally occurring proximal promoter that can be combined with one or more CREs or CRMs of the present invention. However, the proximal promoter can be synthetic.

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

“Synthetic” in the present application means a nucleic acid molecule that does not occur in nature. Synthetic nucleic acid expression constructs of the present invention are produced artificially, typically by recombinant technologies. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

“Transfection” in the present application refers broadly to any process of deliberately introducing nucleic acids into cells, and covers introduction of viral and non-viral vectors, and includes transformation, transduction and like terms and processes. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; whiskers-mediated transformation; and microprojectile bombardment (Klein et al. (1987) Nature 327:70).

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell), or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The term “operably linked”, “operably connected” or equivalent expressions as used herein refer to the arrangement of various nucleic acid elements relative to each such that the elements are functionally connected and are able to interact with each other in the manner intended. Such elements may include, without limitation, a promoter, an enhancer and/or a regulatory element, a polyadenylation sequence, one or more introns and/or exons, and a coding sequence of a gene of interest to be expressed. The nucleic acid sequence elements, when properly oriented or operably linked, act together to modulate the activity of one another, and ultimately may affect the level of expression of an expression product. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, cis-regulatory elements will typically be located immediately upstream of the promoter (although this is generally the case, it should definitely not be interpreted as a limitation or exclusion of positions within the nucleic acid expression cassette), but this needs not be the case in vivo, e.g., a regulatory element sequence naturally occurring downstream of a gene whose transcription it affects is able to function in the same way when located upstream of the promoter. Hence, according to a specific embodiment, the regulatory or enhancing effect of the regulatory element is position-independent.

A “spacer sequence” or “spacer” as used herein is a nucleic acid sequence that separates two functional nucleic acid sequences (e.g. TFBS, CREs, CRMs, minimal promoters, etc.). It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another.

“Cell culture”, as used herein, refers to a proliferating mass of cells that may be in either an undifferentiated or differentiated state.

“Consensus sequence”—the meaning of consensus sequence is well-known in the art. In the present application, the following notation is used for the consensus sequences, unless the context dictates otherwise. Considering the following exemplary DNA sequence:

A[CT]N{A}YR

A means that an A is always found in that position; [CT] stands for either C or T in that position; N stands for any base in that position; and {A} means any base except A is found in that position. Y represents any pyrimidine, and R indicates any purine.

“Complementary” or “complementarity”, as used herein, refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base. The degree of complementarity between nucleic acid strands has significant effects on the efficiency and strength of hybridization between nucleic acid strands.

As used herein, the phrase “transgene” refers to an exogenous nucleic acid sequence. In one example, a transgene is a gene sequence, a gene encoding an industrially or pharmaceutically useful compound, or a gene encoding a desirable trait. In yet another example, the transgene is an antisense nucleic acid sequence, wherein expression of the antisense nucleic acid sequence inhibits expression of a target nucleic acid sequence.

The terms “subject” and “patient” are used interchangeably herein and refer to animals, preferably vertebrates, more preferably mammals, and specifically include human patients and non-human mammals. “Mammalian” subjects include, but are not limited to, humans. Preferred patients or subjects are human subjects.

A “therapeutic amount” or “therapeutically effective amount” as used herein refers to the amount of expression product effective to treat a disease or disorder in a subject, i.e., to obtain a desired local or systemic effect. The term thus refers to the quantity of an expression product that elicits the biological or medicinal response in a tissue, system, animal, or human that is being sought by a researcher, veterinarian, medical doctor or other clinician. Such amount will typically depend on the gene product and the severity of the disease, but can be decided by the skilled person, possibly through routine experimentation.

As used herein, the terms “treat” or “treatment” refer to both therapeutic treatment and prophylactic or preventative measures. Beneficial or desired clinical results include, but are not limited to, prevention of an undesired clinical state or disorder, reducing the incidence of a disorder, alleviation of symptoms associated with a disorder, diminishment of extent of a disorder, stabilized (i.e., not worsening) state of a disorder, delay or slowing of progression of a disorder, amelioration or palliation of the state of a disorder, remission (whether partial or total), whether detectable or undetectable, or combinations thereof. “Treatment” can also mean prolonging survival as compared to expected survival if not receiving treatment.

As used herein, the terms “therapeutic treatment” or “therapy” and the like, refer to treatments wherein the object is to bring a subject's body or an element thereof from an undesired physiological change or disorder to a desired state, such as a less severe or unpleasant state (e.g., amelioration or palliation), or back to its normal, healthy state (e.g., restoring the health, the physical integrity and the physical well-being of a subject), to keep it at said undesired physiological change or disorder (e.g., stabilization, or not worsening), or to prevent or slow down progression to a more severe or worse state compared to said undesired physiological change or disorder.

As used herein the terms “prevention”, “preventive treatment” or “prophylactic treatment” and the like encompass preventing the onset of a disease or disorder, including reducing the severity of a disease or disorder or symptoms associated therewith prior to affliction with said disease or disorder. Such prevention or reduction prior to affliction refers to administration of the nucleic acid expression constructs, vectors, or pharmaceutical compositions described herein to a patient that is not at the time of administration afflicted with clear symptoms of the disease or disorder. “Preventing” also encompasses preventing the recurrence or relapse-prevention of a disease or disorder for instance after a period of improvement. In embodiments, the nucleic acid expression constructs, vectors, or pharmaceutical compositions described herein may be for use in gene therapy.

“Hypoxia”, “hypoxic” or related terms is a condition of low oxygen tension, typically in the range 1-5% O₂. Under such conditions eukaryotic cells respond through induction of various cellular responses, many of which are meditated by HIF. In a clinical context, hypoxic conditions is often found in the central region of tumours or other tissues due to poor vascularisation or disruption of blood supply. A CRE according to the eighteenth aspect of the present invention, a hypoxia-inducible promoter according to the nineteenth aspect of this invention or a gene therapy vector according to the twentieth aspect of this invention may be particularly useful in gene therapy where the tissue where therapy is required is hypoxic. This is often the case in cancer the central region of tumours and in the lymph nodes. “Normoxia” or “normoxic” is used to describe oxygen tensions between 10-20%, and “hyperoxia” for those above 20%. In the regions between 5 and 10% 02 cells may begin to show some moderate effects of hypoxia. In the present context, hypoxia can conveniently be induced by exposing cells to an oxygen tension of 5% or less.

### Introduction

The ATP derivative cyclic adenosine monophosphate (cAMP, cyclic AMP, or 3′,5′-cyclic adenosine monophosphate) is a second messenger important in many biological processes. Its main function is in intracellular signal transduction in many different organisms, conveying the cAMP-dependent pathway. This pathway has been well-studied, and it is reviewed in Yan, et al., MOLECULAR MEDICINE REPORTS 13: 3715-3723, 2016.

Activation of the adenylyl cyclase (also commonly known as adenyl cyclase and adenylate cyclase, abbreviated AC) drives a cascade that, via protein kinase A, leads to activation of the transcription factor CREB which binds specific TFBS (cAMPRE: TGACGTCA) to modulate gene expression.

Furthermore, AP1 is a TF complex (dimer) composed of variations of Fos and Jun proteins of which there are many forms. These proteins have a complex regulation pathway involving many protein kinases. The activation of AP1 sites (consensus sequence TGA[GC]TCA) by forskolin and other activators of adenylyl cyclase has been documented and is believed to be related to elevated cAMP levels ability to stabilise the protein c-Fos and upregulate its transcription. Therefore, AP1 site induced gene expression is an indirect effect of activation of the adenylyl cyclase. See, for example, Hess et al. Journal of Cell Science 117, 5965-5973 and Sharma and Richards, J. Biol. Chem. 2000, 275:33718-33728.

The present invention uses cAMPRE and the AP1 TFBS to generate novel synthetic CREs and promoters that are inducible by forskolin and other activators of adenylyl cyclase.

cAMPRE is the prototypical target sequence for CREB (Craig, et al., 2001).

AP1 is a consensus sequence of AP1 transcription factor binding sequences and AP1(1), AP1(2) are variants of the consensus sequence (Hess, et al., 2004) (Sharma & Richards, 2000).

Additionally, other TFBS were used in generating novel synthetic CREs and promoters that are inducible by activators of adenylyl cyclase.

HRE1 is a consensus sequence of hypoxia responsive elements from (Schödel, et al., 2011).

ATF6 is the consensus binding sequence for activated transcription factor 6 (ATF6) in cis-regulatory element Unfolded Protein Response Element (Samali, et al., 2010).

All promoters were placed upstream of the luciferase gene (Examples 1-3) or SEAP gene (Example 4).

To compare across experiments, the strength of the inducible promoters was compared to CMV-IE promoter, which was driving the same gene as the other constructs.

Luciferase readouts were normalised to β-galactosidase to produce normalised relative luminometer units (RLUs). β-galactosidase containing pcDNA6 plasmid was used as internal control for transfection efficiency (Thermofisher, V22020). β-galactosidase activity was measured as per manufacturer's instructions (Mammalian βGalactosidase Assay Kit, 75707/75710, Thermo Scientific) using 25 μl of lysate. 25 μl of lysate was transferred into a microplate well and mixed with 25 μl of β-galactosidase Assay Reagent, equilibrated to room temperature. The mixture was incubated at 37° C. for 30 min and absorbance measured at 405 nm.

The synthetic promoters were synthesised by GeneArt.

### Example 1

The forskolin-inducible promoters were initially used to drive expression of luciferase in the Huh7 human liver cell line and the C2C12 human muscle cell line. The promoter constructs were used to drive expression of luciferase in the PM-RQ plasmid. The tested promoters were synthesised directly upstream of the ATG of PM-RQ plasmid.

### Huh7 Transfection

### Materials

Huh7 cells which are a human liver cell line
DPBS: without CaCl₂, without MgCl₂ (Gibco, 14190-094)
DMEM (Sigma, D6546)
FBS (Sigma, F9665)
Pen-Strep (Sigma, P4333)
Promega Fugene-HD (E2311)
Forskolin (Sigma, F6886)
NKH477 (Sigma, N3290)
LARII (Dual Luciferase Reporter 1000 assay system, Promega, E1980)
48 well plates flat bottom (353230, Corning)
Trypsin-EDTA (25200-056, Gibco)
T25 & T75 flask (353108, 353136, Corning)
15 ml Falcon tubes (430791, Corning)
Stripettes 5 ml, 10 ml and 25 ml (4101,4051,4251, Corning)

### Day 1

Cells were seeded onto a 48 well plate at a density of 25,000 cells/300 μl

### Day 2

On the day of transfection, DNA to be transfected was diluted to a 100 ng/μl stock solution. DNA to be transfected was the RTV-017, RTV-019 or CMV-IE promoter driving luciferase in the PM-RQ plasmid.
Per 48 well transfection, 45 ng of DNA was mixed with 4.1 μl of Optimem medium.
0.5 μl of Fusion HD was mixed with 4 μl of Optimem medium.
These 2 solutions were mixed and incubated at room temperature for 15 minutes.
The final solution was then added to the well drop wise.
3 hrs after transfection the inducer was added to the appropriate wells at the indicated concentration. Inducer was forskolin at 20 pM.

### Day 3

24 hrs after induction, the luciferase activity was measured as described below.

### C2C12 Transfection

### Materials:

C2C12 cell line which is a muscle cell line (91031101, Sigma, lot number 15D022)
DPBS without CaCl₂), without MgCl₂ (14190-094, Gibco)
Horse Serum (16050-122, Gibco)
Viafect (E4981, Promega)
DMEM (high glucose, D6546, Sigma)
DMEM (high glucose, no sodium pyruvate, 11960-044, Gibco)
Penicillin-streptomycin Solution (15140-122, Gibco)
FBS (Gibco10500-064, Lot number 08Q2771K)
GlutaMax (35050-038, Gibco)
48 well plates flat bottom (353230, Corning)
Trypsin-EDTA (25200-056, Gibco)
T25 & T75 flask (353108, 353136, Corning)
15 ml Falcon tubes (430791, Corning)
Stripettes 5 ml, 10 ml and 25 ml (4101,4051,4251, Corning)

### Media Preparation:

Media was prewarmed at 37° C. before use
C2C12 Complete Medium was used to grow the cells before differentiation. To prepare 50 ml of complete medium, 44 ml DMEM (high glucose, D6546, Sigma), 0.5 ml GlutaMAX, 0.5 ml Penicillin-streptomycin solution and 5 ml FBS was added.
C2C12 Transfection Medium was used for transfection of the C2C12 cells. To prepare 50 ml of transfection medium, 49.5 ml DMEM (high glucose, D6546, Sigma) and 0.5 ml GlutaMAX was added.
C2C12 Differentiation Medium was used to differentiate the C2C12 cells. To prepare 50 mil of differentiation medium, 48.5 ml DMEM (high glucose, no sodium pyruvate, 11960-044, Gibco), 1 ml Horse Serum (Heat Inactivated) and 0.5 GlutaMAX was added.

### Transfection:

C2C12 cells were maintained at 37° C., 5% CO₂. During transfection, the C2C12 cells were checked daily using an inverted microscope. C2C12 cell line was only used up to passage 12 as beyond this passage, the cells progressively lose their differentiation ability.
Day 1—Plating C2C12 cells for transformation
C2C12 Complete Medium, trypsin EDTA, and PBS were prewarmed at 37° C.
C2C12 cells grown in complete medium were aspirated, washed with 5 ml DPBS, aspirate and 1.0 ml Trypsin/EDTA solution (for a T75 flask) was added
The cells were left at 37° C. for 3 to 5 mins, until the cells detach. Detachment was monitored under the microscope.
4 ml C2C12 Complete Medium was added to inactivate Trypsin and the cells were transferred into a 15 ml tube.
The cells were centrifuged for 3 mins at 250 RCF at room temperature.
The supernatant was aspirated
The cells were resuspended by gentle pipetting up and down in 6 ml C2C12 Complete Medium
The cells were counted in a haemocytometer by using trypan blue (Thermofisher, 15250061) to determine the how many of the cells were alive. Trypan blue stains dead cells in blue and this allows for them not to be counted. The cells were resuspended into trypan blue in ration of 1:10, e.g. 10 μl cells and 90 μl trypan blue and counted using a haemocytometer.
The cells were seeded in complete medium at a density dependent on the passage number. For low passage numbers (passage 4-6), the cells were seeded at a density of 45000 cells/well. For medium passage number (passage 7-9), the cells were seeded at a density of 40000 cells/well. For high passage number (passage 10-12), the cells were seeded at a density of 38000 cells/well. Different cell densities were used at different passage numbers as the cells differentiate from myoblasts to myotubes if seeded at the wrong density. The abovementioned densities are experimentally determined for efficient differentiation and transfection.

### Day 2—Transfection

24 hours after seeding, the cells were transfected
C2C12 transfection medium was prewarmed at 37° C.
The DNA stock to be transfected was diluted to 100 ng/μl in deionized water (MilliQ grade). Following dilution, the concentration of the diluted DNA sample was checked by measuring absorbance at 260 nm to ensure id did not deviate more than 10% from the desired 100 ng/μl. DNA to be transfected was the RTV-019 or CMV-IE promoter driving luciferase in the PM-RQ plasmid.
The media of the C2C12 cells which were seeded in the 48 well plate the previous day was changed to C2C12 Transfection Medium (300 μl of medium/well).
DNA/transfection reagent complexes were prepared following the manufacturer's instructions (Viafect—E4981, Promega). For a 48 well plate, 30 μl of the following mixture was prepared per well: 26.1 μl C2C12 plain medium (DMEM high glucose, D6546, Sigma—no additives), 0.3 pg DNA (3 μl of a DNA dilution adjusted to 100 ng/μl) and 0.9 μl Viafect. The DNA was initially added to the DMEM plain medium and incubated for 5 minutes. The Viafect was then added and the mixture mixed. The mixture was incubated for 20 minutes at room temperature to allow DNA and the transfection reagent to complex.
30 μl of transfection complexes was added per well and the plate was gently swirled so that the reaction mix is evenly spread in the well and the cells were incubated at 37° C., 5% CO₂.
   Day 3—Switch to differentiation media
24 hours after transfection, the media of the C2C12 cells was changed to differentiation media

### Day 6.5—Induction

20 pM Forskolin was added to the appropriate wells and the cells were incubated for 24 hrs before reading.
   Day 7.5—Luciferase assay
5.5 days after transfection, the luciferase assay was performed as described below

### Measurement of Luciferase Activity

Luciferase activity was measured using LARII (Dual Luciferase Reporter 1000 assay system, Promega, E1980)
24 hours after induction, the media was removed from the cells
The cells were washed once in 300 μl of DPBS.
Cells were lysed using 100 μl of passive lysis buffer and incubated with rocking for 15 minutes.
The cell debris was pelleted by centrifugation of the plate at max speed in a benchtop centrifuge for 1 min
For luciferase, 10 μl sample was transferred into white 96-well plate and luminescence measured by injection of 50 μl of LARII substrate

### Results

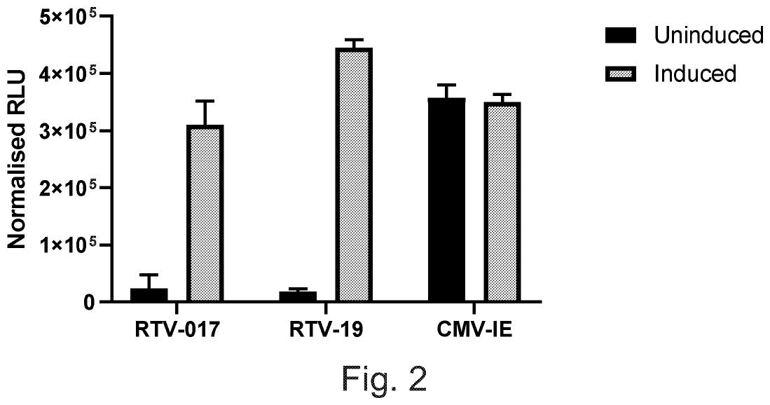

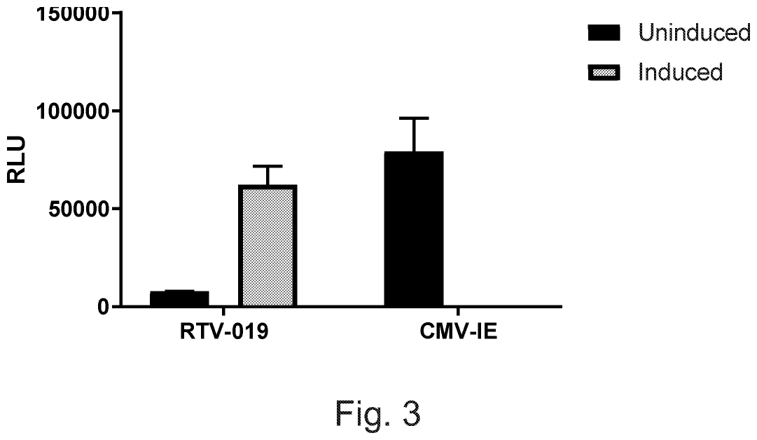

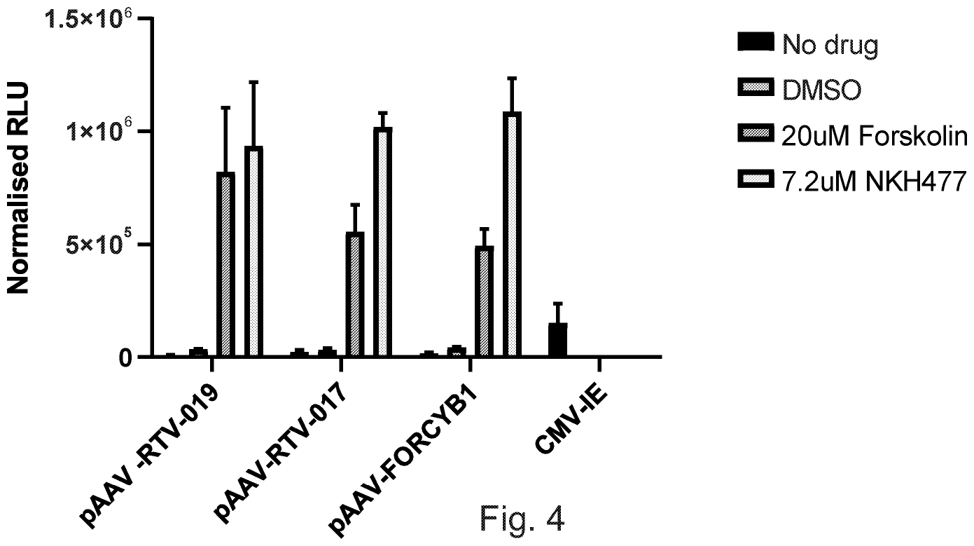

RTV-017 and RTV-019 were transiently transfected into the human liver cell line Huh7 (FIG. 2). RTV-019 was transiently transfected into the human muscle cell line C2C12 (FIG. 3). The activity of the promoters was assessed using luciferase activity. The luciferase activity was measured with and without the presence of 20 pM forskolin.

Both the RTV-017 and RTV-019 promoters in Huh7 and the RTV-019 promoter in C2C12 have low activity before the addition of forskolin. Upon addition of forskolin, the promoters have a much higher activity. In liver cells the promoters are induced 13 and 24-fold respectively whereas in muscle cells the promoter is only activated 6-fold.

### Example 2

The forskolin-inducible promoters RTV-017, RTV-019 and FORCYB1 were then used to drive expression of luciferase in a pAAV vector in the Huh7 human liver cell line. The constructs were cloned into pAAV by Gibson assembly. This experiment was performed to investigate the effect of the Inverted Terminal Repeats (ITRs) on the activity of the promoters. This was done as we have observed interference from the AAV ITRs in other projects.

The cells were seeded, transfected and induced as described above. Luciferase expression was measured as described above. In this experiment, the cells were induced by using 20 pM forskolin or 7.2 pM of the water soluble derivative NKH477. Similarly, to the activity pattern observed in FIGS. 2 and 3, the promoters appeared to show low background activity before induction and the high level of luciferase activity upon induction. The induction for RTV-017 was 23 and 44-fold for with NKH477 and forskolin respectively. The induction for RTV-019 was 86 and 98-fold with NKH477 and forskolin respectively. The induction for FORCYB1 was 30 and 65-fold with NKH477 and forskolin respectively. This indicates that all promoters have good inducibility both with forskolin and the water soluble derivative NKH477. Moreover, the ITRs do not appear to substantially affect the promoter activity. This indicates that the promoters might be useful in gene therapy as transfection of Huh7 with pAAV plasmid is a good indicator of the promoter's performance in AAV viral particles.

### Example 3

The forskolin-inducible promoters RTV-017, RTV-019, FORCSV-10, FORCYB-001, FOR-CMV-009, FMP-02 and FLP-01 were then used to drive expression of luciferase in a PM-RQ vector in the suspension cell line HEK293-F. The forskolin-inducible promoters RTV-017, RTV-019, FORCSV-10, FORCYB-001, FOR-CMV-009, FMP-02 and FLP-01 were also used to drive expression of luciferase in a PM-RQ vector in the suspension cell line CHO-K1SV. The tested promoters were synthesised directly upstream of the ATG of PM-RQ plasmid and the suspension cell lines were transiently transfected with the PM-RQ plasmid.

### Transfection of HEK293-F Cells

40 ml of cells were grown in a 250 ml vented Erlenmeyer flask (Sigma-Aldrich CLS431144) at 37° C., 20% O₂, 8% CO₂ with agitation at 100 rpm. Cells were seeded as described in the manufacturer's instructions (300,000 cells/ml). HEK293-F were obtained from Thermofisher, R79007.

One day before transfection, the cells were counted using a haemocytometer and split to 500,000 cells/ml.

On the day of transfection, the cells are seeded to 1,000,000 cells/ml in 500 μl of appropriate medium (Freestyle 293 expression medium, 12338002) in a 24 well plate. 0.625 pg of DNA per well was then added to 10 μl of OptiMem medium (Thermofisher; 11058021) and incubated for 5 minutes at room temperature.

Concurrently, 0.625 μl of Max reagent (Thermofisher, 16447100) was made up to 10 μl by addition of OptiMem and incubated for 5 minutes at room temperature. After this incubation, both mixes were added to the same tube and incubated at room temperature for 25-30 minutes. The DNA/Max reagent mix (20 μl/well) was then added directly to the cells and the cells incubated at 37 C, 8% CO₂ with agitation at 100 rpm.

24 hours after transfection, the promoters were induced by addition of 20 pM forskolin and luciferase activity was measured 0, 3, 5 and 24 hours after induction. Luciferase activity was measured as previously described.

### Transient Transfection of CHO-K1SV Cells

40 ml of cells were grown in a 250 ml vented Erlenmeyer flask (Sigma-Aldrich CLS431144) at 37° C., 20% O₂, 8% CO₂ with agitation at 100 rpm. Cells were seeded at 300,000 cells/ml.

One day before transfection, the cells were counted using a haemocytometer and split to 500,000 cells/ml.

On the day of transfection, the cells are seeded to 1,000,000 cells/ml in 500 μl of appropriate medium (Thermofisher, CD-CHO 10743029) in a 24 well plate. 0.625 pg of DNA per well was then added to 10 μl of OptiMem medium (Thermofisher; 11058021) and incubated for 5 minutes at room temperature.

Concurrently, 0.625 μl of Freestyle Max reagent (Thermofisher, 16447100) was made up to 10 μl by addition of OptiMem and incubated for 5 minutes at room temperature. After this incubation, both mixes were added to the same tube and incubated at room temperature for 25-30 minutes. The DNA/Max reagent mix (20 μl/well) was then added directly to the cells and the cells incubated at 37 C, 8% CO₂ with agitation at 100 rpm.

The promoters were induced by addition of 20 pM forskolin and luciferase activity was measured after 24 hours. Luciferase activity was measured as described before.

### Results

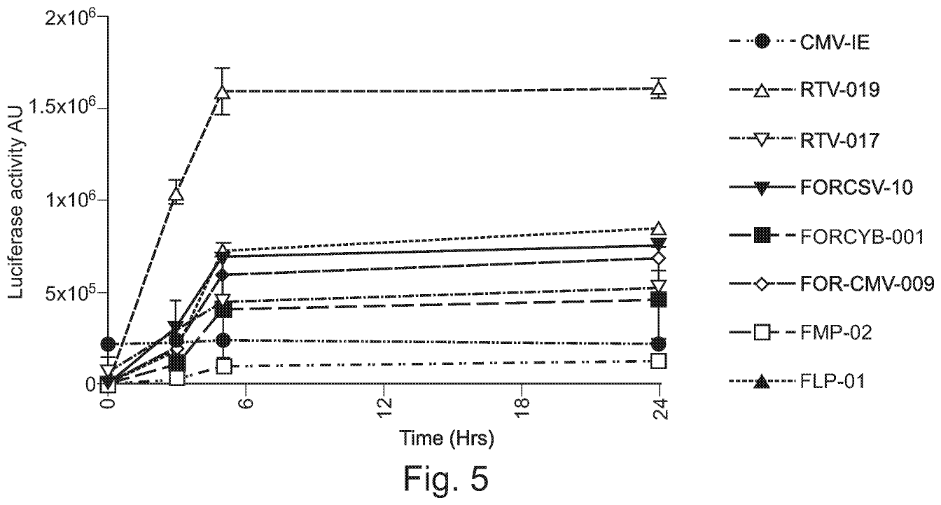

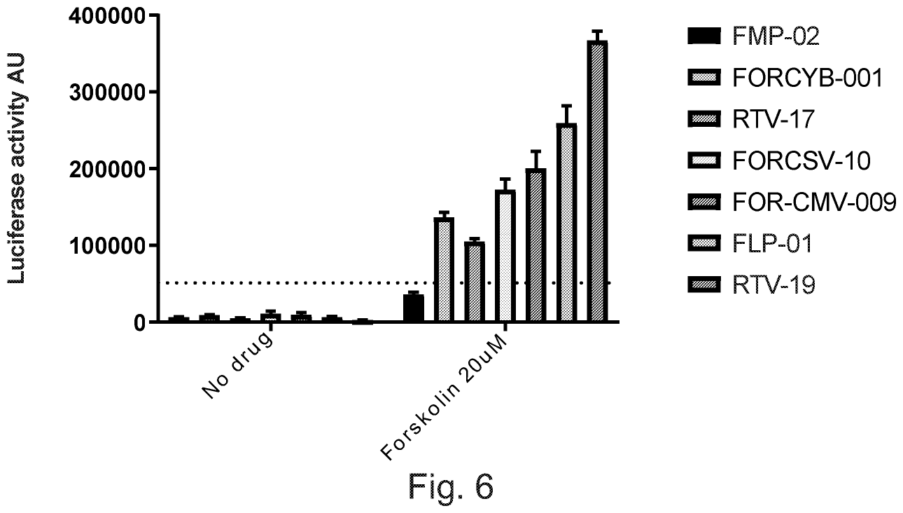

The forskolin-inducible promoters were used to drive expression of luciferase in the suspension cell line HEK293-F (FIG. 5) and CHO-K1SV (FIG. 6).

In the time course after induction, the promoters show a low background with a rapid increase in activity with maximal activity seen after 5 hrs. This activity is maintained until 24 hrs. Fold induction for the promoters varies from 50 to 100-fold, with FMP-02 being the weakest and RTV-019 being the strongest. The dynamic range of the promoters is also very wide with a 10-fold range at maximal activity. These results show that the promoters may be promising in bioprocessing applications due to their tight control and wide dynamic range (the ratio of the strongest promoter strength to the weakest promoter strength).

### Example 4

The forskolin-inducible promoters were then tested in a stably transfected CHO-GS-KSV1 cell line.

### Generating CHO-GS-KSV1 Stable Cell Lines

### Materials

CD-CHO media (Life technologies, CAT #10743029)
Corning 125 mL Polycarbonate Erlenmeyer Flask with Vent Cap (CAT #734-1885).
Gene Pulser® Electroporation Cuvettes, 0.4 cm gap (BioRad, CAT #165-2088)
Gene Pulser Xcell Total System (BioRad, CAT, #1652660)
GS-vector DNA (40 pg in 100 μL TE buffer) linearised with Sca1
Suspension cultures of CHOK1SV GS-KO host cells.

Cell suspensions of 6×10⁵ cells per mL were incubated on an orbital shaker set at 8% CO₂, 20% O₂, 37° C., 85% relative humidity and 140 rpm overnight. 2.86×10⁷ cells were centrifuged at 200 g for 3 minutes. Media was then aspirated and cell pellet resuspended in 2 mL fresh CD-CHO media to obtain a concentration of 1.43×10⁷ cells per mL. 700 μL of cell suspension was added to each of two electroporation cuvettes each containing 40 g of linearised DNA in 100 μL of sterile TE buffer (Thermofisher, 12090015). Each cuvette was electroporated, delivering a single pulse of 300V, 900 pF with resistance to infinity. Immediately after delivery of pulse electroplated cells were transferred to Erlenmeyer 125 mL flask containing 20 mL of CD-CHO media pre-warmed to 37° C. Electroporated cells from two cuvettes are combined into a single 125 mL flask to generate one pool of cells. Cells are cultured on an orbital shaker set at 8% CO₂, 20% O₂, 37° C., 85% relative humidity and 140 rpm. Cells are transferred to fresh CD-CHO media 24 hours after transfection and cell cultures monitored and given fresh CD-CHO media every 2-4 days. Usually after about 10-14 days cell numbers will be high enough to start passaging.

The transfected DNA was the pXC-17.4 expression vector (Lonza Biologics plc) where one of the promoter constructs (or a control promoter (CMV-IE) have been cloned upstream of the secreted alkaline phosphatase (SEAP) gene, which had been cloned into the multiple cloning site within the vector expression cassette. Promoters were closed into the pXC-17.4 vector using Gibson assembly. The pXC-17.4 expression vector is designed for making stable cell lines in the CHO-GSK1SV cell line as it contains the glutamate synthase gene which has been knocked out of the cell line. Therefore, selection of cells in glutamine drop-out medium will select for cells that have stably integrated the plasmid.

The responsiveness of the RTV-019 promoter to different concentrations of forskolin and NJH477 was tested in the stably transfected CHO-GS-KSV1 cell line. To this end, the stably transfected CHO-GS cells were seeded at 500,000 cells/ml and allowed to grow for 24 hrs. At this point the cells were induced by addition of the respective concentrations of forskolin and NKH477 and SEAP expression was measured as described below after 24 hours.

The promoter activity and inducibility of FMP-02, FORCYB-001, RTV-017, FORCSV-10, FOR-CMC-009, FLP-01 and RTV-019 were also tested in the stably transfected CHO-GS-KSV1 cell line. To this end, the stably transfected CHO-GS cells were seeded at 500,000 cells/ml and allowed to grow for 24 hrs. At this point the cells were exposed to 20 pM forskolin or 7.2 pM NKH477 for 24 hours. After this point, the SEAP activity in the medium was assessed as described below.

### SEAP Assay

SEAP Reporter Gene Assay, chemiluminescent (Roche, CAT #11 779 842 001) was used to measure SEAP activity as per the manufacturer's protocol. All reagents and samples were fully pre-equilibrated at room temperature. Culture supernatant was collected from the stably transfected CHO-GS at the specific time points (0 h and 24 h). The supernatant was diluted 1:4 in dilution buffer and heat treated at 65° C. for 30 minutes. The heat-treated sample was then centrifuged for 30 s at maximum speed. 50 μl of the heat-treated sample was then added to 5 μl of inactivation buffer and incubated for 5 min at room temperature. 50 μl of substrate reagent was then added and incubated for 10 min at room temperature. The signal is then read at 477 nm and compared to a calibration curve. SEAP expression was normalized against cell number to ensure that the increase in activity was not due to increased cell numbers and was true induction.

### Results

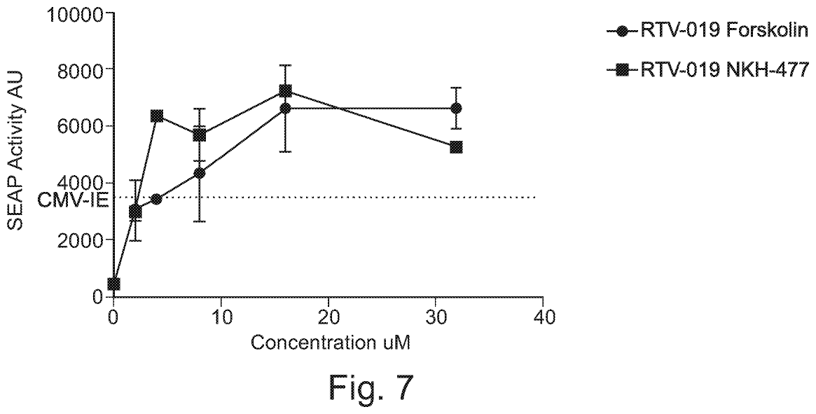

The response of the promoter RTV-019 to increasing concentrations of forskolin and NKH477 in the stably transfected CHO-GS-KSV1 cell line is shown in FIG. 7. The cell line was grown for 24 hrs then exposed to either 2, 5, 9, 18 or 32 pM of forskolin or NKH477. All concentrations induced the promoters but the optimal was 18 pM for forskolin and 8 pM for NKH477. This correlates well with the concentrations reported in the literature wherein the increase of the cAMP level by forskolin and NKH477 was measured.

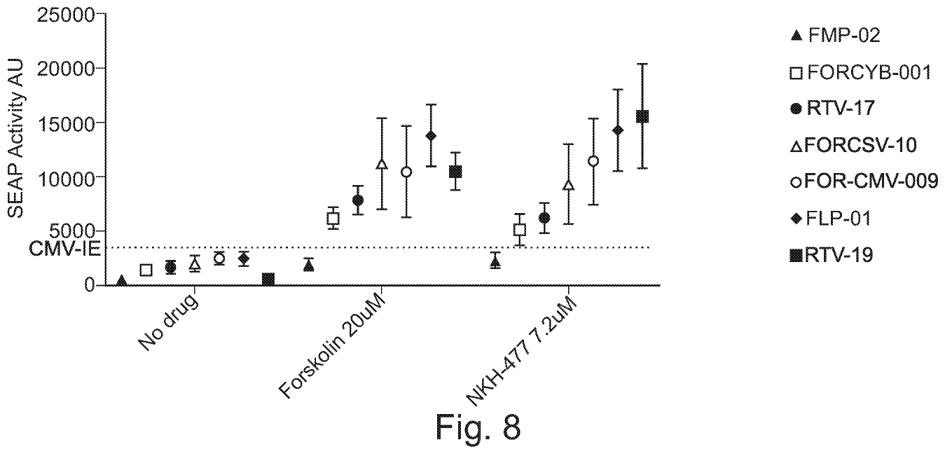

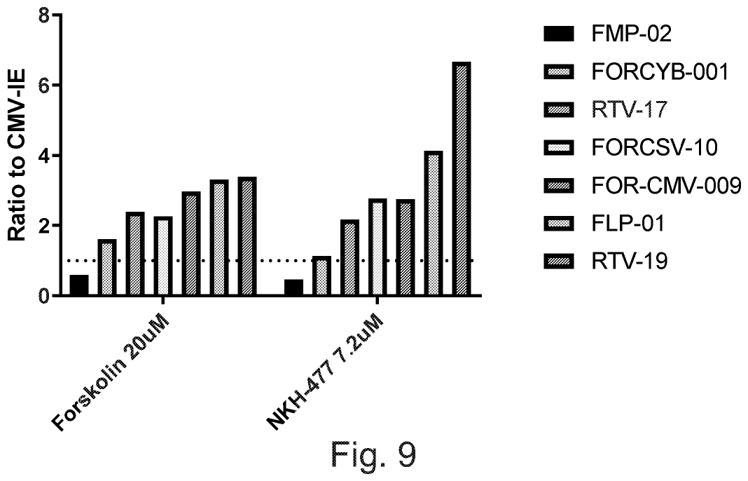

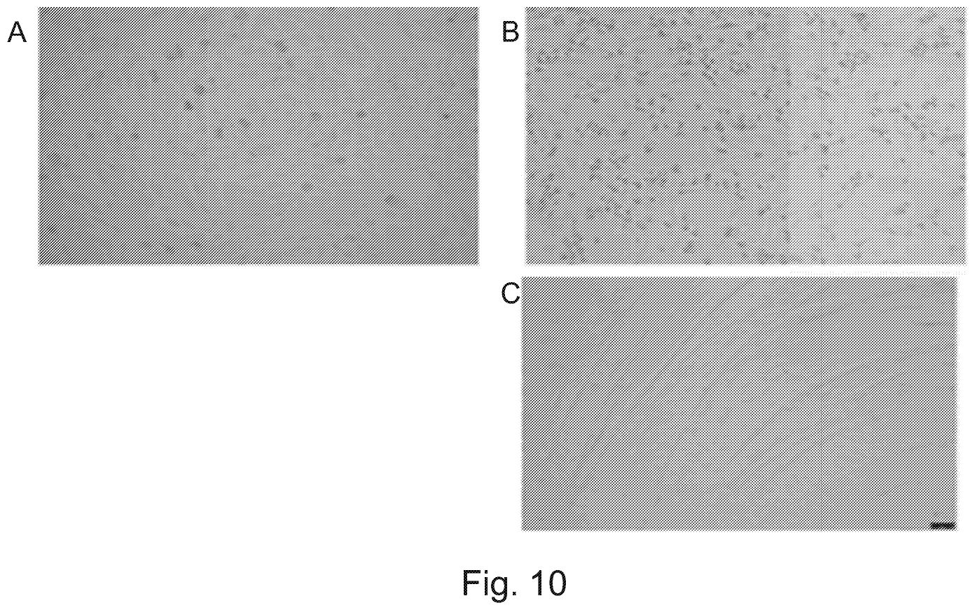

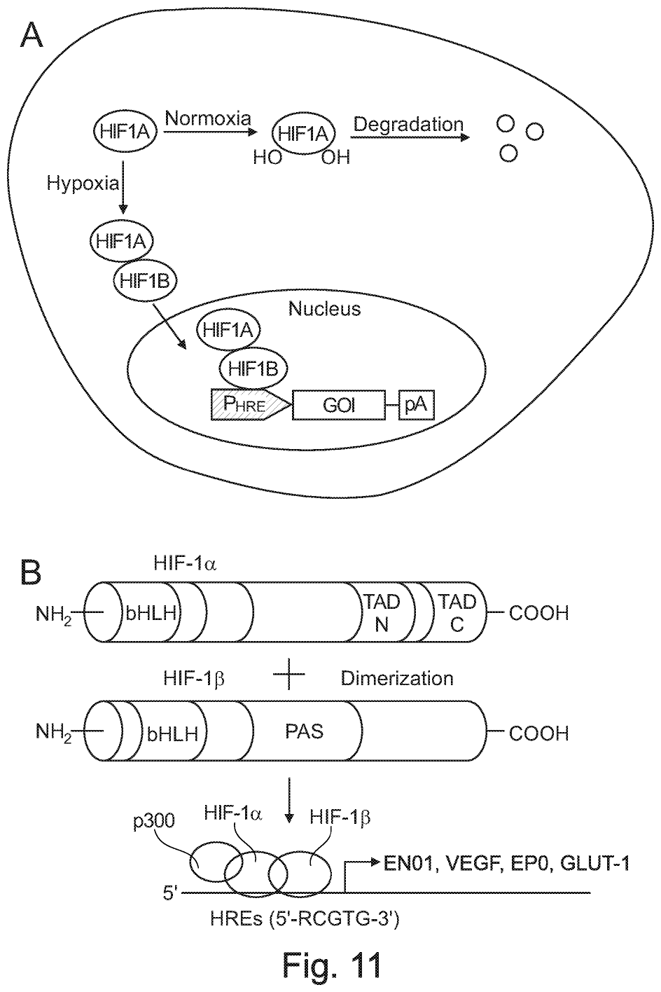

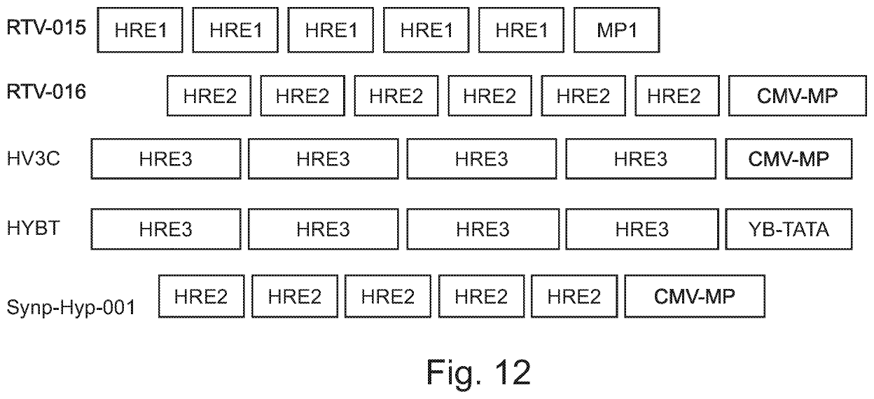

The response of the promoters to 20 pM forskolin or 7.2 pM NKH477 in the stably transfected CHO-GS-KSV1 cell line is shown in FIGS. 8 and 9. FIG. 8 shows the activity of the promoters and FIG. 9 shows the maximum activity reached by each promoter compared with CMV-IE. All promoters show increased expression upon addition of either forskolin or NKH477. As before, FMP-02 is the weakest and RTV-019 is the strongest. There is up to 35-fold induction seen in this system with a range of 6-fold between the weakest and strongest expressors. In this experiment, it appears that NKH477 was a slightly more potent inducer of the promoters. NKH477 is a preferred inducer for bioprocessing because it is water soluble and it will be easier to wash it away during purification steps.

Overall these promoters show great promise for use in both bioprocessing (CHO-K1SV, HEK293-F) and gene therapy (Huh7, pAAV, C2C12). The promoters are robust in multiple cell types showing good inducibilty and strength while maintaining low background.

### Hypoxia and HIF

The importance of the HIF signalling cascade is shown by knockout studies in mammals which leads to perinatal death. This is due to its role in the development of the vascular system and chondrocyte survival. In addition, HIF1 plays a central role in human metabolism as it is linked with respiration and energy generation. Furthermore, the cascade mediates the effects of hypoxia by upregulating genes important for survival in such conditions. For example, hypoxia promotes the formation of blood vessels, which is a normal response essential in development. However, in cancer, hypoxia can also lead to the vascularisation of tumours.

The main response element for the sensing and upregulation of genes involved in hypoxia stress response is the transcriptional complex HIF1. This complex is highly conserved across eukaryotes and is formed by the dimerization of 2 subunits, α and β. The β-subunit is constitutively expressed and is an aryl hydrocarbon receptor nuclear translocator (ARNT) essential for translocation of the complex to the nucleus. Both the α and β-subunits belong to the basic helix-loop-helix family of transcription factors and contain the following domains:
N-terminal: bHLH domain for DNA binding
Central heterodimerization domain: Per-ARNT-Sim (PAS) domain
C-terminal: recruits transcriptional coregulatory proteins

### HIF Mechanism of Action

Under normoxic conditions HIF1α subunits are hydroxylated at conserved proline residues. This hydroxylation by HIF prolyl-hydroxylases targets the subunits for recognition and ubiquitination by the VHL E3 ubiquitin ligase and subsequent degradation by the proteasome. However, under hypoxic conditions, oxygen limitation inhibits the HIF prolyl-hydroxylase as oxygen is an essential co-substrate for this enzyme. Once stabilised, HIF-1α subunits can heterodimerise with HIF-1β subunits and translocate to the nucleus where they can upregulate the expression of a number of genes. This is achieved by the HIF complex's binding to HIF-responsive elements (HREs) in promoters that contain the HBS sequence NCGTG (SEQ ID NO: 5) (where N is preferably either an A or G) or its reverse complement. The genes upregulated by the HIF1 complex are involved in central metabolism, such as glycolysis enzymes which allow ATP synthesis in an oxygen-independent manner, or in angiogenesis such as vascular endothelial growth factor (VEGF).

### Pseudohypoxia

There are alternative ways to activate the HIF1 complex. Mutations to SDHB, one of four protein subunits forming succinate dehydrogenase, cause build-up of succinate by inhibiting electron transfer in the succinate dehydrogenase complex. This excess succinate inhibits HIF prolyl-hydroxylase, stabilizing HIF-1α.

NF-κB can also directly modulate HIF1 regulation under normoxic conditions. It is believed that NF-κB can regulate basal HIF-1α expression as increased HIF-1α levels was correlated with increased NF-κB expression.

### Hypoxia Responsive Elements

Hypoxia-responsive elements tend to have a conserved HIF1 binding consensus sequence, NCGTG (SEQ ID NO: 5), where N is preferably either an A or G (Schödel, et al., 2011, Blood. 2011 Jun. 9; 117(23):e207-17.). The flanking sequence of this is notoriously variable but still contributes to the activity of the promoter.

The following exemplary HIF binding sequences (HBS) are used in the following examples:
HRE1 (ACGTGC (SEQ ID NO: 8)) which is a variant of the consensus sequence ([AG]CGTG, SEQ ID NO: 6) found in HIF binding sites of hypoxia-responsive elements (Schödel, et al., 2011).
HRE2 (CTGCACGTA (SEQ ID NO: 7)) was described as a superior and highly active hypoxia-inducible motif (Kaluz, et al., 2008, Biochem Biophys Res Commun. 2008 Jun. 13; 370(4):613-8).
HRE3 (ACCTTGAGTACGTGCGTCTCTGCACGTATG (SEQ ID NO: 9)) was described as a strongly induced element (Ede, et al., 2016, ACS Synth. Biol., 2016, 5 (5), pp 395-404). HRE3 is a composite HBS which comprises both HRE1 and HRE2 and it was hypothesised that it may be possible to increase the strength of induction by using this element.

Synthetic promoters comprising these HBS sequences were prepared and tested as described below.
Synp-HYP-001 construct (SEQ ID NO: 83) comprises 4 HRE2 elements without spacers, a spacer of 32 base pair length between the core of the last HRE2 and the TATA box of the CMV minimal promoter. This construct was designed with suboptimal spacing between the HRE2 elements and between the last HBS and the minimal promoter, and it was predicted to have relatively low inducibility and strength of expression.
Synp-RTV-015 construct (SEQ ID NO: 81) comprises 5 HRE1 elements spaced apart by 40 bp spacers, followed directly by a synthetic minimal promoter TATA box (MP1). This promoter was designed to be only weakly induced by hypoxia by its suboptimal spacing of 40 bp between the HRE1 elements and a spacing of 36 bp from the core of the last HRE1 HBS to the TATA box of MP1.
Synp-RTV-016 construct (SEQ ID NO: 82) comprises 6 HRE2 elements spaced apart by 20 bp spacers, with length of 65 base pairs between the core of the last HRE2 HBS and the TATA box of the CMV minimal promoter. This promoter was designed to be of medium strength, stronger than RTV-015 but weaker than HYBT and HV3C, as the spacing between the HBSs and the minimal promoter is suboptimal.
Synp-HYBT construct (SEQ ID NO: 84) comprises 4 HRE3 elements spaced apart by 9 bp spacer (meaning a spacing of 21 bp between cores of adjacent HRE3 elements, and an internal spacing of 8 bp between the two core sequences of the HRE3 element), and a spacing of 29 base pairs between the core of the last HRE3 and the TATA box of the YB-TATA minimal promoter. This construct was designed to have strong activity.
Synp-HV3C construct (SEQ ID NO: 85) comprises 4 HRE3 elements spaced apart by 9 bp spacer (meaning a spacing of 21 bp between cores of adjacent HRE3 elements, and an internal spacing of 8 bp between the two core sequences of the HRE3 element), and a spacing of 25 base pairs between the core of the last HRE3 and the TATA box of the CMV minimal promoter. This construct is very similar to HYBT but differs in the minimal promoter used. This construct was designed to have strong activity.

All promoters were placed upstream of the luciferase gene (Examples 1 and 2) or SEAP gene (Example 3).

To compare across experiments, the strength of the inducible promoters was compared to CMV-IE promoter, which was driving the same gene as the other constructs.

The synthetic promoters were synthesised by Geneart. The promoter constructs were used to drive expression of luciferase in the pMQ plasmid, unless otherwise stated.

### Example 5

The constructs were initially used to drive luciferase expression in HEK293-F and HEK293-T in hypoxia.

### Transfection of HEK293-F Cells in 24 Well Format

40 ml of cells were grown in a 250 ml vented Erlenmeyer flask (Sigma-Aldrich CLS431144) at 37° C., 20% O₂, 8% CO₂ with agitation at 100 rpm. Cells were seeded as described in the manufacturer's instructions (300,000 cells/ml). HEK293-F were obtained from Thermofisher, R79007.

One day before transfection, the cells were counted using a haemocytometer and split to 500,000 cells/ml.

On the day of transfection, the cells are seeded to 1,000,000 cells/ml in 500 μl of appropriate medium (Freestyle 293 expression medium, 12338002) in a 24 well plate. 0.625 pg of DNA per well was then added to 10 μl of OptiMem medium (Thermofisher; 11058021) and incubated for 5 minutes at room temperature.

Concurrently, 0.625 μl of Max reagent (Thermofisher, 16447100) was made up to 10 μl by addition of OptiMem and incubated for 5 minutes at room temperature. After this incubation, both mixes were added to the same tube and incubated at room temperature for 25-30 minutes. The DNA/Max reagent mix (20 μl/well) was then added directly to the cells and the cells incubated at 37 C, 8% CO₂ with agitation at 100 rpm. The transfected DNA was one of the vectors where the promoter constructs (RTV-015, HYBT, RTV-016, HV3C, Synp-HYP-001) or a control promoter (CMV-IE) were used to drive luciferase expression and β-galactosidase containing pcDNA6 plasmid. The β-galactosidase containing plasmid was used as internal control for transfection efficiency (Thermofisher, V22020).

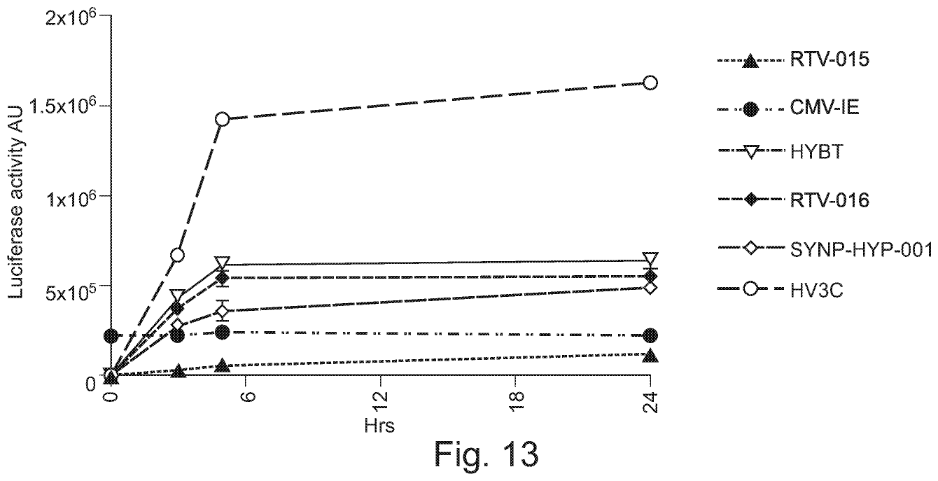

After transfection the cells were incubated for 24 hrs in normoxia conditions (20% oxygen) before being switched to a gas mix of 5% oxygen, 10% carbon dioxide and 85% nitrogen (hypoxia). This was achieved by gas displacement in a sealed hypoxia chamber. Induction of the promoters was assessed by using luciferase activity after 3, 5 and 24 hrs in hypoxia. These results are shown in FIG. 13.

### Transfection of HEK293-T Cells

HEK293-T are seeded at a density of 20%. Once they reached a confluence between 60 and 80%, the media is changed with DMEM (#21885-025—Thermo Scientific) supplemented with 10% FBS (Gibco, 26140). After 3 hours, the cells were transfected by a transfection mix. The transfection mix is prepared by adding DNA (2 pg per 6 well plate) and PEI 25 kDA (#23966-1—Polyscience) in a ratio of 1:3 in sterile DPBS (#14190169—ThermoFisher Scientific). After mixing, the transfection mix is incubated at room temperature for 30 minutes and then added directly to the cells. After 16 to 18 h post transfection, the media is changed to DMEM+2% FBS. The transfected DNA was one of the vectors where the promoter constructs (RTV-015, HYBT, RTV-016, HV3C) or a control promoter (CMV-IE) were used to drive luciferase expression and β-galactosidase containing pcDNA6 plasmid. The β-galactosidase containing plasmid was used as internal control for transfection efficiency (Thermofisher, V22020).

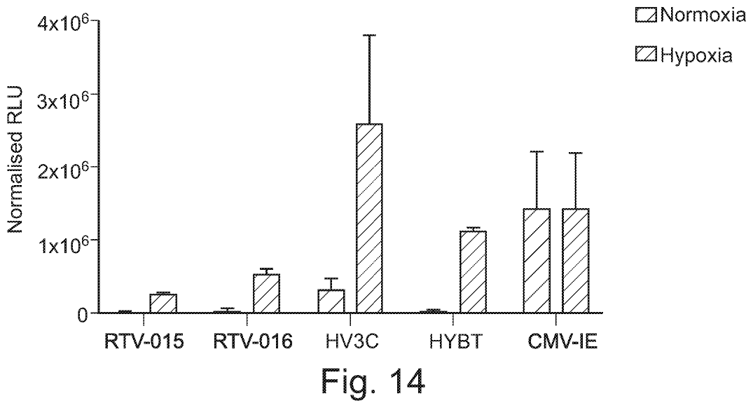

After transfection the cells were incubated for 24 hrs in normoxic conditions (20% oxygen). Induction of the promoters was assessed by using luciferase activity after 24 hrs in normoxia or hypoxia (5% oxygen, 10% carbon dioxide and 85% nitrogen). Hypoxia was achieved by gas displacement in a sealed hypoxia chamber. Results are shown in FIG. 14.

### Measurement of Luciferase Activity

Luciferase activity was measured using LARII (Dual Luciferase Reporter 1000 assay system, Promega, E1980).

Media was removed from the cells at the respective time point (0, 3, 5, 24 hrs after induction). The cells were washed once in 300 μl of DPBS. Cells were lysed by adding 100 μl of passive lysis buffer to the cells and incubation with rocking for 15 minutes. The cell debris was pelleted by centrifugation of the plate at max speed in a benchtop centrifuge for 1 min. 10 μl of supernatant was pipetted into white 96-well plate and luminescence measured by addition of 50 μl of LARII substrate.

β-galactosidase activity was measured as per manufacturer's instructions (Mammalian βGalactosidase Assay Kit, 75707/75710, Thermo Scientific) using 25 μl of lysate. 25 μl of lysate was transferred into a microplate well and mixed with 25 μl of β-galactosidase Assay Reagent, equilibrated to room temperature. The mixture was incubated at 37° C. for 30 min and absorbance measured at 405 nm.

Luciferase readouts were normalised to β-galactosidase to produce normalised relative luminometer units (RLUs).

### Results

The described promoters were transiently transfected into either the suspension cell line HEK293-F (FIG. 13) or the adherent HEK293-T (FIG. 14) cell line and activity of the promoters was assessed using luciferase assay.

In FIG. 13, all of the promoters in HEK293-F cells showed a rapid increase in activity upon a switch to hypoxic conditions with an increase in luciferase activity observed after 3 hrs. Maximal activity was observed after 5 hrs for all of the promoters tested with no significant increase in activity at the 24 hr timepoint. The promoter's activities correlate with their designs, with RTV-015 being the weakest, followed by SYNP-HYP-001, RTV-016, HYBT and HV3C being the strongest. The activities of these promoters give a 13-fold dynamic range of activity with fold inductions approaching 1,000-fold. In contrast, the switch to hypoxia has no effect on the activity of the CMV-IE promoter and luciferase activity does not change. SYNP-HYP-001 has not been tested in the other examples.

In FIG. 14, the promoter's expression after 24 h in hypoxia was compared to their expression after 24 h in normoxia. In HEK293-T cells the pattern of expression is very similar to HEK293-F cells with RTV-015 being the weakest promoter and HV3C being the strongest with a 9-fold dynamic range observed across the promoters. Fold induction in this cell line is less dramatic with maximal induction of 50-fold observed. Again, there is no change in the CMV-IE promoter between normoxic and hypoxic conditions.

These results seem to validate our design principals with the strength of the promoters correlating to their theoretical relative strength.

### Example 6

### Transient Transfection of CHO-GS Cells

40 ml of cells were grown in a 250 ml vented Erlenmeyer flask (Sigma-Aldrich CLS431144) at 37° C., 20% O₂, 8% CO₂ with agitation at 100 rpm. Cells were seeded as at 300,000 cells/ml.

One day before transfection, the cells were counted using a haemocytometer and split to 500,000 cells/ml.

On the day of transfection, the cells are seeded to 1,000,000 cells/ml in 500 μl of appropriate medium (Thermofisher, CD-CHO 10743029) in a 24 well plate. 0.625 pg of DNA per well was then added to 10 μl of OptiMem medium (Thermofisher; 11058021) and incubated for 5 minutes at room temperature.

Concurrently, 0.625 μl of Freestyle Max reagent (Thermofisher, 16447100) was made up to 10 μl by addition of OptiMem and incubated for 5 minutes at room temperature. After this incubation, both mixes were added to the same tube and incubated at room temperature for 25-30 minutes. The DNA/Max reagent mix (20 μl/well) was then added directly to the cells and the cells incubated at 37 C, 8% CO₂ with agitation at 100 rpm.

The transfected DNA was one of the vectors where the promoter constructs (RTV-015, HYBT, RTV-016, HV3C) or a control promoter (CMV-IE) were used to drive luciferase expression and β-galactosidase containing pcDNA6 plasmid. The β-galactosidase containing plasmid was used as internal control for transfection efficiency (Thermofisher, V22020).

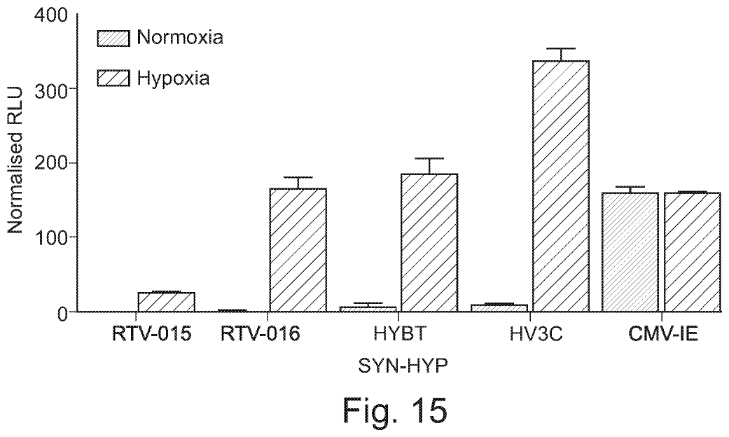

After transfection the cells were incubated for 24 hrs in normoxic conditions (20% oxygen). Induction of the promoters was assessed by using luciferase activity after 24 hrs in normoxia or hypoxia (5% oxygen, 10% carbon dioxide and 85% nitrogen). Hypoxia was achieved by gas displacement in a sealed hypoxia chamber. Luciferase activity was measured as described above. Results are shown in FIG. 15.

### Results

Luciferase expression form the promoters RTV-015, RTV-016, HV3C and HYBT was assessed in the transiently transfected CHO suspension line CHO-GSK1SV in order to test their functionality in an industrially relevant CHO strain.

As can be seen from FIG. 15, the promoters behave in a similar manner in these cells as they do in HEK293 cells. The relative strength of each promoter is proportional to the HEK293 cell results with RTV-015 being the weakest and HV3C being the strongest. The dynamic range of the promoters is 13-fold with maximal induction observed at >150 fold. Again, the switch to hypoxia has no effect on the activity of the CMV-IE promoter and luciferase activity from this promoter does not differ in hypoxia or normoxia.

This demonstrates the robustness of the promoters across multiple cell lines and validates our design rules.

### Example 7

### Generating CHO-GS-KSV1 Stable Cell Lines

### Materials

CD-CHO media (Life technologies, CAT #10743029)

Corning 125 mL Polycarbonate Erlenmeyer Flask with Vent Cap (CAT #734-1885).

Gene Pulser® Electroporation Cuvettes, 0.4 cm gap (BioRad, CAT #165-2088)

Gene Pulser Xcell Total System (BioRad, CAT, #1652660)

GS-vector DNA (40 pg in 100 μL TE buffer) linearised with Sca1

Suspension cultures of CHOK1SV GS-KO host cells.

Cell suspensions of 6×10⁵ cells per mL are incubated on an orbital shaker set at 8% CO₂, 20% O₂, 37° C., 85% relative humidity and 140 rpm overnight. 2.86×10⁷ cells were centrifuged at 200 g for 3 minutes. Media is then aspirated and cell pellet resuspended in 2 mL fresh CD-CHO media to obtain a concentration of 1.43×10⁷ cells per mL. 700 μL of cell suspension was added to each of two electroporation cuvettes each containing 40 g of linearised DNA in 100 μL of sterile TE buffer (Thermofisher, 12090015). Each cuvette was electroporated, delivering a single pulse of 300V, 900 pF with resistance to infinity. Immediately after delivery of pulse electroplated cells were transferred to Erlenmeyer 125 mL flask containing 20 mL of CD-CHO media pre-warmed to 37° C. Electroporated cells from two cuvettes are combined into a single 125 mL flask to generate one pool of cells. Cells are cultured on an orbital shaker set at 8% CO₂, 20% 02, 37° C., 85% relative humidity and 140 rpm. Cells are transferred to fresh CD-CHO media 24 hours after transfection and cell cultures monitored and given fresh CD-CHO media every 2-4 days. Usually after about 10-14 days cell numbers will be high enough to start passaging. Cells were selected at 14 days and then expanded. Induction was performed once doubling time had returned to approx. 24 hrs. The cells assayed in this example were at passage number 15, 17, 19 and 21.

The transfected DNA was pXC-17.4 expression vector (Lonza Biologics plc) where one of the promoter constructs (RTV-015, HYBT, RTV-016, HV3C) or a control promoter (CMV-IE) have been cloned upstream of the SEAP gene, which had been cloned into the multiple cloning site within the vector expression cassette. Promoters were closed into the pXC-17.4 vector using Gibson assembly. The pXC-17.4 expression vector is designed for making stable cell lines in the CHO-GSK1SV cell line as it contains the glutamate synthase gene which has been knocked out of the cell line. Therefore, selection of cells in glutamine drop-out medium will select for cells that have stably integrated the plasmid.

Stably transfected CHO-GS cells were seeded at 500,000 cells/ml and allowed to grow for 24 hrs, at this point the cells were counted and placed under hypoxic conditions as previously described. After 24 hrs, the cell numbers and the expression of SEAP was measured.

### SEAP Assay

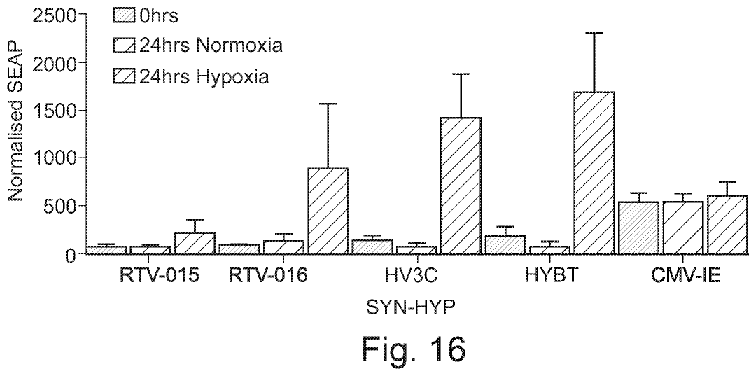

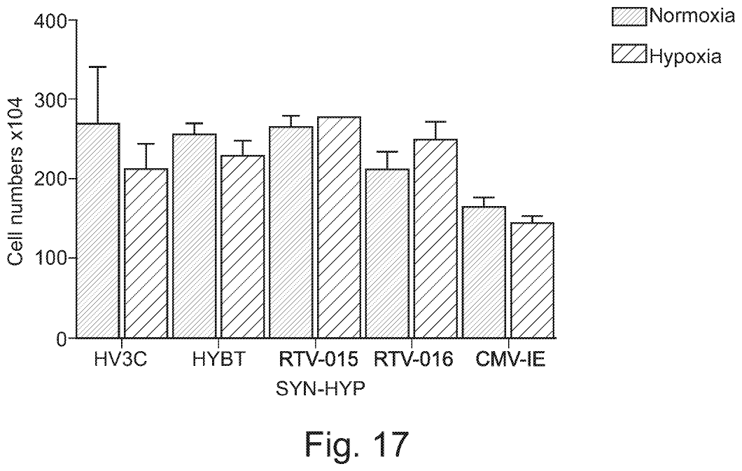

SEAP Reporter Gene Assay, chemiluminescent (Roche, CAT #11 779 842 001) was used to measured SEAP activity as per manufacturers protocol. All reagents and samples were fully pre-equilibrated at room temperature. Culture supernatant was collected from the stably transfected CHO-GS at the specific time points (0 h and 24 h). The supernatant was diluted 1:4 in dilution buffer and heat treated at 65° C. for 30 minutes. The heat-treated sample was then centrifuged for 30 s at maximum speed. 50 μl of the heat-treated sample was then added to 5 μl of inactivation buffer and incubated for 5 min at room temperature. 50 μl of substrate reagent was then added and incubated for 10 min at room temperature. The signal is then read at 477 nm and compared to a calibration curve. SEAP expression was normalized against cell number to ensure that the increase in activity was not due to increased cell numbers and was true induction. The result of this experiment can be seen in FIG. 16 and FIG. 17.

### Results

To be a useful tool to produce proteins in a manufacturing situation the promoters must function after stable integration into the target cells. To test this the promoters were cloned upstream of the SEAP gene in the vector pXC-17.4 in CHO-GS cells and these cells were assessed for induction by hypoxia.

The activity of SEAP at 0 hrs, 24 hrs in Hypoxia and at 24 hrs of normoxia is shown in FIG. 6. This graph shows that the promoters are induced by the hypoxic conditions with relative expression levels following a similar trend to the transient transfection assays. In stable cell lines, there is a 10-fold dynamic range of the promoters with a maximal fold induction of 20. FIG. 17 shows that there is no difference in the growth of the cells in the different conditions confirming that the activity observed is due to induction of the promoters and not cell growth.

The promoter's activity in the stable cell line validates the design principles and shows that their use in biomanufacturing is feasible.

Sequences
Synp-RTV-015
(SEQ ID NO: 81)
ACGTGCGATGATGCGTAGCTAGTAGTGATGATGCGTAGCTAGTAGTACGTGCGATGATGCGTAG
CTAGTAGTGATGATGCGTAGCTAGTAGTACGTGCGATGATGCGTAGCTAGTAGTGATGATGCGT
AGCTAGTAGTACGTGCGATGATGCGTAGCTAGTAGTGATGATGCGTAGCTAGTAGTACGTGCTT
GGTACCATCCGGGCCGGCCGCTTAAGCGACGCCTATAAAAAATAGGTTGCATGCTAGGCCTA
GCGCTGCCAGTCCATCTTCGCTAGCCTGTGCTGCGTCAGTCCAGCGCTGCGCTGCGTAACGGC
CGCC
HRE1 underlined, MP1 minimal promoter in bold.
Synp-RTV-016
(SEQ ID NO: 82)
CTGCACGTAGATGATGCGTAGCTAGTAGTCTGCACGTAGATGATGCGTAGCTAGTAGTCTGCAC
GTAGATGATGCGTAGCTAGTAGTCTGCACGTAGATGATGCGTAGCTAGTAGTCTGCACGTAGAT
GATGCGTAGCTAGTAGTCTGCACGTAGTAGTCGTATGCTGATGCGCAGTTAGCGTAGCTGAGGT
ACCGTCGACGATATCGGATCCAGGTCTATATAAGCAGAGCTCGTTTAGTGAACCGTCAGATCG
CCTAGATACGCCATCCACGCTGTTTTGACCTCCATAGAAGATCGCCACC
HRE2 underlined, CMV-MP minimal promoter bold.
Synp-HYBT
(SEQ ID NO: 86)
GATCTTTGTATTTAATTAAGACCTTGAGTACGTGCGTCTCTGCACGTATGGCGATTAAGACCTTGA
GTACGTGCGTCTCTGCACGTATGGCGATTAAGACCTTGAGTACGTGCGTCTCTGCACGTATGGC
GATTAAGACCTTGAGTACGTGCGTCTCTGCACGTATGGCGATTAATCCATATGCTCTAGAGGGTA
TATAATGGGGGCCACTAGTCTACTACCAGAAAGCTTGGTACCGAGCTCGGATCCAGCCACC
HRE3 underlined, YB-TATA minimal promoter bold
Synp-HV3C
(SEQ ID NO: 87)
GATCTTTGTATTTAATTAAGACCTTGAGTACGTGCGTCTCTGCACGTATGGCGATTAAGACCTTGA
GTACGTGCGTCTCTGCACGTATGGCGATTAAGACCTTGAGTACGTGCGTCTCTGCACGTATGGC
GATTAAGACCTTGAGTACGTGCGTCTCTGCACGTATGGCGATTAATCCATATGCAGGTCTATATA
AGCAGAGCTCGTTTAGTGAACCGTCAGATCGCCTAGATACGCCATCCACGCTGTTTTGACCTC
CATAGAAGATCGCCACC
HRE3 underlined, CMV-MP minimal promoter bold
Synp-HYP-001
(SEQ ID NO: 83)
CTGCACGTACTGCACGTACTGCACGTACTGCACGTATGGGTACCGTCGACGATATCGGATCCAG
GTCTATATAAGCAGAGCTCGTTTAGTGAACCGTCAGATCGCCTAGATACGCCATCCACGCTG
TTTTGACCTCCATAGAAGATCGCCACC
HRE2 underlined, minimal promoter bold
pMA-RQ luciferase vector - RTV-015
(SEQ ID NO: 88)
ACGTGCGATGAGCTCCCCGGGTTAATTAACATATGACTAGTGAATTCATTGATCATAATCAGCCA
TACCACATTTGTAGAGGTTTTACTTGCTTTAAAAAACCTCCCACACCTCCCCCTGAACCTGAAACA
TAAAATGAATGCAATTGTTGTTGTTAACTTGTTTATTGCAGCTTATAATGGTTACAAATAAAGCAAT
AGCATCACAAATTTCACAAATAAAGCATTTTTTTCACTGCATTCTAGTTGTGGTTTGTCCAAACTCA
TCAATGTATCTTATCATGTCTGGCGGCCGCGACCTGCAGGCGCAGAACTGGTAGGTATGGAAGA
TCCCTCGAGATCCATTGTGCTGGCGGTAGGCGAGCAGCGCCTGCCTGAAGCTGCGGGCATTCC
CGATCAGAAATGAGCGCCAGTCGTCGTCGGCTCTCGGCACCGAATGCGTATGATTCTCCGCCAG
CATGGCTTCGGCCAGTGCGTCGAGCAGCGCCCGCTTGTTCCTGAAGTGCCAGTAAAGCGCCGG
CTGCTGAACCCCCAACCGTTCCGCCAGTTTGCGTGTCGTCAGACCGTCTACGCCGACCTCGTTC
AACAGGTCCAGGGCGGCACGGATCACTGTATTCGGCTGCAACTTTGTCATGCTTGACACTTTATC
ACTGATAAACATAATATGTCCACCAACTTATCAGTGATAAAGAATCCGCGCCAGCACAATGGATC
TCGAGGTCGAGGGATCTCTAGAGGATCCTCTACGCCGGACGCATCGTGGCCGGCATCACCGGC
GCCACAGGTGCGGTTGCTGGCGCCTATATCGCCGACATCACCGATGGGGAAGATCGGGCTCGC
CACTTCGGGCTCATGAGCGCTTGTTTCGGCGTGGGTATGGTGGCAGGCCCCGTGGCCGGGGGA
CTGTTGGGCGCCATCTCCTTGCATGCACCATTCCTTGCGGCGGCGGTGCTCAACGGCCTCAACC
TACTACTGGGCTGCTTCCTAATGCAGGAGTCGCATAAGGGAGAGCGTCGACCTCGGGCCGCGT
TGCTGGCGTTTTTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAAGCTCCCTCGTG
CGCTCTCCTGTTCCGACCCTGCCGCTTACCGGATACCTGTCCGCCTTTCTCCCTTCGGGAAGCG
TGGCGCTTTCTCATAGCTCACGCTGTAGGTATCTCAGTTCGGTGTAGGTCGTTCGCTCCAAGCTG
GGCTGTGTGCACGAACCCCCCGTTCAGCCCGACCGCTGCGCCTTATCCGGTAACTATCGTCTTG
AGTCCAACCCGGTAAGACACGACTTATCGCCACTGGCAGCAGCCACTGGTAACAGGATTAGCAG
AGCGAGGTATGTAGGCGGTGCTACAGAGTTCTTGAAGTGGTGGCCTAACTACGGCTACACTAGA
AGAACAGTATTTGGTATCTGCGCTCTGCTGAAGCCAGTTACCTTCGGAAAAAGAGTTGGTAGCTC
TTGATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGGAAC
GAAAACTCACGTTAAGGGATTTTGGTCATGAGATTATCAAAAAGGATCTTCACCTAGATCCTTTTA
AATTAAAAATGAAGTTTTAAATCAATCTAAAGTATATATGAGTAAACTTGGTCTGACAGTTACCAAT
GCTTAATCAGTGAGGCACCTATCTCAGCGATCTGTCTATTTCGTTCATCCATAGTTGCCTGACTC
CCCGTCGTGTAGATAACTACGATACGGGAGGGCTTACCATCTGGCCCCAGTGCTGCAATGATAC
CGCGAGACCCACGCTCACCGGCTCCAGATTTATCAGCAATAAACCAGCCAGCCGGAAGGGCCG
AGCGCAGAAGTGGTCCTGCAACTTTATCCGCCTCCATCCAGTCTATTAATTGTTGCCGGGAAGCT
AGAGTAAGTAGTTCGCCAGTTAATAGTTTGCGCAACGTTGTTGCCATTGCTACAGGCATCGTGGT
GTCACGCTCGTCGTTTGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATCGTTGTCAGAAGTAAG
TTGGCCGCAGTGTTATCACTCATGGTTATGGCAGCACTGCATAATTCTCTTACTGTCATGCCATC
CGTAAGATGCTTTTCTGTGACTGGTGAGTACTCAACCAAGTCATTCTGAGAATAGTGTATGCGGC
GACCGAGTTGCTCTTGCCCGGCGTCAATACGGGATAATACCGCGCCACATAGCAGAACTTTAAA
AGTGCTCATCATTGGAAAACGTTCTTCGGGGCGAAAACTCTCAAGGATCTTACCGCTGTTGAGAT
CCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAAT
GTTGAATACTCATACTCTTCCTTTTTCAATATTATTGAAGCATTTATCAGGGTTATTGTCTCATGAG
CGGATACATATTTGAATGTATTTAGAAAAATAAACAAATAGGGGTTCCGCGCACATTTCCCCGAAA
AGTGCCACCTGACGTCTAAGAAACCATTATTATCATGACATTAACCTATAAAAATAGGCGTATCAC
GAGGCCCTGATGGCTCTTTGCGGCACCCATCGTTCGTAATGTTCCGTGGCACCGAGGACAACCC
TCAAGAGAAAATGTAATCACACTGGCTCACCTTCGGGTGGGCCTTTCTGCGTTTATAAGGAGACA
CTTTATGTTTAAGAAGGTTGGTAAATTCCTTGCGGCTTTGGCAGCCAAGCTAGATCCGGCTGTGG
AATGTGTGTCAGTTAGGGTGTGGAAAGTCCCCAGGCTCCCCAGCAGGCAGAAGTATGCAAAGCA
TGCATCTCAATTAGTCAGCAACCAGGTGTGGAAAGTCCCCAGGCTCCCCAGCAGGCAGAAGTAT
GCAAAGCATGCATCTCAATTAGTCAGCAACCATAGTCCCGCCCCTAACTCCGCCCATCCCGCCC
CTAACTCCGCCCAGTTCCGCCCATTCTCCGCCCCATGGCTGACTAATTTTTTTTATTTATGCAGAG
GCCGAGGCCGCCTCGGCCTCTGAGCTATTCCAGAAGTAGTGAGGAGGCTTTTTTGGAGGCCTA
GGCTTTTGCAAAAAGCTAGCTTGGGGCCACCGCTCAGAGCACCTTCCACCATGGCCACCTCAGC
AAGTTCCCACTTGAACAAAAACATCAAGCAAATGTACTTGTGCCTGCCCCAGGGTGAGAAAGTCC
AAGCCATGTATATCTGGGTTGATGGTACTGGAGAAGGACTGCGCTGCAAAACCCGCACCCTGGA
CTGTGAGCCCAAGTGTGTAGAAGAGTTACCTGAGTGGAATTTTGATGGCTCTAGTACCTTTCAGT
CTGAGGGCTCCAACAGTGACATGTATCTCAGCCCTGTTGCCATGTTTCGGGACCCCTTCCGCAG
AGATCCCAACAAGCTGGTGTTCTGTGAAGTTTTCAAGTACAACCGGAAGCCTGCAGAGACCAATT
TAAGGCACTCGTGTAAACGGATAATGGACATGGTGAGCAACCAGCACCCCTGGTTTGGAATGGA
ACAGGAGTATACTCTGATGGGAACAGATGGGCACCCTTTTGGTTGGCCTTCCAATGGCTTTCCTG
GGCCCCAAGGTCCGTATTACTGTGGTGTGGGCGCAGACAAAGCCTATGGCAGGGATATCGTGG
AGGCTCACTACCGCGCCTGCTTGTATGCTGGGGTCAAGATTACAGGAACAAATGCTGAGGTCAT
GCCTGCCCAGTGGGAGTTCCAAATAGGACCCTGTGAAGGAATCCGCATGGGAGATCATCTCTGG
GTGGCCCGTTTCATCTTGCATCGAGTATGTGAAGACTTTGGGGTAATAGCAACCTTTGACCCCAA
GCCCATTCCTGGGAACTGGAATGGTGCAGGCTGCCATACCAACTTTAGCACCAAGGCCATGCGG
GAGGAGAATGGTCTGAAGCACATCGAGGAGGCCATCGAGAAACTAAGCAAGCGGCACCGGTAC
CACATTCGAGCCTACGATCCCAAGGGGGGCCTGGACAATGCCCGTCGTCTGACTGGGTTCCAC
GAAACGTCCAACATCAACGACTTTTCTGCTGGTGTCGCCAATCGCAGTGCCAGCATCCGCATTC
CCCGGACTGTCGGCCAGGAGAAGAAAGGTTACTTTGAAGACCGCCGCCCCTCTGCCAATTGTGA
CCCCTTTGCAGTGACAGAAGCCATCGTCCGCACATGCCTTCTCAATGAGACTGGCGACGAGCCC
TTCCAATACAAAAACTAATTAGACTTTGAGTGATCTTGAGCCTTTCCTAGTTCATCCCACCCCGCC
CCAGAGAGATCTTTGTGAAGGAACCTTACTTCTGTGGTGTGACATAATTGGACAAACTACCTACA
GAGATTTAAAGCTCTAAGGTAAATATAAAATTTTTAAGTGTATAATGTGTTAAACTACTGATTCTAA
TTGTTTGTGTATTTTAGATTCCAACCTATGGAACTGATGAATGGGAGCAGTGGTGGAATGCCTTTA
ATGAGGAAAACCTGTTTTGCTCAGAAGAAATGCCATCTAGTGATGATGAGGCTACTGCTGACTCT
CAACATTCTACTCCTCCAAAAAAGAAGAGAAAGGTAGAAGACCCCAAGGACTTTCCTTCAGAATT
GCTAAGTTTTTTGAGTCATGCTGTGTTTAGTAATAGAACTCTTGCTTGCTTTGCTATTTACACCACA
AAGGAAAAAGCTGCACTGCTATACAAGAAAATTATGGAAAAATATTCTGTAACCTTTATAAGTAGG
CATAACAGTTATAATCATAACATACTGTTTTTTCTTACTCCACACAGGCATAGAGTGTCTGCTATTA
ATAACTATGCTCAAAAATTGTGTACCTTTAGCTTTTTAATTTGTAAAGGGGTTAATAAGGAATATTT
GATGTATAGTGCCTTGACTAGAGATCATAATCAGCCATACCACATTTGTAGAGGTTTTACTTGCTT
TAAAAAACCTCCCACACCTCCCCCTGAACCTGAAACATAAAATGAATGCAATTGTTGTTGTTAACT
TGTTTATTGCAGCTTATAATGGTTACAAATAAAGCAATAGCATCACAAATTTCACAAATAAAGCATT
TTTTTCACTGCATTCTAGTTGTGGTTTGTCCAAACTCATCAATGTATCTTATCATGTCTGGATCTCT
AGCTTCGTGTCAAGGACGGTGAGG
pMA-RQ luciferase vector - RTV-016
(SEQ ID NO: 89)
TAATACGACTCACTATAGGGCGAATTGGCGGAAGGCCGTCAAGGCCGCATGCATAATAAAATAT
CTTTATTTTCATTACATCTGTGTGTTGGTTTTTTGTGTGCTGCACGTAGATGATGCGTAGCTAGTA
GTCTGCACGTAAGATGATGCGTAGCTAGTAGTCTGCACGTAGATGATGCGTAGCTAGTAGTCTG
CACGTAGATGATGCGTAGCTAGTAGTCTGCACGTAGATGATGCGTAGCTAGTAGTCTGCACGTA
GTAGTCGTATGCTGATGCGCAGTTAGCGTAGCTGAGGTACCGTCGACGATATCGGATCCAGGTC
TATATAAGCAGAGCTCGTTTAGTGAACCGTCAGATCGCCTAGATACGCCATCCACGCTGTTTTGA
CCTCCATAGAAGATCGCCACCATGGAAGATGCCAAAAACATTAAGAAGGGCCCAGCGCCATTCT
ACCCACTCGAAGACGGGACCGCCGGCGAGCAGCTGCACAAAGCCATGAAGCGCTACGCCCTGG
TGCCCGGCACCATCGCCTTTACCGACGCACATATCGAGGTGGACATTACCTACGCCGAGTACTT
CGAGATGAGCGTTCGGCTGGCAGAAGCTATGAAGCGCTATGGGCTGAATACAAACCATCGGATC
GTGGTGTGCAGCGAGAATAGCTTGCAGTTCTTCATGCCCGTGTTGGGTGCCCTGTTCATCGGTG
TGGCTGTGGCCCCAGCTAACGACATCTACAACGAGCGCGAGCTGCTGAACAGCATGGGCATCA
GCCAGCCCACCGTCGTATTCGTGAGCAAGAAAGGGCTGCAAAAGATCCTCAACGTGCAAAAGAA
GCTACCGATCATACAAAAGATCATCATCATGGATAGCAAGACCGACTACCAGGGCTTCCAAAGCA
TGTACACCTTCGTGACTTCCCATTTGCCACCCGGCTTCAACGAGTACGACTTCGTGCCCGAGAG
CTTCGACCGGGACAAAACCATCGCCCTGATCATGAACAGTAGTGGCAGTACCGGATTGCCCAAG
GGCGTAGCCCTACCGCACCGCACCGCTTGTGTCCGATTCAGTCATGCCCGCGACCCCATCTTCG
GCAACCAGATCATCCCCGACACCGCTATCCTCAGCGTGGTGCCATTTCACCACGGCTTCGGCAT
GTTCACCACGCTGGGCTACTTGATCTGCGGCTTTCGGGTCGTGCTCATGTACCGCTTCGAGGAG
GAGCTATTCTTGCGCAGCTTGCAAGACTATAAGATTCAATCTGCCCTGCTGGTGCCCACACTATT
TAGCTTCTTCGCTAAGAGCACTCTCATCGACAAGTACGACCTAAGCAACTTGCACGAGATCGCCA
GCGGCGGGGCGCCGCTCAGCAAGGAGGTAGGTGAGGCCGTGGCCAAACGCTTCCACCTACCA
GGCATCCGCCAGGGCTACGGCCTGACAGAAACAACCAGCGCCATTCTGATCACCCCCGAAGGG
GACGACAAGCCTGGCGCAGTAGGCAAGGTGGTGCCCTTCTTCGAGGCTAAGGTGGTGGACTTG
GACACCGGTAAGACACTGGGTGTGAACCAGCGCGGCGAGCTGTGCGTCCGTGGCCCCATGATC
ATGAGCGGCTACGTTAACAACCCCGAGGCTACAAACGCTCTCATCGACAAGGACGGCTGGCTGC
ACAGCGGCGACATCGCCTACTGGGACGAGGACGAGCACTTCTTCATCGTGGACCGGCTGAAGA
GCCTGATCAAATACAAGGGCTACCAGGTAGCCCCAGCCGAACTGGAGAGCATCCTGCTGCAACA
CCCCAACATCTTCGACGCCGGGGTCGCCGGCCTGCCCGACGACGATGCCGGCGAGCTGCCCG
CCGCAGTCGTCGTGCTGGAACACGGTAAAACCATGACCGAGAAGGAGATCGTGGACTATGTGG
CCAGCCAGGTTACAACCGCCAAGAAGCTGCGCGGTGGTGTTGTGTTCGTGGACGAGGTGCCTA
AAGGACTGACCGGCAAGTTGGACGCCCGCAAGATCCGCGAGATTCTCATTAAGGCCAAGAAGG
GCGGCAAGATCGCCGTGTAATGAAAGCTTGGTCTCTACGAGTAATAGACGCCCAGTTGAATTCC
TTCGAGCAGACATGATAAGATACATTGATGAGTTTGGACAAACCACAACTAGAATGCAGTGAAAA
AAATGCTTTATTTGTGAAATTTGTGATGCTATTGCTTTATTTGTAACCATTATAAGCTGCAATAAAC
AAGTTAACAACAACAATTGCATTCATTTTATGTTTCAGGTTCAGGGGGAGGTGTGGGAGGTTTTTT
AAAGCAAGTAAAACCTCTACAAATGTGGTAAAATCGATAAGGATCCGTCTGGGCCTCATGGGCCT
TCCGCTCACTGCCCGCTTTCCAGTCGGGAAACCTGACAACAACAATTGCATTCATTTTATGTTTCA
GGTTCAGGGGGAGGTGTGGGAGGTTTTTTAAAGCAAGTAAAACCTCTACAAATGTGGTAAAATCG
ATAAGGATCCGTCTGGGCCTCATGGGCCTTCCGCTCACTGCCCGCTTTCCAGTCGGGAAACCTG
TCGTGCCAGCTGCATTAACATGGTCATAGCTGTTTCCTTGCGTATTGGGCGCTCTCCGCTTCCTC
GCTCACTGACTCGCTGCGCTCGGTCGTTCGGGTAAAGCCTGGGGTGCCTAATGAGCAAAAGGC
CAGCAAAAGGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGACCCTGCCGCTTACC
GGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTACAGAG
TTCTTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTGGT
AGCGGTGGTTTTTTTGTTTGCAAGCAGCAGATTACGCGCAGAAAAAAAGGATCTCAAGAAGATCC
TTTGATCTTTTCTACGGGGTCTGACGCTCAGTGGAACGAAAACTCACGTTAAGGGATTTTGGTCA
TGAGATTATCAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCTATCTCAGC
GATCTGTCTATTTCGTTCATCCATAGTTGCCTGACTCCCCGTCGTGTAGATAACTACGATACGGG
AGGGCTTACCATCTGGCCCCAGTGCTGCAATGATACCGCGAGAACCACGCTCACCGGCTCCAG
ATTTATCAGCAATAAACCAGCCAGCCGGAAGGGCCGAGCGCAGAAGTGGTCCTGCAACTTTATC
CGCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTC
ATTCAGCTCCGGTTCCCAACGATCAAGGCGAGTTACATGATCCCCCATGTTGTGCAAAAAAGCG
GTTAGCTCCTTCGGTCCTCCGATCGTTGTCAGAAGTAAGTTGGCCGCAGTGTTATCACTCATGGT
TATGGCAGCACTGCATAATTCTCTTACTGTCATGCCATCCGTAAGATGCTTTTCTGTGACTGGTGA
GTACTCAACCAAGTCATTCTGAGAATAGTGTATGCGGCGACCGAGTTGCTCTTGCCCGGCGTCA
ATACGGGATAATACCGCGCCACATAGCAGAACTTTAAAAGTGCTCATCATTGGAAAACGTTCTTC
GGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCA
AAATGCCGCAAAAAAGGGAATAAGGGCGACACGGAAATGTTGAATACTCATACTCTTCCTTTTTC
AATATTATTGAAGCATTTATCAGGGTTATTGTCTCATGAGCGGATACATATTTGAATGTATTTAGAA
AAATAAACAAATAGGGGTTCCGCGCACATTTCCCCGAAAAGTGCCAC
pMA-RQ luciferase vector - HYBT
(SEQ ID NO: 90)
CTAAATTGTAAGCGTTAATATTTTGTTAAAATTCGCGTTAAATTTTTGTTAAATCAGCTCATTTTTTA
ACCAATAGGCCGAAATCGGCAAAATCCCTTATAAATCAAAAGAATAGACCGAGATAGGGTTGAGT
GGCCGCTACAGGGCGCTCCCATTCGCCATTCAGGCTGCGCAACTGTTGGGAAGGGCGTTTCGG
TGCGGGCCTCTTCGCTATTACGCCAGCTGGCGAAAGGGGGATGTGCTGCAAGGCGATTAAGTT
GGGTAACGCCAGGGTTTTCCCAGTCACGACGTTGTAAAACGACGGCCAGTGAGCGCGACGTAAT
ACGACTCACTATAGGGCGAATTGGCGGAAGGCCGTCAAGGCCGCATGCATAATAAAATATCTTTA
TTTTCATTACATCTGTGTGTTGGTTTTTTGTGTGGACGGATCGGGAGATCTTTGTATTTAATTAAG
ACCTTGAGTACGTGCGTCTCTGCACGTATGGCGATTAAGACCTTGAGTACGTGCGTCTCTGCAC
GTATGGCGATTAAGACCTTGAGTACGTGCGTCTCTGCACGTATGGCGATTAAGACCTTGAGTAC
GTGCGTCTCTGCACGTATGGCGATTAATCCATATGCTCTAGAGGGTATATAATGGGGGCCACTA
GTCTACTACCAGAAAGCTTGGTACCGAGCTCGGATCCAGCCACCATGGAAGATGCCAAAAACAT
TAAGAAGGGCCCAGCGCCATTCTACCCACTCGAAGACGGGACCGCCGGCGAGCAGCTGCACAA
AGCCATGAAGCGCTACGCCCTGGTGCCCGGCACCATCGCCTTTACCGACGCACATATCGAGGT
GGACATTACCTACGCCGAGTACTTCGAGATGAGCGTTCGGCTGGCAGAAGCTATGAAGCGCTAT
GGGCTGAATACAAACCATCGGATCGTGGTGTGCAGCGAGAATAGCTTGCAGTTCTTCATGCCCG
TGTTGGGTGCCCTGTTCATCGGTGTGGCTGTGGCCCCAGCTAACGACATCTACAACGAGCGCGA
GCTGCTGAACAGCATGGGCATCAGCCAGCCCACCGTCGTATTCGTGAGCAAGAAAGGGCTGCA
AAAGATCCTCAACGTGCAAAAGAAGCTACCGATCATACAAAAGATCATCATCATGGATAGCAAGA
CCGACTACCAGGGCTTCCAAAGCATGTACACCTTCGTGACTTCCCATTTGCCACCCGGCTTCAAC
GAGTACGACTTCGTGCCCGAGAGCTTCGACCGGGACAAAACCATCGCCCTGATCATGAACAGTA
GTGGCAGTACCGGATTGCCCAAGGGCGTAGCCCTACCGCACCGCACCGCTTGTGTCCGATTCA
GTCATGCCCGCGACCCCATCTTCGGCAACCAGATCATCCCCGACACCGCTATCCTCAGCGTGGT
GCCATTTCACCACGGCTTCGGCATGTTCACCACGCTGGGCTACTTGATCTGCGGCTTTCGGGTC
GTGCTCATGTACCGCTTCGAGGAGGAGCTATTCTTGCGCAGCTTGCAAGACTATAAGATTCAATC
TGCCCTGCTGGTGCCCACACTATTTAGCTTCTTCGCTAAGAGCACTCTCATCGACAAGTACGACC
TAAGCAACTTGCACGAGATCGCCAGCGGCGGGGCGCCGCTCAGCAAGGAGGTAGGTGAGGCC
GTGGCCAAACGCTTCCACCTACCAGGCATCCGCCAGGGCTACGGCCTGACAGAAACAACCAGC
GCCATTCTGATCACCCCCGAAGGGGACGACAAGCCTGGCGCAGTAGGCAAGGTGGTGCCCTTC
TTCGAGGCTAAGGTGGTGGACTTGGACACCGGTAAGACACTGGGTGTGAACCAGCGCGGCGAG
CTGTGCGTCCGTGGCCCCATGATCATGAGCGGCTACGTTAACAACCCCGAGGCTACAAACGCTC
TCATCGACAAGGACGGCTGGCTGCACAGCGGCGACATCGCCTACTGGGACGAGGACGAGCACT
TCTTCATCGTGGACCGGCTGAAGAGCCTGATCAAATACAAGGGCTACCAGGTAGCCCCAGCCGA
ACTGGAGAGCATCCTGCTGCAACACCCCAACATCTTCGACGCCGGGGTCGCCGGCCTGCCCGA
CGACGATGCCGGCGAGCTGCCCGCCGCAGTCGTCGTGCTGGAACACGGTAAAACCATGACCGA
GAAGGAGATCGTGGACTATGTGGCCAGCCAGGTTACAACCGCCAAGAAGCTGCGCGGTGGTGT
TGTGTTCGTGGACGAGGTGCCTAAAGGACTGACCGGCAAGTTGGACGCCCGCAAGATCCGCGA
GATTCTCATTAAGGCCAAGAAGGGCGGCAAGATCGCCGTGTAATGAAAGCTTGGTCTCTACGAG
TAATAGACGCCCAGTTGAATTCCTTCGAGCAGACATGATAAGATACATTGATGAGTTTGGACAAA
CCACAACTAGAATGCAGTGAAAAAAATGCTTTATTTGTGAAATTTGTGATGCTATTGCTTTATTTGT
AACCATTATAAGCTGCAATAAACAAGTTAACAACAACAATTGCATTCATTTTATGTTTCAGGTTCAG
GGGGAGGTGTGGGAGGTTTTTTAAAGCAAGTAAAACCTCTACAAATGTGGTAAAATCGATAAGGA
TCCGTCTGGGCCTCATGGGCCTTCCGCTCACTGCCCGCTTTCCAGTCGGGAAACCTGTCGTGCC
AGCTGCATTAACATGGTCATAGCTGTTTCCTTGCGTATTGGGCGCTCTCCGCTTCCTCGCTCACT
GACTCGCTGCGCTCGGTCGTTCGGGTAAAGCCTGGGGTGCCTAATGAGCAAAAGGCCAGCAAA
AGGCCAGGAACCGTAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGCCCGACC
GCTGCGCCTTATCCGGTAACTATCGTCTTGAGTCCAACCCGGTAAGACACGACTTATCGCCACT
GGCAGCAGCCACTGGTAACAGGATTAGCAGAGCGAGGTATGTAGGCGGTGCTACAGAGTTCTT
GAAGTGGTGGCCTAACTACGGCTACACTAGAAGAACAGTATTTGGTATCTGCGCTCTGCTGAAG
CCAGTTACCTTCGGAAAAAGAGTTGGTAGCTCTTGATCCGGCAAACAAACCACCGCTGGTAGCG
GTGGTTTTTTTGTTTGCAAGCAGCAGATTACGCGCAGAAAAAAAGGATCTCAAGAAGATCCTTTG
ATCTTTTCTACGGGGTCTGACGCTCAGTGGAACGAAAACTCACGTTAAGGGATTTTGGTCATGAG
ATTATCAAAAAGGATCTTCACCTAGATCCTTTTAAATTAAAAATGAAGTTTTAAATCAATCTAAAGT
ATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGG
GCTTACCATCTGGCCCCAGTGCTGCAATGATACCGCGAGAACCACGCTCACCGGCTCCAGATTT
ATCAGCAATAAACCAGCCAGCCGGAAGGGCCGAGCGCAGAAGTGGTCCTGCAACTTTATCCGC
CTCCATCCAGTCTATTAATTGTTGCCGGGAAGCTAGAGTAAGTAGTTCGCCAGTTAATAGTTTGC
GCAACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGTCAATA
CGGGATAATACCGCGCCACATAGCAGAACTTTAAAAGTGCTCATCATTGGAAAACGTTCTTCGGG
GCGAAAACTCTCAAGGATCTTACCGCTGTTGAGATCCAGTTCGATGTAACCCACTCGTGCACCCA
ACTGATCTTCAGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTCCTTTTTCAATA
TTATTGAAGCATTTATCAGGGTTATTGTCTCATGAGCGGATACATATTTGAATGTATTTAGAAAAAT
AAACAAATAGGGGTTCCGCGCACATTTCCCCGAAAAGTGCCAC
pMA-RQ luciferase vector - HV3C
(SEQ ID NO: 91)
CTAAATTGTAAGCGTTAATATTTTGTTAAAATTCGCGTTAAATTTTTGTTAAATCAGCTCATTTTTTA
ACCAATAGGCCGAAATCGGCAAAATCCCTTATAAATCAAAAGAATAGACCGAGATAGGGTTGAGT
GGCCGCTACAGGGCGCTCCCATTCGCCATTCAGGCTGCGCAACTGTTGGGAAGGGCGTTTCGG
TGCGGGCCTCTTCGCTATTACGCCAGCTGGCGAAAGGGGGATGTGCTGCAAGGCGATTAAGTT
GGGTAACGCCAGGGTTTTCCCAGTCACGACGTTGTAAAACGACGGCCAGTGAGCGCGACGTAAT
ACGACTCACTATAGGGCGAATTGGCGGAAGGCCGTCAAGGCCGCATGCATAATAAAATATCTTTA
TTTTCATTACATCTGTGTGTTGGTTTTTTGTGTGGACGGATCGGGAGATCTTTGTATTTAATTAAG
ACCTTGAGTACGTGCGTCTCTGCACGTATGGCGATTAAGACCTTGAGTACGTGCGTCTCTGCAC
GTATGGCGATTAAGACCTTGAGTACGTGCGTCTCTGCACGTATGGCGATTAAGACCTTGAGTAC
GTGCGTCTCTGCACGTATGGCGATTAATCCATATGCAGGTCTATATAAGCAGAGCTCGTTTAGTG
AACCGTCAGATCGCCTAGATACGCCATCCACGCTGTTTTGACCTCCATAGAAGATCGCCACCATG
GAAGATGCCAAAAACATTAAGAAGGGCCCAGCGCCATTCTACCCACTCGAAGACGGGACCGCC
GGCGAGCAGCTGCACAAAGCCATGAAGCGCTACGCCCTGGTGCCCGGCACCATCGCCTTTACC
GACGCACATATCGAGGTGGACATTACCTACGCCGAGTACTTCGAGATGAGCGTTCGGCTGGCAG
AAGCTATGAAGCGCTATGGGCTGAATACAAACCATCGGATCGTGGTGTGCAGCGAGAATAGCTT
GCAGTTCTTCATGCCCGTGTTGGGTGCCCTGTTCATCGGTGTGGCTGTGGCCCCAGCTAACGAC
ATCTACAACGAGCGCGAGCTGCTGAACAGCATGGGCATCAGCCAGCCCACCGTCGTATTCGTGA
GCAAGAAAGGGCTGCAAAAGATCCTCAACGTGCAAAAGAAGCTACCGATCATACAAAAGATCATC
ATCATGGATAGCAAGACCGACTACCAGGGCTTCCAAAGCATGTACACCTTCGTGACTTCCCATTT
GCCACCCGGCTTCAACGAGTACGACTTCGTGCCCGAGAGCTTCGACCGGGACAAAACCATCGC
CCTGATCATGAACAGTAGTGGCAGTACCGGATTGCCCAAGGGCGTAGCCCTACCGCACCGCAC
CGCTTGTGTCCGATTCAGTCATGCCCGCGACCCCATCTTCGGCAACCAGATCATCCCCGACACC
GCTATCCTCAGCGTGGTGCCATTTCACCACGGCTTCGGCATGTTCACCACGCTGGGCTACTTGA
TCTGCGGCTTTCGGGTCGTGCTCATGTACCGCTTCGAGGAGGAGCTATTCTTGCGCAGCTTGCA
AGACTATAAGATTCAATCTGCCCTGCTGGTGCCCACACTATTTAGCTTCTTCGCTAAGAGCACTC
TCATCGACAAGTACGACCTAAGCAACTTGCACGAGATCGCCAGCGGCGGGGCGCCGCTCAGCA
AGGAGGTAGGTGAGGCCGTGGCCAAACGCTTCCACCTACCAGGCATCCGCCAGGGCTACGGCC
TGACAGAAACAACCAGCGCCATTCTGATCACCCCCGAAGGGGACGACAAGCCTGGCGCAGTAG
GCAAGGTGGTGCCCTTCTTCGAGGCTAAGGTGGTGGACTTGGACACCGGTAAGACACTGGGTG
TGAACCAGCGCGGCGAGCTGTGCGTCCGTGGCCCCATGATCATGAGCGGCTACGTTAACAACC
CCGAGGCTACAAACGCTCTCATCGACAAGGACGGCTGGCTGCACAGCGGCGACATCGCCTACT
GGGACGAGGACGAGCACTTCTTCATCGTGGACCGGCTGAAGAGCCTGATCAAATACAAGGGCTA
CCAGGTAGCCCCAGCCGAACTGGAGAGCATCCTGCTGCAACACCCCAACATCTTCGACGCCGG
GGTCGCCGGCCTGCCCGACGACGATGCCGGCGAGCTGCCCGCCGCAGTCGTCGTGCTGGAAC
ACGGTAAAACCATGACCGAGAAGGAGATCGTGGACTATGTGGCCAGCCAGGTTACAACCGCCAA
GAAGCTGCGCGGTGGTGTTGTGTTCGTGGACGAGGTGCCTAAAGGACTGACCGGCAAGTTGGA
CGCCCGCAAGATCCGCGAGATTCTCATTAAGGCCAAGAAGGGCGGCAAGATCGCCGTGTAATG
AAAGCTTGGTCTCTACGAGTAATAGACGCCCAGTTGAATTCCTTCGAGCAGACATGATAAGATAC
ATTGATGAGTTTGGACAAACCACAACTAGAATGCAGTGAAAAAAATGCTTTATTTGTGAAATTTGT
GATGCTATTGCTTTATTTGTAACCATTATAAGCTGCAATAAACAAGTTAACAACAACAATTGCATTC
ATTTTATGTTTCAGGTTCAGGGGGAGGTGTGGGAGGTTTTTTAAAGCAAGTAAAACCTCTACAAA
TGTGGTAAAATCGATAAGGATCCGTCTGGGCCTCATGGGCCTTCCGCTCACTGCCCGCTTTCCA
GTCGGGAAACCTGTCGTGCCAGCTGCATTAACATGGTCATAGCTGTTTCCTTGCGTATTGGGCG
CTCTCCGCTTCCTCGCTCACTGACTCGCTGCGCTCGGTCGTTCGGGTAAAGCCTGGGGTGCCTA
ATGAGCAAAAGGCCAGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAGGTGGCGAAACCCG
ACAGGACTATAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTTTCTCATAG
CTCACGCTGTAGGTATCTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTTGAGTCCAACCCGGTAA
GACACGACTTATCGCCACTGGCAGCAGCCACTGGTAACAGGATTAGCAGAGCGAGGTATGTAGG
CGGTGCTACAGAGTTCTTGAAGTGGTGGCCTAACTACGGCTACACTAGAAGAACAGTATTTGGTA
TCTGCGCTCTGCTGAAGCCAGTTACCTTCGGAAAAAGAGTTGGTAGCTCTTGATCCGGCAAACAA
ACCACCGCTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACTCACGTTAA
GGGATTTTGGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCCGTCGTGTAGAT
AACTACGATACGGGAGGGCTTACCATCTGGCCCCAGTGCTGCAATGATACCGCGAGAACCACGC
TCACCGGCTCCAGATTTATCAGCAATAAACCAGCCAGCCGGAAGGGCCGAGCGCAGAAGTGGT
CCTGCAACTTTATCCGCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGGTGTCACGCTCGTCGT
TTGGTATGGCTTCATTCAGCTCCGGTTCCCAACGATCAAGGCGAGTTACATGATCCCCCATGTTG
TGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTT
CTGTGACTGGTGAGTACTCAACCAAGTCATTCTGAGAATAGTGTATGCGGCGACCGAGTTGCTCT
TGCCCGGCGTCAATACGGGATAATACCGCGCCACATAGCAGAACTTTAAAAGTGCTCATCATTG
GAAAACGTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCATA
CTCTTCCTTTTTCAATATTATTGAAGCATTTATCAGGGTTATTGTCTCATGAGCGGATACATATTTG
AATGTATTTAGAAAAATAAACAAATAGGGGTTCCGCGCACATTTCCCCGAAAAGTGCCAC
pMA-RQ luciferase vector - Synp-HYP-001
(SEQ ID NO: 92)
AATAAAATATCTTTATTTTCATTACATCTGTGTGTTGGTTTTTTGTGTGCTGCACGTACTGCACGTA
CTGCACGTACTGCACGTATGGGTACCGTCGACGATATCGGATCCAGGTCTATATAAGCAGAGCT
CGTTTAGTGAACCGTCAGATCGCCTAGATACGCCATCCACGCTGTTTTGACCTCCATAGAAGATC
GCCACCATGGAAGATGCCAAAAACATTAAGAAGGGCCCAGCGCCATTCTACCCACTCGAAGACG
GGACCGCCGGCGAGCAGCTGCACAAAGCCATGAAGCGCTACGCCCTGGTGCCCGGCACCATC
GCCTTTACCGACGCACATATCGAGGTGGACATTACCTACGCCGAGTACTTCGAGATGAGCGTTC
GGCTGGCAGAAGCTATGAAGCGCTATGGGCTGAATACAAACCATCGGATCGTGGTGTGCAGCG
AGAATAGCTTGCAGTTCTTCATGCCCGTGTTGGGTGCCCTGTTCATCGGTGTGGCTGTGGCCCC
AGCTAACGACATCTACAACGAGCGCGAGCTGCTGAACAGCATGGGCATCAGCCAGCCCACCGT
CGTATTCGTGAGCAAGAAAGGGCTGCAAAAGATCCTCAACGTGCAAAAGAAGCTACCGATCATA
CAAAAGATCATCATCATGGATAGCAAGACCGACTACCAGGGCTTCCAAAGCATGTACACCTTCGT
GACTTCCCATTTGCCACCCGGCTTCAACGAGTACGACTTCGTGCCCGAGAGCTTCGACCGGGAC
AAAACCATCGCCCTGATCATGAACAGTAGTGGCAGTACCGGATTGCCCAAGGGCGTAGCCCTAC
CGCACCGCACCGCTTGTGTCCGATTCAGTCATGCCCGCGACCCCATCTTCGGCAACCAGATCAT
CCCCGACACCGCTATCCTCAGCGTGGTGCCATTTCACCACGGCTTCGGCATGTTCACCACGCTG
GGCTACTTGATCTGCGGCTTTCGGGTCGTGCTCATGTACCGCTTCGAGGAGGAGCTATTCTTGC
GCAGCTTGCAAGACTATAAGATTCAATCTGCCCTGCTGGTGCCCACACTATTTAGCTTCTTCGCT
AAGAGCACTCTCATCGACAAGTACGACCTAAGCAACTTGCACGAGATCGCCAGCGGCGGGGCG
CCGCTCAGCAAGGAGGTAGGTGAGGCCGTGGCCAAACGCTTCCACCTACCAGGCATCCGCCAG
GGCTACGGCCTGACAGAAACAACCAGCGCCATTCTGATCACCCCCGAAGGGGACGACAAGCCT
GGCGCAGTAGGCAAGGTGGTGCCCTTCTTCGAGGCTAAGGTGGTGGACTTGGACACCGGTAAG
ACACTGGGTGTGAACCAGCGCGGCGAGCTGTGCGTCCGTGGCCCCATGATCATGAGCGGCTAC
GTTAACAACCCCGAGGCTACAAACGCTCTCATCGACAAGGACGGCTGGCTGCACAGCGGCGAC
ATCGCCTACTGGGACGAGGACGAGCACTTCTTCATCGTGGACCGGCTGAAGAGCCTGATCAAAT
ACAAGGGCTACCAGGTAGCCCCAGCCGAACTGGAGAGCATCCTGCTGCAACACCCCAACATCTT
CGACGCCGGGGTCGCCGGCCTGCCCGACGACGATGCCGGCGAGCTGCCCGCCGCAGTCGTCG
TGCTGGAACACGGTAAAACCATGACCGAGAAGGAGATCGTGGACTATGTGGCCAGCCAGGTTAC
AACCGCCAAGAAGCTGCGCGGTGGTGTTGTGTTCGTGGACGAGGTGCCTAAAGGACTGACCGG
CAAGTTGGACGCCCGCAAGATCCGCGAGATTCTCATTAAGGCCAAGAAGGGCGGCAAGATCGC
CGTGTAATGAAAGCTTGGTCTCTACGAGTAATAGACGCCCAGTTGAATTCCTTCGAGCAGACATG
ATAAGATACATTGATGAGTTTGGACAAACCACAACTAGAATGCAGTGAAAAAAATGCTTTATTTGT
GAAATTTGTGATGCTATTGCTTTATTTGTAACCATTATAAGCTGCAATAAACAAGTTAACAACAACA
ATTGCATTCATTTTATGTTTCAGGTTCAGGGGGAGGTGTGGGAGGTTTTTTAAAGCAAGTAAAAC
CTCTACAAATGTGGTAAAATCGATAAGGATCCGTCTGGGCCTCATGGGCCTTCCGCTCACTGCC
CGCTTTCCAGTCGGGAAACCTGTCGTGCCAGCTGCATTAACATGGTCATAGCTGTTTCCTTGCGT
ATTGGGCGCTCTCCGCTTCCTCGCTCACTGACTCGCTGCGCTCGGTCGTTCGGGTAAAGCCTGG
GGTGCCTAATGAGCAAAAGGCCAGCAAAAGGCCAGGAACCGTAAAAAGGCCGCGTTGCTGGCG
TTTTTCCATAGGCTCCGCCCCCCTGACGAGCATCACAAAAATCGACGCTCAAGTCAGAGGTGGC
GAAACCCGACAGGACTATAAAGATACCAGGCGTTTCCCCCTGGAAGCTCCCTCGTGCGCTCTCC
TGTTCCGACCCTGCCGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTCCAAGCTGGGCTGTG
TGCACGAACCCCCCGTTCAGCCCGACCGCTGCGCCTTATCCGGTAACTATCGTCTTGAGTCCAA
CCCGGTAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGAAGAACA
GTATTTGGTATCTGCGCTCTGCTGAAGCCAGTTACCTTCGGAAAAAGAGTTGGTAGCTCTTGATC
CGGCAAACAAACCACCGCTGGTAGCGGTGGTTTTTTTGTTTGCAAGCAGCAGATTACGCGCAGA
AAAAAAGGATCTCAAGAAGATCCTTTGATCTTTTCTACGGGGTCTGACGCTCAGTGGAACGAAAA
CTCACGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTACCAATGCTTA
ATCAGTGAGGCACCTATCTCAGCGATCTGTCTATTTCGTTCATCCATAGTTGCCTGACTCCCCGT
CGTGTAGATAACTACGATACGGGAGGGCTTACCATCTGGCCCCAGTGCTGCAATGATACCGCGA
GAACCACGCTCACCGGCTCCAGATTTATCAGCAATAAACCAGCCAGCCGGAAGGGCCGAGCGC
AGAAGTGGTCCTGCAACTTTATCCGCCTCCATCCAGTCTATTAATTGTTGCCGGGAAGCTAGAGT
AAGTAGTTCGCCAGTTAATAGTTTGCGCAACGTTGTTGCCATTGCTACAGGCATCGTGGTGTCAC
GCTCGTCGTTTGGTATGGCTTCATTCAGCTCCGGTTCCCAACGATCAAGGCGAGTTACATGATCC
CCCATGTTGTGCAAAAAAGCGGTTAGCTCCTTCGGTCCTCCGATCGTTGTCAGAAGTAAGTTGGC
CGCAGTGTTATCACTCATGGTTATGGCAGCACTGCATAATTCTCTTACTGTCATGCCATCCGTAA
GATGCTTTTCTGTGACTGGTGAGTACTCAACCAAGTCATTCTGAGAATAGTGTATGCGGCGACCG
AGTTGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCAGTT
CGATGTAACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGAAATGTTGAA
TACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCC
ACCTAAATTGTAAGCGTTAATATTTTGTTAAAATTCGCGTTAAATTTTTGTTAAATCAGCTCATTTTT
TAACCAATAGGCCGAA
Synp-FORCSV-10
(SEQ ID NO: 46)
CCATTGACGTCACGATTACCATTGACGTCACGATTACCATTGACGTCACGATTACCATTGACGTC
ACGATTACCATTGACGTCAGCGATTAAGATGACTCAGCGATTAAGATGACTCAGCGATTAAGATG
ACTCAGCGATTAAGATGACTCACTAGCCCGGGCTCGAGATCTGCGATCTGCATCTCAATTAGTC
AGCAACCATAGTCCCGCCCCTAACTCCGCCCATCCCGCCCCTAACTCCGCCCAGTTCCGCCCA
TTCTCCGCCCCATCGCTGACTAATTTTTTTTATTTATGCAGAGGCCGAGGCCGCCTCGGCCTCT
GAGCTATTCCAGAAGTAGTGAGGAGGCTTTTTTGGAGGCCTAGGCTTTTGCAAAAAGCTTGGC
ATTCCGGTACTGTTGGTAAAGCCACC
cAMPRE and AP1 underlined, minimal promoter bold
Synp-FORCMV-09
(SEQ ID NO: 47)
CCATTGACGTCACGATTACCATTGACGTCACGATTACCATTGACGTCACGATTACCATTGACGTC
ACGATTACCATTGACGTCAGCGATTAAGATGACTCAGCGATTAAGATGACTCAGCGATTAAGATG
ACTCAGCGATTAAGATGACTCAGCGATTAATCCATATGCAGGTCTATATAAGCAGAGCTCGTTTA
GTGAACCGTCAGATCGCCTAGATACGCCATCCACGCTGTTTTGACCTCCATAGAAGATCGCCA
CC
CAMPRE and AP1 underlined, minimal promoter bold
Synp-FMP-02
(SEQ ID NO: 41)
TGAGTCAGATGATGCGTAGCTAGTAGTTGAGTCAGATGATGCGTAGCTAGTAGTTGAGTCAGATG
ATGCGTAGCTAGTAGTTGAGTCAGATGATGCGTAGCTAGTAGTTGAGTCAGATGATGCGTAGCTA
GTAGTTGAGTCAGATGATGCGTAGCTAGTAGTTGAGTCAGATGATGCGTAGCTAGTAGTTGAGTC
AGTAGTCGTATGCTGATGCGCAGTTAGCGTAGCTGAGGTACCGTCGACGATATCGGATCCTTCG
CATATTAAGGTGACGCGTGTGGCCTCGAACACCGAGCGACCCTGCAGCGACCCGCTTAA
AP1 underlined, minimal promoter bold
Synp-FLP-01
(SEQ ID NO: 42)
TGAGTCAGATGATGCGTAGCTAGTAGTTGAGTCAGATGATGCGTAGCTAGTAGTTGAGTCAGATG
ATGCGTAGCTAGTAGTTGAGTCAGATGATGCGTAGCTAGTAGTTGAGTCAGATGATGCGTAGCTA
GTAGTTGAGTCAGATGATGCGTAGCTAGTAGTTGAGTCAGATGATGCGTAGCTAGTAGTTGAGTC
AGTAGTCGTATGCTGATGCGCAGTTAGCGTAGCTGAGGTACCGTCGACGATATCGGATCCGGGC
ATATAAAACAGGGGCAAGGCACAGACTCATAGCAGAGCAATCACCACCAAGCCTGGAATAAC
TGCAGCCACC
AP1 underlined, minimal promoter bold
Synp-RTV-017
(SEQ ID NO: 43)
TGAGTCAGATGATGCGTAGCTAGTAGTTGAGTCAGATGATGCGTAGCTAGTAGTTGAGTCAGATG
ATGCGTAGCTAGTAGTTGAGTCAGATGATGCGTAGCTAGTAGTTGAGTCAGATGATGCGTAGCTA
GTAGTTGAGTCAGATGATGCGTAGCTAGTAGTTGAGTCAGATGATGCGTAGCTAGTAGTTGAGTC
AGTAGTCGTATGCTGATGCGCAGTTAGCGTAGCTGAGGTACCGTCGACGATATCGGATCCAGGT
CTATATAAGCAGAGCTCGTTTAGTGAACCGTCAGATCGCCTAGATACGCCATCCACGCTGTTTT
GACCTCCATAGAAGATCGCCACC
AP1 underlined, minimal promoter bold.
Synp-RTV-019
(SEQ ID NO: 44)
TGACGTGCTGATGATGCGTAGCTAGTAGTTGACGTGCTGATGATGCGTAGCTAGTAGTTGACGT
GCTGATGATGCGTAGCTAGTAGTTGAGTCAGATGATGCGTAGCTAGTAGTTGAGTCAGATGATG
CGTAGCTAGTAGTTGAGTCAGATGATGCGTAGCTAGTAGTTGAGTCAGATGATGCGTAGCTAGTA
GTCTGCACGTAGATGATGCGTAGCTAGTAGTCTGCACGTAGATGATGCGTAGCTAGTAGTCTGC
ACGTAGATGATGCGTAGCTAGTAGTGCAGTTAGCGTAGCTGAGGTACCGTCGACGATATCGGAT
CCAGGTCTATATAAGCAGAGCTCGTTTAGTGAACCGTCAGATCGCCTAGATACGCCATCCACG
CTGTTTTGACCTCCATAGAAGATCGCCACC
Inducible elements underlined, minimal promoter bold.
Synp-FORCYB1
(SEQ ID NO: 45)
CCATTGACGTCACGATTACCATTGACGTCACGATTACCATTGACGTCACGATTACCATTGACGTC
ACGATTACCATTGACGTCAGCGATTAAGATGACTCAGCGATTAAGATGACTCAGCGATTAAGATG
ACTCAGCGATTAAGATGACTCAGCGATTAATCCATATGCTCTAGAGGGTATATAATGGGGGCCA
CTAGTCTACTACCAGAAAGCTTGGTACCGAGCTCGGATCCAGCCACC
cAMPRE underlined, minimal promoter bold.
PM-RQ vector
(SEQ ID NO: 48)
ATGGAAGATGCCAAAAACATTAAGAAGGGCCCAGCGCCATTCTACCCACTCGAAGACGGGACCG
CCGGCGAGCAGCTGCACAAAGCCATGAAGCGCTACGCCCTGGTGCCCGGCACCATCGCCTTTA
CCGACGCACATATCGAGGTGGACATTACCTACGCCGAGTACTTCGAGATGAGCGTTCGGCTGGC
AGAAGCTATGAAGCGCTATGGGCTGAATACAAACCATCGGATCGTGGTGTGCAGCGAGAATAGC
TTGCAGTTCTTCATGCCCGTGTTGGGTGCCCTGTTCATCGGTGTGGCTGTGGCCCCAGCTAACG
ACATCTACAACGAGCGCGAGCTGCTGAACAGCATGGGCATCAGCCAGCCCACCGTCGTATTCGT
GAGCAAGAAAGGGCTGCAAAAGATCCTCAACGTGCAAAAGAAGCTACCGATCATACAAAAGATC
ATCATCATGGATAGCAAGACCGACTACCAGGGCTTCCAAAGCATGTACACCTTCGTGACTTCCCA
TTTGCCACCCGGCTTCAACGAGTACGACTTCGTGCCCGAGAGCTTCGACCGGGACAAAACCATC
GCCCTGATCATGAACAGTAGTGGCAGTACCGGATTGCCCAAGGGCGTAGCCCTACCGCACCGC
ACCGCTTGTGTCCGATTCAGTCATGCCCGCGACCCCATCTTCGGCAACCAGATCATCCCCGACA
CCGCTATCCTCAGCGTGGTGCCATTTCACCACGGCTTCGGCATGTTCACCACGCTGGGCTACTT
GATCTGCGGCTTTCGGGTCGTGCTCATGTACCGCTTCGAGGAGGAGCTATTCTTGCGCAGCTTG
CAAGACTATAAGATTCAATCTGCCCTGCTGGTGCCCACACTATTTAGCTTCTTCGCTAAGAGCAC
TCTCATCGACAAGTACGACCTAAGCAACTTGCACGAGATCGCCAGCGGCGGGGCGCCGCTCAG
CAAGGAGGTAGGTGAGGCCGTGGCCAAACGCTTCCACCTACCAGGCATCCGCCAGGGCTACGG
CCTGACAGAAACAACCAGCGCCATTCTGATCACCCCCGAAGGGGACGACAAGCCTGGCGCAGT
AGGCAAGGTGGTGCCCTTCTTCGAGGCTAAGGTGGTGGACTTGGACACCGGTAAGACACTGGG
TGTGAACCAGCGCGGCGAGCTGTGCGTCCGTGGCCCCATGATCATGAGCGGCTACGTTAACAA
CCCCGAGGCTACAAACGCTCTCATCGACAAGGACGGCTGGCTGCACAGCGGCGACATCGCCTA
CTGGGACGAGGACGAGCACTTCTTCATCGTGGACCGGCTGAAGAGCCTGATCAAATACAAGGG
CTACCAGGTAGCCCCAGCCGAACTGGAGAGCATCCTGCTGCAACACCCCAACATCTTCGACGCC
GGGGTCGCCGGCCTGCCCGACGACGATGCCGGCGAGCTGCCCGCCGCAGTCGTCGTGCTGGA
ACACGGTAAAACCATGACCGAGAAGGAGATCGTGGACTATGTGGCCAGCCAGGTTACAACCGCC
AAGAAGCTGCGCGGTGGTGTTGTGTTCGTGGACGAGGTGCCTAAAGGACTGACCGGCAAGTTG
GACGCCCGCAAGATCCGCGAGATTCTCATTAAGGCCAAGAAGGGCGGCAAGATCGCCGTGTAA
GTGTAATGAAAGCTTGGTCTCTACGAGTAATAGACGCCCAGTTGAATTCCTTCGAGCAGACATGA
TAAGATACATTGATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGTTAACAACAACAA
TTGCATTCATTTTATGTTTCAGGTTCAGGGGGAGGTGTGGGAGGTTTTTTAAAGCAAGTAAAACC
TCTACAAATGTGGTAAAATCGATAAGGATCCGTAACAACAACAATTGCATTCATTTTATGTTTCAG
GTTCAGGGGGAGGTGTGGGAGGTTTTTTAAAGCAAGTAAAACCTCTACAAATGTGGTAAAATCGA
TAAGGATCCGTCTGGGCCTCATGGGCCTTCCGCTCACTGCCCGCTTTCCAGTCGGGAAACCTGT
CGTGCCAGCTGCATTAACATGGTCATAGCTGTTTCCTTGCGTATTGGGCGCTCTCCGCTTCCTCG
CTCACTGACTCGCTGCGCTCGGTCGTTCGGGTAAAGCCTGGGGTGCCTAATGAGCAAAAGGCC
AGCAAAAGGCCAGGAACCGTAAAAAGGCCGCGTTGCTGGCGTTTTTCCATAGGCTCCGCCCCCC
TGACGAGCATCACAAAAATCGACGCTCAAGTCAGAGGTGGCGAAACCCGACAGGACTATAAAGA
TACCAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAGGTAT
CTCAGTTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACTTATCGC
CACTGGCAGCAGCCACTGGTAACAGGATTAGCAGAGCGAGGTATGTAGGCGGTGCTACAGAGT
TCTTGAAGTGGTGGCCTAACTACGGCTACACTAGAAGAACAGTATTTGGTATCTGCGCTCTGCTG
AAGCCAGTTACCTTCGGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGGATCTCAAGAAGATCCT
TTGATCTTTTCTACGGGGTCTGACGCTCAGTGGAACGAAAACTCACGTTAAGGGATTTTGGTCAT
GAGATTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCAGC
GATCTGTCTATTTCGTTCATCCATAGTTGCCTGACTCCCCGTCGTGTAGATAACTACGATACGGG
AGGGCTTACCATCTGGCCCCAGTGCTGCAATGATACCGCGAGAACCACGCTCACCGGCTCCAG
ATTTATCAGCAATAAACCAGCCAGCCGGAAGGGCCGAGCGCAGAAGTGGTCCTGCAACTTTATC
CGCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTC
ATTCAGCTCCGGTTCCCAACGATCAAGGCGAGTTACATGATCCCCCATGTTGTGCAAAAAAGCG
GTTAGCTCCTTCGGTCCTCCGATCGTTGTCAGAAGTAAGTTGGCCGCAGTGTTATCACTCATGGT
TATGGCAGCACTGCATAATTCTCTTACTGTCATGCCATCCGTAAGATGCTTTTCTGTGACTGGTGA
GTACTCAACCAAGTCATTCTGAGAATAGTGTATGCGGCGACCGAGTTGCTCTTGCCCGGCGTCA
ATACGGGATAATACCGCGCCACATAGCAGAACTTTAAAAGTGCTCATCATTGGAAAACGTTCTTC
GGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGCA
AAATGCCGCAAAAAAGGGAATAAGGGCGACACGGAAATGTTGAATACTCATACTCTTCCTTTTTC
AATATTATTGAAGCATTTATCAGGGTTATTGTCTCATGAGCGGATACATATTTGAATGTATTTAGAA
AAATAAACAAATAGGGGTTCCGCGCACATTTCCCCGAAAAGTGCCAC
SEAP coding sequence
(SEQ ID NO: 49)
ATGCTGCTGCTGCTGCTGCTGCTGGGCCTGAGGCTACAGCTCTCCCTGGGCATCATCCCAGTTG
AGGAGGAGAACCCGGACTTCTGGAACCGCGAGGCAGCCGAGGCCCTGGGTGCCGCCAAGAAG
CTGCAGCCTGCACAGACAGCCGCCAAGAACCTCATCATCTTCCTGGGCGATGGGATGGGGGTG
TCTACGGTGACAGCTGCCAGGATCCTAAAAGGGCAGAAGAAGGACAAACTGGGGCCTGAGATA
CCC
pAAV vector:
(SEQ ID NO: 50)
CGATAGATCTAGGAACCCCTAGTGATGGAGTTGGCCACTCCCTCTCTGCGCGCTCGCTCGCTCA
CTGAGGCCGGGCGACCAAAGGTCGCCCGACGCCCGGGCTTTGCCCGGGCGGCCTCAGTGAGC
GAGCGAGCGCGCAGCTGCCTGCAGGCAGCTTGGCACTGGCCGTCGTTTTACAACGTCGTGACT
GGGAAAACCCTGGCGTTACCCAACTTAATCGCCTTGCAGCACATCCCCCTTTCGCCAGCTGGCG
TAATAGCGAAGAGGCCCGCACCGATCGCCCTTCCCAACAGTTGCGCAGCCTGAATGGCGAATG
GCGCCTGATGCGGTATTTTCTCCTTACGCATCTGTGCGGTATTTCACACCGCATAUGTCAAAGCA
ACCATAGTACGCGCCCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTCCCTTCCTTTCTCGCCA
CGTTCGCCGGCTTTCCCCGTCAAGCTCTAAATCGGGGGCTCCCTTTAGGGTTCCGATTTAGTGC
TTTACGGCACCTCGACCCCAAAAAACTTGATTTGGGTGATGGTTCACGTAGTGGGCCATCGCCC
TGATAGACGGTTTTTCGCCCTTTGACGTTGGAGTCCACGTTCTTTAATAGTGGACTCTTGTTCCAA
ACTGGAACAACACTCAACCCTATCTCGGGCTATTCTTTTGATTTATAAGGGATTTTGCCGATTTCG
GCCTATTGGTTAAAAAATGAGCTGATTTAACAAAAATTTAACGCGAATTTTAACAAAATATTAACGT
TTACAATTTTATGGTGCACTCTCAGTACAATCTGCTCTGATGCCGCATAGTTAAGCCAGCCCCGA
CACCCGCCAACACCCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTCATCACCGAAACGCG
CGAGACGAAAGGGCCTCGTGATACGCCTATTTTTATAGGTTAATGTCATGATAATAATGGTTTCTT
AGACGTCAGGTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAATATTGAAAAAGG
AAGAGTATGAGTATTCAACATTTCCGTGTCGCCCTTATTCCCTTTTTTGCGGCATTTTGCCTTCCT
GTTTTTGCTCACCCAGAAACGCTGGTGAAAGTAAAAGATGCTGAAGATCAGTTGGGTGCACGAG
TGGGTTACATCGAACTGGATCTCAACAGCGGTAAGATCCTTGAGAGTTTTCGCCCCGAAGAACG
TTTTCCAATGATGAGCACTTTTAAAGTTCTGCTATGTGGCGCGGTATTATCCCGTATTGACGCCG
GGCAAGAGCAACTCGGTCGCCGCATACACTATTCTCAGAATGACTTGGTTGAGTACTCACCAGT
CACAGAAAAGCATCTTACGGATGGCATGACAGTAAGAGAATTATGCAGTGCTGCCATAACCATGA
GTGATAACACTGCGGCCAACTTACTTCTGACAACGATCGGAGGACCGAAGGAGCTAACCGCTTT
TTTGCACAACATGGGGGATCATGTAACTCGCCTTGATCGTTGGGAACCGGAGCTGAATGAAGCC
ATACCAAACGACGAGCGTGACACCACGATGCCTGTAGCAATGGCAACAACGTTGCGCAAACTAT
TAACTGGCGAACTACTTACTCTAGCTTCCCGGCAACAATTAATAGACTGGATGGAGGCGGATAAA
GTTGCAGGACCACTTCTGCGCTCGGCCCTTCCGGCTGGCTGGTTTATTGCTGATAAATCTGGAG
CCGGTGAGCGTGGGTCTCGCGGTATCATTGCAGCACTGGGGCCAGATGGTAAGCCCTCCCGTA
TCGTAGTTATCTACACGACGGGGAGTCAGGCAACTATGGATGAACGAAATAGACAGATCGCTGA
GATAGGTGCCTCACTGATTAAGCATTGGTAACTGTCAGACCAAGTTTACTCATATATACTTTAGAT
TGATTTAAAACTTCATTTTTAATTTAAAAGGATCTAGGTGAAGATCCTTTTTGATAATCTCATGACC
AAAATCCCTTAACGTGAGTTTTCGTTCCACTGAGCGTCAGACCCCGTAGAAAAGATCAAAGGATC
TTCTTGAGATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCAGCAGAG
CGCAGATACCAAATACTGTTCTTCTAGTGTAGCCGTAGTTAGGCCACCACTTCAAGAACTCTGTA
GCACCGCCTACATACCTCGCTCTGCTAATCCTGTTACCAGTGGCTGCTGCCAGTGGCGATAAGT
CGTGTCTTACCGGGTTGGACTCAAGACGATAGTTACCGGATAAGGCGCAGCGGTCGGGCTGAA
CGGGGGGTTCGTGCACACAGCCCAGCTTGGAGCGAACGACCTACACCGAACTGAGATACCTAC
AGCGTGAGCTATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCCTGGTATCTT
TATAGTCCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTTGCTGGCC
TTTTGCTCACATGTTCTTTCCTGCGTTATCCCCTGATTCTGTGGATAACCGTATTACCGCCTTTGA
GTGAGCTGATACCGCTCGCCGCAGCCGAACGACCGAGCGCAGCGAGTCAGTGAGCGAGGAAG
CGGAAGAGCGCCCAATACGCAAACCGCCTCTCCCCGCGCGTTGGCCGATTCATTAATGCAGCTG
GCACGACAGGTTTCCCGACTGGAAAGCGGGCAGTGAGCGCAACGCAATTAATGTGAGTTAGCTC
ACTCATTAGGCACCCCAGGCTTTACACTTTATGCTTCCGGCTCGTATGTTGTGTGGAATTGTGAG
CGGATAACAATTTCACACAGGAAACAGCTATGACCATGATTACGAATTGCCTGCAGGCAGCTGC
GCGCTCGCTCGCTCACTGAGGCCGCCCGGGCAAAGCCCGGGCGTCGGGCGACCTTTGGTCGC
CCGGCCTCAGTGAGCGAGCGAGCGCGCAGAGAGGGAGTGGCCAACTCCATCACTAGGGGTTC
CTATC

### REFERENCES

Craig, J. C. et al., 2001. Consensus and Variant cAMP-regulated Enhancers Have Distinct CREB-binding Properties. THE JOURNAL OF BIOLOGICAL CHEMISTRY, 276(15), pp. 11719-11728.
Ede, C., Chen, X., Lin, M.-Y. & Chen, Y. Y., 2016. Quantitative Analyses of Core Promoters Enable Precise Engineering of Regulated Gene Expression in Mammalian Cells. ACS Synth Biol., 5(5), p. 395-404.
Hess, H., Angel, P. & Schorpp-Kistner, M., 2004. AP-1 subunits: quarrel and harmony among siblings. Journal of Cell Science, Volume 117, pp. 5965-5973.
Javan, B. & Shanbazi, M., 2017. Hypoxia-inducible tumour-specific promoters as a dual-targeting transcriptional regulation system for cancer gene therapy. Ecancer, 11(751), pp. 1-10.
Kaluz, S., Kaluzová, M. & Stanbridge, E. J., 2008. Rational design of minimal hypoxia-inducible enhancers. Biochem Biophys Res Commun., 370(4), p. 613-618.
Samali, A., FitzGerald, U., Deegan, S. & Gupta, S., 2010. Methods for Monitoring Endoplasmic Reticulum Stress and the Unfolded Protein Response. International Journal of Cell Biology, pp. 1-11.
Schödel, J. et al., 2011. High-resolution genome-wide mapping of HIF-binding sites by ChIP-seq. Blood, 117(23), pp. e207-e217.
Sharma, C. S. & Richards, J. S., 2000. Regulation of AP1 (Jun/Fos) Factor Expression and Activation in. THE JOURNAL OF BIOLOGICAL CHEMISTRY, 275(43), p. 33718-33728.
Yan, K. et al., 2016. The cyclic AMP signaling pathway: Exploring targets for successful drug discovery (Review). MOLECULAR MEDICINE REPORTS, Volume 13, pp. 3715-3723.

1. A synthetic forskolin-inducible promoter comprising one of the following structures:
-TGAGTCA-S₂₀-TGAGTCA-S₂₀-TGAGTCA-S₂₀-TGAGTCA-S₂₀-
TGAGTCA-S₂₀-TGAGTCA-S₂₀-TGAGTCA-S₂₀-TGAGTCA-S₅₉-
CMV-MP;
-TGACGTGCT-S₂₀-TGACGTGCT-S₂₀-TGACGTGCT-S₂₀-
TGAGTCA-S₂₀-TGAGTCA-S₂₀-TGAGTCA-S₂₀-TGAGTCA-S₂₀-
CTGCACGTA-S₂₀-CTGCACGTA-S₂₀-CTGCACGTA-S₆₁-CMV-MP;
or
-TGACGTCA-S₁₀-TGACGTCA-S₁₀-TGACGTCA-S₁₀-
TGACGTCA-S₁₀-TGACGTCA-S₁₀-TGAGTCA-S₁₀-TGAGTCA-S₁₀-
TGAGTCA-S₁₀-TGAGTCA-YB-TATA,
wherein Sₓ represents a spacer sequence of length X nucleotides.

2. A synthetic forskolin-inducible promoter according to claim 1 which comprises one of the following sequences:
(SEQ ID NO: 43)
-TGAGTCAGATGATGCGTAGCTAGTAGTTGAGTCAGATGATGCGTAGCTA
GTAGTTGAGTCAGATGATGCGTAGCTAGTAGTTGAGTCAGATGATGCGTA
GCTAGTAGTTGAGTCAGATGATGCGTAGCTAGTAGTTGAGTCAGATGATG
CGTAGCTAGTAGTTGAGTCAGATGATGCGTAGCTAGTAGTTGAGTCAGTA
GTCGTATGCTGATGCGCAGTTAGCGTAGCTGAGGTACCGTCGACGATATC
GGATCCAGGTCTATATAAGCAGAGCTCGTTTAGTGAACCGTCAGATCGCC
TAGATACGCCATCCACGCTGTTTTGACCTCCATAGAAGATCGCCACC;
(SEQ ID NO: 44)
-TGACGTGCTGATGATGCGTAGCTAGTAGTTGACGTGCTGATGATGCGTA
GCTAGTAGTTGACGTGCTGATGATGCGTAGCTAGTAGTTGAGTCAGATGA
TGCGTAGCTAGTAGTTGAGTCAGATGATGCGTAGCTAGTAGTTGAGTCAG
ATGATGCGTAGCTAGTAGTTGAGTCAGATGATGCGTAGCTAGTAGTCTGC
ACGTAGATGATGCGTAGCTAGTAGTCTGCACGTAGATGATGCGTAGCTAG
TAGTCTGCACGTAGATGATGCGTAGCTAGTAGTGCAGTTAGCGTAGCTGA
GGTACCGTCGACGATATCGGATCCAGGTCTATATAAGCAGAGCTCGTTTA
GTGAACCGTCAGATCGCCTAGATACGCCATCCACGCTGTTTTGACCTCCA
TAGAAGATCGCCACC;
and
(SEQ ID NO: 45)
-TGACGTCACGATTACCATTGACGTCACGATTACCATTGACGTCACGATT
ACCATTGACGTCACGATTACCATTGACGTCAGCGATTAAGATGACTCAGC
GATTAAGATGACTCAGCGATTAAGATGACTCAGCGATTAAGATGACTCAG
CGATTAATCCATATGCTCTAGAGGGTATATAATGGGGGCCACTAGTCTAC
TACCAGAAAGCTTGGTACCGAGCTCGGATCCAGCCACC.

3. An expression cassette comprising a synthetic forskolin-inducible promoter according to any one of claim 1 or 2 operably linked to a transgene.

4. The expression cassette according to claim 3 wherein the transgene encodes a therapeutic protein or polypeptide.

5. A bioprocessing vector comprising a synthetic forskolin-inducible promoter according to any one of claims 1-2 or an expression cassette according to any one of claims 3-4.

6. A cell comprising a synthetic forskolin-inducible promoter according to any one of claims 1-2, an expression cassette according to any one of claims 3-4 or a bioprocessing vector according to claim 5.

7. The cell according to claim 6, wherein the cell is a human cell, optionally a HEK-293 cell, or a murine cell, optionally a NS0 cell or a CHO cell such as a CHO-K1SV cell or CHO-K1SV GS knock out.

8. A population of cells according to any one of claim 6 or 7.

9. A cell culture comprising a population of cells according to claim 8 and medium sufficient to support growth of the cells.

10. A reactor vessel comprising a cell culture according to claim 9.

11. Use of bioprocessing vector according to claim 5, or a cell according to claim 6 or 7 in a bioprocessing method for the manufacture of a product of interest, optionally a therapeutic product.

12. The method for producing an expression product, the method comprising:
a) providing a population of cells comprising an expression cassette comprising a synthetic forskolin-inducible promoter according to any one of claims 1-2 operably linked to a transgene;
b) culturing said population of cells;
c) treating said population of cells so as to induce expression of the transgene present in the expression cassette and thereby produce an expression product; and
d) recovering the expression product from said population of cells.

13. The method of claim 12 wherein step c) comprises administering an inducer to the cells.

14. The method of claim 13 wherein the inducer is an agent that activates adenylyl cyclase.

15. The method of claim 14 wherein the inducer is forskolin or NKH 477.

16. A synthetic hypoxia-inducible promoter comprising one of the following structures:
-CTGCACGTA-S₂₀-CTGCACGTA-S₂₀-CTGCACGTA-S₂₀-
CTGCACGTA-S₂₀-CTGCACGTA-S₂₀-CTGCACGTA-S₅₉-
CMV-MP;
-ACCTTGAGTACGTGCGTCTCTGCACGTATG-S₉-
ACCTTGAGTACGTGCGTCTCTGCACGTATG-S₉-
ACCTTGAGTACGTGCGTCTCTGCACGTATG-S₉-
ACCTTGAGTACGTGCGTCTCTGCACGTATG-S₁₇-YB-TATA;
or
-ACCTTGAGTACGTGCGTCTCTGCACGTATG-S₉-
ACCTTGAGTACGTGCGTCTCTGCACGTATG-S₉-
ACCTTGAGTACGTGCGTCTCTGCACGTATG-S₉-
ACCTTGAGTACGTGCGTCTCTGCACGTATG-S₁₇-CMV-MP,
wherein Sx represents a spacer sequence of length X nucleotides.

17. The synthetic hypoxia-inducible promoter according to claim 16 comprising one of the following sequences:
(SEQ ID NO: 82)
-CTGCACGTAGATGATGCGTAGCTAGTAGTCTGCACGTAGATGATGCGTA
GCTAGTAGTCTGCACGTAGATGATGCGTAGCTAGTAGTCTGCACGTAGAT
GATGCGTAGCTAGTAGTCTGCACGTAGATGATGCGTAGCTAGTAGTCTGC
ACGTAGTAGTCGTATGCTGATGCGCAGTTAGCGTAGCTGAGGTACCGTCG
ACGATATCGGATCCAGGTCTATATAAGCAGAGCTCGTTTAGTGAACCGTC
AGATCGCCTAGATACGCCATCCACGCTGTTTTGACCTCCATAGAAGATCG
CCACC;
(SEQ ID NO: 84)
-ACCTTGAGTACGTGCGTCTCTGCACGTATGGCGATTAAGACCTTGAGTA
CGTGCGTCTCTGCACGTATGGCGATTAAGACCTTGAGTACGTGCGTCTCT
GCACGTATGGCGATTAAGACCTTGAGTACGTGCGTCTCTGCACGTATGGC
GATTAATCCATATGCTCTAGAGGGTATATAATGGGGGCCA;
(SEQ ID NO: 85)
-ACCTTGAGTACGTGCGTCTCTGCACGTATGGCGATTAAGACCTTGAGTA
CGTGCGTCTCTGCACGTATGGCGATTAAGACCTTGAGTACGTGCGTCTCT
GCACGTATGGCGATTAAGACCTTGAGTACGTGCGTCTCTGCACGTATGGC
GATTAATCCATATGCAGGTCTATATAAGCAGAGCTCGTTTAGTGAACCGT
CAGATCGCCTAGATACGCCATCCACGCTGTTTTGACCTCCATAGAAGATC
GCCACC;
(SEQ ID NO: 86)
-GATCTTTGTATTTAATTAAGACCTTGAGTACGTGCGTCTCTGCACGTAT
GGCGATTAAGACCTTGAGTACGTGCGTCTCTGCACGTATGGCGATTAAGA
CCTTGAGTACGTGCGTCTCTGCACGTATGGCGATTAAGACCTTGAGTACG
TGCGTCTCTGCACGTATGGCGATTAATCCATATGCTCTAGAGGGTATATA
ATGGGGGCCACTAGTCTACTACCAGAAAGCTTGGTACCGAGCTCGGATCC
AGCCACC;
or
(SEQ ID NO: 87)
GATCTTTGTATTTAATTAAGACCTTGAGTACGTGCGTCTCTGCACGTATG
GCGATTAAGACCTTGAGTACGTGCGTCTCTGCACGTATGGCGATTAAGAC
CTTGAGTACGTGCGTCTCTGCACGTATGGCGATTAAGACCTTGAGTACGT
GCGTCTCTGCACGTATGGCGATTAATCCATATGCAGGTCTATATAAGCAG
AGCTCGTTTAGTGAACCGTCAGATCGCCTAGATACGCCATCCACGCTGTT
TTGACCTCCATAGAAGATCGCCACC.

18. An expression cassette comprising a synthetic hypoxia-inducible promoter according to any one of claim 16 or 17 operably linked to a transgene.

19. The expression cassette according to claim 18 wherein the transgene encodes a therapeutic protein or polypeptide.

20. A bioprocessing vector comprising a synthetic hypoxia-inducible promoter according to any one of claims 16-17.

21. A cell comprising a synthetic hypoxia-inducible promoter according to any one of claims 16-17, an expression cassette according to claims 18-19 or a bioprocessing vector according to claim 20.

22. The cell according to claim 21, wherein the cell is a human cell, optionally a HEK-293 cell, or a murine cell, optionally NS0 cell or a CHO cell such as a CHO-K1SV cell or CHO-K1SV GS knock out.

23. A population of cells according to claim 21 or 22.

24. A cell culture comprising a population of cells according to claim 23 and medium sufficient to support growth of the cells.

25. A reactor vessel comprising a cell culture according to claim 24.

26. Use of the bioprocessing vector according to claim 20, or a cell according to any one of claim 21 or 22 in a bioprocessing method for the manufacture of a product of interest, optionally a therapeutic product.

27. A method for producing an expression product, the method comprising the steps of:
(a) providing a population of eukaryotic cells, optionally animal cells, optionally mammalian cells, comprising an expression cassette according to claim 18 or 19 or bioprocessing vector according to claim 20;
(b) culturing said population of cells; and
(c) treating said population of cells so as to induce hypoxia in the cells, such that expression from the transgene linked to the hypoxia-inducible promoter is induced and the expression product is produced; and
(d) recovering the expression product.

28. The method of claim 27 wherein step (b) comprises maintaining said population of cells under suitable conditions for proliferation of the cells.

29. The method of any one of claim 27 or 28 comprising introducing into the cell an expression cassette according to claim 18 or 19 or bioprocessing vector according to claim 20.

30. The method of any one of claims 27-29 wherein step (c) comprises treating the cells by reducing the amount of oxygen supplied to the cell, e.g. such that the oxygen tension in the cells is 5% or less.
